SEQUENCE LISTING (53 sequences)

SEQ ID NO: 1 - protein, 672 aa, Homo sapiens
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 2 - protein, 526 aa, Artificial Sequence
  sFGFR3
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKW

SEQ ID NO: 3 - protein, 418 aa, Artificial Sequence
  sFGFR3
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLY

SEQ ID NO: 4 - protein, 301 aa, Artificial Sequence
  Synthetic construct
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKVSLESNASMSSNT

SEQ ID NO: 5 - protein, 349 aa, Artificial Sequence
  Synthetic construct
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPVSLESNASMSSNT

SEQ ID NO: 6 - protein, 301 aa, Artificial Sequence
  Synthetic construct
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHSKVYSDAQPHIQWLKHVEVNGSKVGPDGTPYVTVLKVSLESNASMSSNT

SEQ ID NO: 7 - protein, 512 aa, Artificial Sequence
  Synthetic construct
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKVSLESNASMSSNTSGSGSGSGSGSGSGSVVFPYFPRLGRYNLNFHEAQQACLDQDAVIASFDQLYDAWRGGLDWCNAGWLSDGSVQYPITKPREPCGGQNTVPGVRNYGFWDKDKSRYDVFCFTSNFNGRFYYLIHPTKLTYDEAVQACLNDGAQIAKVGQIFAAWKILGYDRCDAGWLADGSVRYPISRPRRRCSPTEAAVRFVGFPDKKHKLYGVYCFRAYN

SEQ ID NO: 8 - protein, 784 aa, Homo sapiens
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 9 - protein, 784 aa, Homo sapiens
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYRVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 10 - DNA, 2016 bp, Artificial Sequence
  Synthetic construct
gagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacg

SEQ ID NO: 11 - DNA, 1579 bp, Artificial Sequence
  Synthetic construct
gagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtgga

SEQ ID NO: 12 - DNA, 1254 bp, Artificial Sequence
  Synthetic construct
gagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtac

SEQ ID NO: 13 - DNA, 903 bp, Artificial Sequence
  Synthetic construct
gagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaaggtgtccctggagtccaacgcgtccatgagctccaacaca

SEQ ID NO: 14 - DNA, 1131 bp, Artificial Sequence
  Synthetic construct
atgggagtgaaggtgctgttcgccctgatctgtatcgccgtggccgaggccgagtctctgggcacagaacagagagtcgtgggcagagccgccgaagtgcctggacctgaacctggccagcaggaacagctggtgtttggcagcggcgacgccgtggaactgagctgtcctccacctggcggaggccctatgggacctaccgtgtgggtcaaggatggcaccggactggtgcctagcgagagggtgctcgtgggacctcagagactgcaggtgctgaacgccagccacgaggatagcggcgcctacagctgcagacagagactgacacagcgggtgctgtgccacttctccgtcagagtgaccgacgcccctagctccggcgacgatgaggatggcgaagatgaggccgaggacaccggcgtggacacaggcgctccatactggaccagacccgagcggatggacaagaaactgctggccgtgcctgccgccaacaccgtgcggtttagatgtcctgccgccggaaaccccacccccagcatcagctggctgaagaacggcagagagttccggggcgagcacagaatcggcggcatcaagctgagacaccagcagtggtccctcgtgatggaaagcgtggtgcccagcgaccggggcaactacacctgtgtggtggaaaacaagttcggcagcatccggcagacctacaccctggacgtgctggaaagaagcccccacagacccatcctgcaggccggactgcctgccaatcagacagccgtgctgggcagcgacgtggaatttcactgcaaggtgtacagcgacgcccagccccacatccagtggctgaaacacgtggaagtgaacggcagcaaagtgggccccgacggcaccccttatgtgaccgtgctgaaaaccgctggcgccaataccaccgacaaagaactggaagtgctgagcctgcacaacgtgaccttcgaggatgccggcgagtacacctgtctggccggcaacagcatcggcttcagccaccattctgcctggctggtggtgctgcccgtgtccctggaaagcaacgccagcatgagcagcaacaccgactacaaggacgacgacgacaagtgaaagctt

SEQ ID NO: 15 - DNA, 1107 bp, Artificial Sequence
  Synthetic construct
atgatgtcctttgtctctctgctcctggttggcatcctattccatgccacccaggccgagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggctgggctgcctgctaaccagacagcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagtgtccctggagtccaacgcgtccatgagctccaacacatga

SEQ ID NO: 16 - DNA, 987 bp, Artificial Sequence
  Synthetic construct
atgggagtgaaggtgctgttcgccctgatctgtatcgccgtggccgaggccgagtctctgggcacagaacagagagtcgtgggcagagccgccgaagtgcctggacctgaacctggccagcaggaacagctggtgtttggcagcggcgacgccgtggaactgagctgtcctccacctggcggaggccctatgggacctaccgtgtgggtcaaggatggcaccggactggtgcctagcgagagggtgctcgtgggacctcagagactgcaggtgctgaacgccagccacgaggatagcggcgcctacagctgcagacagagactgacacagcgggtgctgtgccacttctccgtcagagtgaccgacgcccctagctccggcgacgatgaggatggcgaagatgaggccgaggacaccggcgtggacacaggcgctccatactggaccagacccgagcggatggacaagaaactgctggccgtgcctgccgccaacaccgtgcggtttagatgtcctgccgccggaaaccccacccccagcatcagctggctgaagaacggcagagagttccggggcgagcacagaatcggcggcatcaagctgagacaccagcagtggtccctcgtgatggaaagcgtggtgcccagcgaccggggcaactacacctgtgtggtggaaaacaagttcggcagcatccggcagacctacaccctggacgtgctggaaagaagcccccacagacccatcctgcaggccggactgcctgccaatcagacagccgtgctgggcagcgacgtggaatttcacagcaaggtgtacagcgacgcccagccccacatccagtggctgaaacacgtggaagtgaacggcagcaaagtgggccccgacggcaccccttatgtgaccgtgctgaaggtgtccctggaaagcaacgccagcatgagcagcaacaccgactacaaggacgacgacgacaagtgaaagctt

SEQ ID NO: 17 - DNA, 1107 bp, Artificial Sequence; identical to SEQ ID NO: 15
  Synthetic construct

SEQ ID NO: 18 - DNA, 1548 bp, Artificial Sequence
  Synthetic construct
gagagcctgggcacagagcagcgggtggtgggaagggccgctgaagtgcctggccctgagcctggccagcaggaacagctggtctttggcagcggcgacgccgtggaactgagctgtcctccacccggcggaggccctatgggccctaccgtgtgggtgaaagacggcaccggcctggtgcccagcgagagggtgctggtgggaccccagcggctgcaggtcctgaacgccagccacgaggacagcggcgcctacagctgcagacagagactgacccagcgggtgctgtgccacttcagcgtgcgcgtgaccgacgcccctagctctggcgacgacgaggacggcgaggacgaggccgaggataccggcgtggacacaggcgccccttactggaccagacccgagcggatggacaagaaactgctggccgtgcctgccgccaacaccgtgcggttcagatgtcccgccgctggcaaccccacccccagcatcagctggctgaagaacggcagagagttccggggcgagcaccggatcggcggcatcaagctgcggcaccagcagtggtccctggtcatggaaagcgtggtgccctccgaccggggcaactacacctgtgtggtggaaaacaagttcggcagcatccggcagacctacaccctggacgtgctggaaagaagcccccaccggcccatcctgcaggctggactgcccgccaatcagacagccgtgctgggcagcgacgtggaatttcactgcaaggtgtacagcgacgcccagccccacatccagtggctcaagcacgtggaagtgaacggcagcaaagtgggccccgacggcaccccttacgtgaccgtgctgaaagtgtccctggaaagcaacgccagcatgagcagcaacacctgaggtaccagcggcagcggctctggctctggaagcggaagcggatctgggagcgtggtgttcccctacttcccccggctgggccggtacaacctgaacttccacgaggcccagcaggcttgcctcgaccaggatgccgtgatcgccagcttcgaccagctgtacgatgcctggcggggaggcctggactggtgcaatgccggctggctgtccgacggcagcgtccagtaccccatcaccaagcccagagagccctgcggcggacagaacacagtgcccggcgtgcggaactacggcttctgggacaaggacaagagcagatacgacgtgttctgcttcaccagcaacttcaacggccggttctactacctgatccaccccaccaagctgacctacgacgaagccgtccaggcctgtctgaacgacggcgcccagatcgccaaagtgggacagatcttcgccgcctggaagatcctgggctacgacagatgcgacgccggatggctggccgatggctccgtgcgctaccctatcagcagaccccgcagaagatgcagccctaccgaggccgccgtgcgcttcgtgggcttccccgacaagaagcacaagctgtacggcgtctactgcttccgggcctacaactga

SEQ ID NO: 19 - DNA, 2355 bp, Homo sapiens
gagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtga

SEQ ID NO: 20 - DNA, 2355 bp, Homo sapiens
  misc_feature (1073)..(1073): N is a or c
gagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacgnggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtga

SEQ ID NO: 21 - protein, 22 aa, Artificial Sequence
  Synthetic construct
MGAPACALALCVAVAIVAGASS

SEQ ID NO: 22 - protein, 672 aa, Artificial Sequence
  Synthetic construct
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHSKVYSDAQPHIQWLKHVEVNGSKVGPDGTPYVTVLKVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 23 - protein, 720 aa, Artificial Sequence
  Synthetic construct
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 24 - protein, 512 aa, Artificial Sequence
  Synthetic construct
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHSKVYSDAQPHIQWLKHVEVNGSKVGPDGTPYVTVLKVSLESNASMSSNTSGSGSGSGSGSGSGSVVFPYFPRLGRYNLNFHEAQQACLDQDAVIASFDQLYDAWRGGLDWCNAGWLSDGSVQYPITKPREPCGGQNTVPGVRNYGFWDKDKSRYDVFCFTSNFNGRFYYLIHPTKLTYDEAVQACLNDGAQIAKVGQIFAAWKILGYDRCDAGWLADGSVRYPISRPRRRCSPTEAAVRFVGFPDKKHKLYGVYCFRAYN

SEQ ID NO: 25 - protein, 560 aa, Artificial Sequence
  Synthetic construct
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPVSLESNASMSSNTSGSGSGSGSGSGSGSVVFPYFPRLGRYNLNFHEAQQACLDQDAVIASFDQLYDAWRGGLDWCNAGWLSDGSVQYPITKPREPCGGQNTVPGVRNYGFWDKDKSRYDVFCFTSNFNGRFYYLIHPTKLTYDEAVQACLNDGAQIAKVGQIFAAWKILGYDRCDAGWLADGSVRYPISRPRRRCSPTEAAVRFVGFPDKKHKLYGVYCFRAYN

SEQ ID NO: 26 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 27 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 28 - protein, 208 aa, Homo sapiens
MAPLGEVGNYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRYYVALNKDGTPREGTRTKRHQKFTHFLPRPVDPDKVPELYKDILSQS

SEQ ID NO: 29 - protein, 207 aa, Homo sapiens
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 30 - protein, 216 aa, Homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 31 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 32 - DNA, 2019 bp, Artificial Sequence
  Synthetic construct
atggagtctctgggcacagagcagagagtcgtgggcagagccgctgaagtgcctggacctgagcctggccagcaggaacagctggtctttggctctggcgacgccgtggaactgagctgtcctccacctggcggaggccctatgggacctaccgtgtgggtcaaggatggcaccggactggtgcctagcgagagggtgctcgtgggacctcagagactgcaggtcctgaacgccagccacgaggatagcggcgcctacagctgcagacagagactgacccagcgggtgctgtgccacttcagcgtcagagtgaccgatgcccccagcagcggagatgacgaggatggcgaggatgaggccgaggatacaggcgtggacacaggcgccccttactggaccagacccgagcggatggacaagaaactgctggccgtgcctgccgccaacaccgtgcggtttagatgccctgccgccggaaaccccacccccagcatctcttggctgaagaacggcagagagttccggggcgagcaccggatcggcggcattaagctgagacaccagcagtggtccctggtcatggaaagcgtggtgcccagcgaccggggcaactacacctgtgtggtggaaaacaagttcggcagcatccggcagacctacaccctggacgtgctggaaagaagcccccacagacctatcctgcaggccggactgcctgccaatcagacagccgtgctgggcagcgacgtggaatttcacagcaaggtgtacagcgacgcccagccccacatccagtggctgaagcacgtggaagtgaacggcagcaaagtgggccccgacggcaccccttacgtgaccgtgctgaaagtgtccctggaaagcaacgccagcatgagcagcaacacccccctcgtgcggatcgccagactgtctagcggagagggccctaccctggccaacgtgtccgaactggaactgcccgccgaccccaagtgggagctgagcagagctagactgaccctgggcaagcctctgggcgagggctgttttggacaggtggtcatggccgaggccatcggcatcgacaaggacagagccgccaagcctgtgaccgtggccgtgaagatgctgaaggacgacgccaccgacaaggacctgagcgacctggtgtccgagatggaaatgatgaagatgatcggcaagcacaagaacatcatcaacctgctgggcgcctgcacccagggcggacctctgtacgtgctggtggaatacgccgccaagggcaacctgagagagttcctgagagccagaaggccccctggcctggactacagcttcgatacctgcaagccccccgaagaacagctgaccttcaaggatctggtgtcctgcgcctatcaggtggccagaggcatggaatacctggccagccagaagtgcatccaccgggatctggccgccagaaacgtgctggtcaccgaggacaacgtgatgaagatcgccgacttcggcctggcccgggacgtgcacaacctggactactacaagaaaaccaccaacggccggctgcccgtgaagtggatggcccctgaggccctgttcgacagagtgtacacccaccagagcgacgtgtggtccttcggcgtgctgctgtgggagatctttaccctgggcggcagcccttaccccggcatccctgtggaagaactgttcaagctgctgaaagagggccacagaatggacaagcccgccaactgcacccacgacctgtacatgatcatgagagagtgctggcacgccgctcccagccagaggcctacctttaagcagctggtggaagatctggaccgggtgctgaccgtgaccagcaccgacgagtacctggatctgagcgcccccttcgagcagtactctcctggcggccaggatacccctagcagcagctctagcggcgacgacagcgtgttcgcccacgatctgctgcctccagcccctcctagctctggcggctctagaacc

SEQ ID NO: 33 - DNA, 1491 bp, Artificial Sequence
  Synthetic construct
atggagagcctgggcacagaacagagagtcgtgggcagagccgccgaagtgcctggacctgaacctggccagcaggaacagctggtctttggctctggcgacgccgtggaactgagctgtcctccacctggcggaggccctatgggacctaccgtgtgggtcaaggatggcaccggactggtgcctagcgagagggtgctcgtgggacctcagagactgcaggtcctgaacgccagccacgaggatagcggcgcctacagctgcagacagagactgacccagcgggtgctgtgccacttcagcgtcagagtgaccgatgcccccagcagcggagatgacgaggatggcgaggatgaggccgaggatacaggcgtggacacaggcgccccttactggaccagacccgagcggatggacaagaaactgctggccgtgcctgccgccaacaccgtgcggtttagatgccctgccgccggaaaccccacccccagcatctcttggctgaagaacggcagagagttccggggcgagcaccggatcggcggcattaagctgagacaccagcagtggtccctggtcatggaaagcgtggtgcccagcgaccggggcaactacacctgtgtggtggaaaacaagttcggcagcatccggcagacctacaccctggacgtgctggaaagaagcccccacagacctatcctgcaggccggactgcctgccaatcagacagccgtgctgggcagcgacgtggaatttcactgcaaggtgtacagcgacgcccagccccacatccagtggctgaagcacgtggaagtgaacggcagcaaagtgggccccgacggcaccccttacgtgaccgtgctgaaagtgtccctggaaagcaacgccagcatgagcagcaacaccagcggcagcggctctggcagcggatctggttctggctccggcagcgtggtgttcccctacttcccccggctgggccggtacaacctgaactttcatgaggcccagcaggcctgcctggaccaggatgccgtgatcgccagcttcgaccagctgtacgatgcttggagaggcggcctggactggtgcaatgccggctggctgtctgacggcagcgtgcagtaccccatcaccaagcccagagagccctgcggcggacagaataccgtgcccggcgtgcggaactacggcttctgggacaaggacaagagcagatacgacgtgttctgcttcaccagcaacttcaacggccggttctactacctgatccaccccaccaagctgacctacgacgaggccgtgcaggcctgtctgaacgatggcgcccagatcgccaaagtgggacagatcttcgccgcctggaagatcctgggctacgacagatgtgacgccggatggctggccgacggctccgtgcggtatcccatcagccggcctagaagaagatgcagccctaccgaggccgccgtcagattcgtgggcttcccc

SEQ ID NO: 34 - DNA, 1710 bp, Artificial Sequence
  Synthetic construct
atgggagcaccagcttgtgccctggctctgtgtgtggccgtggctattgtggctggcgcctctagcgagagcctgggcacagaacagagagtcgtgggcagagccgccgaagtgcctggacctgaacctggccagcaggaacagctggtctttggctctggcgacgccgtggaactgagctgtcctccacctggcggaggccctatgggacctaccgtgtgggtcaaggatggcaccggactggtgcctagcgagagggtgctcgtgggacctcagagactgcaggtcctgaacgccagccacgaggatagcggcgcctacagctgcagacagagactgacccagcgggtgctgtgccacttcagcgtcagagtgaccgatgcccccagcagcggagatgacgaggatggcgaggatgaggccgaggatacaggcgtggacacaggcgccccttactggaccagacccgagcggatggacaagaaactgctggccgtgcctgccgccaacaccgtgcggtttagatgccctgccgccggaaaccccacccccagcatctcttggctgaagaacggcagagagttccggggcgagcaccggatcggcggcattaagctgagacaccagcagtggtccctggtcatggaaagcgtggtgcccagcgaccggggcaactacacctgtgtggtggaaaacaagttcggcagcatccggcagacctacaccctggacgtgctggaaagaagcccccacagacctatcctgcaggccggactgcctgccaatcagacagccgtgctgggcagcgacgtggaatttcactgcaaggtgtacagcgacgcccagccccacatccagtggctgaagcacgtggaagtgaacggcagcaaagtgggccccgacggcaccccttacgtgaccgtgctgaaaaccgctggcgccaacaccaccgacaaagaactggaagtgctgagcctgcacaacgtgaccttcgaggacgccggcgagtacacctgtctggccggcaatagcatcggcttcagccaccactctgcctggctggtggtgctgccaggcggaggctctgtgtccctggaaagcaacgccagcatgagcagcaacaccagcggcagcggctctggcagcggatctggttctggctccggcagcgtggtgttcccctacttcccccggctgggccggtacaacctgaactttcatgaggcccagcaggcctgcctggaccaggatgccgtgatcgccagcttcgaccagctgtacgatgcttggagaggcggcctggactggtgcaatgccggctggctgtctgacggcagcgtgcagtaccccatcaccaagcccagagagccctgcggcggacagaataccgtgcccggcgtgcggaactacggcttctgggacaaggacaagagcagatacgacgtgttctgcttcaccagcaacttcaacggccggttctactacctgatccaccccaccaagctgacctacgacgaggccgtgcaggcctgtctgaacgatggcgcccagatcgccaaagtgggacagatcttcgccgcctggaagatcctgggctacgacagatgtgacgccggatggctggccgacggctccgtgcggtatcccatcagccggcctagaagaagatgcagccctaccgaggccgccgtcagattcgtgggcttcccc

SEQ ID NO: 35 - protein, 236 aa, Homo sapiens
GGGGAGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 236 aa, Artificial Sequence
  Synthetic construct
GGGGAGGGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 585 aa, Homo sapiens [sequence omitted]

SEQ ID NO: 38 - protein, 9 aa, Artificial Sequence
  Synthetic construct
GGGGAGGGG

SEQ ID NO: 39 - protein, 15 aa, Artificial Sequence
  Synthetic construct
GGGGSGGGGSGGGGS

SEQ ID NO: 40 - protein, 208 aa, Homo sapiens
MWKWILTHCASAFPHLPGCCCCCFLLLFLVSSVPVTCQALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 41 - protein, 251 aa, Homo sapiens
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKFI

SEQ ID NO: 42 - protein, 806 aa, Artificial Sequence
  Synthetic construct
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 43 - protein, 19 aa, Artificial Sequence
  Synthetic construct
MMSFVSLLLVGILFHATQA

SEQ ID NO: 44 - DNA, 20 bp, Artificial Sequence
  Synthetic construct
cagatgcactcccatcctcg

SEQ ID NO: 45 - DNA, 20 bp, Artificial Sequence
  Synthetic construct
tctggggatgtccagtaggg

SEQ ID NO: 46 - DNA, 21 bp, Artificial Sequence
  Synthetic construct
tggcagtgaagatgttgaaag

SEQ ID NO: 47 - DNA, 26 bp, Artificial Sequence
  Synthetic construct
atcatcttcatcatctccatctcttg

SEQ ID NO: 48 - DNA, 20 bp, Artificial Sequence
  Synthetic construct
ttatccttggctcctgggtg

SEQ ID NO: 49 - DNA, 20 bp, Artificial Sequence
  Synthetic construct
ctggaaggtagcagtgggaa

SEQ ID NO: 50 - DNA, 21 bp, Artificial Sequence
  Synthetic construct
gctcggaggtagaggtcttgt

SEQ ID NO: 51 - DNA, 19 bp, Artificial Sequence
  Synthetic construct
ccacgctgactggtaggaa

SEQ ID NO: 52 - DNA, 20 bp, Artificial Sequence
  Synthetic construct
tttggtggacacaggcattg

SEQ ID NO: 53 - DNA, 20 bp, Artificial Sequence
  Synthetic construct
caaactgcccgatcatggag

(19) United States
(12) Patent Application Publication
(10) Pub. No.: US 2020/0297799 A1
(43) Pub. Date: Sep. 24, 2020

(54) TREATMENT OF ABNORMAL VISCERAL FAT DEPOSITION USING SOLUBLE FIBROBLAST GROWTH FACTOR RECEPTOR 3 (SFGFR3) POLYPEPTIDES

(71) Applicants: Pfizer Inc., New York, NY (US); Université Côte d'Azur, Nice Cedex 2 (FR); INSERM (Institut National de la Santé et de la Recherche Médicale), Paris (FR); Centre National de la Recherche Scientifique, Paris (FR)

(72) Inventors: Elvire Gouze, Vallauris (FR); Stéphanie Garcia, Nice (FR)

(73) Assignees: Pfizer Inc., New York, NY (US); Université Côte d'Azur, Nice Cedex 2 (FR); INSERM (Institut National de la Santé et de la Recherche Médicale), Paris (FR); Centre National de la Recherche Scientifique, Paris (FR)

(21) Appl. No.: 16/649,208

(22) PCT Filed: Sep. 20, 2018
(86) PCT No.: PCT/EP2018/075471

§ 371 Date: Mar. 20, 2020

Related U.S. Application Data
(60) Provisional application No. 62/561,140, filed on Sep. 20, 2017.

(51) Int. Cl. A61K 38/17; A61K 9/00; A61P 5/00; A61P 19/00

(52) U.S. Cl. CPC A61K 38/179; A61P 19/00; A61P 5/00; A61K 9/0019

(57) ABSTRACT

The invention features methods of using SFGFR3 polypeptides to treat abnormal visceral fat deposition and the conditions associated with abnormal visceral fat deposition.

### BACKGROUND OF THE INVENTION

Achondroplasia, the most common form of short limb dwarfism, is a rare genetic disease for which there is no cure. In many patients, a G380R substitution in the transmembrane domain of the fibroblast growth factor receptor 3 (FGFR3) (Fgfr3ach) results in a gain-of function, prolonging the intracellular MAPK signaling. In the growth plate, the MAPK signaling is inhibitory and its subsequent constitutive activation results in the inhibition of chondrocyte proliferation and differentiation. Cells expressing the mutant receptor do not mature and are not replaced by mineralized bone matrix, ultimately resulting in abnormally short bones.

Achondroplasia is also characterized by early obesity which represents a major health problem in these patients, affecting approximately 50% of patients during childhood. Obesity increases the morbidity associated with lumbar lordosis, as well as the physical impact of existing orthopedic complications, increasing, for example, bearing weight on already fragile knees. It can also increase the risk of serious complications such as cardiovascular risks, obstructive sleep apnea, or restrictive lung disease. The causes of this increased susceptibility to obesity in achondroplasia patients are not known, but does not appear to be linked to hormonal or neurological dysfunction that can lead to appetite deregulation, such as hyperphagia.

Obese achondroplastic patients may also suffer from associated metabolic complications, such as dyslipidemia, low insulin levels, and glucose dysregulation. It is not clear whether these complications are isolated and related to exogenous factors, such as excessive caloric intake and/or decreased physical activity, or if they indeed reflect an underlying defect in achondroplasia.

Abnormal fat deposition, and, more particularly, abnormal visceral fat deposition, in the general population is also associated with the development of specific diseases, including cardiovascular, metabolic, pulmonary, reproductive and neurologic diseases. There exists a need for therapies to treat or prevent the development of abnormal visceral fat deposition and its associated diseases.

### SUMMARY OF THE INVENTION

In a first aspect, the application describes a method of treating or reducing abnormal fat deposition (e.g., visceral fat deposition) in a subject (e.g., a human, such as a fetus, neonate, infant, child, adolescent, or adult) in need thereof by administering a soluble fibroblast growth factor receptor 3 (sFGFR3) polypeptide, a polynucleotide encoding the sFGFR3 polypeptide, or a host cell that contains the polynucleotide encoding the same to the subject. In several embodiments, the abnormal visceral fat deposition is associated with or surrounding one or more of the following organs: the heart, liver, spleen, kidneys, pancreas, intestines, reproductive organs, and gall bladder; or the abnormal visceral fat deposition causes disease in one or more of the following organs: the heart, lungs, trachea, liver, pancreas, brain, reproductive organs, arteries, and gall bladder; or the abnormal visceral fat deposition is caused by dysfunction in an endocrine organ, such as an adrenal gland, a pituitary gland, or a reproductive organ, such as an ovary. The method may result in the reduction or elimination of, or decreased risk of developing, one or more conditions associated with the abnormal fat distribution, such as obstructive sleep apnea, pulmonary disease, cardiovascular disease, metabolic disease, neurological disease, dyslipidemia, hypertension, atherosclerosis, myocardial infarction, stroke, dementia, infertility, menstrual irregularities, insulin dysregulation, and glucose dysregulation. In particular, the dyslipidemia is an abnormal level of one or more of triglycerides, high-density lipoproteins (HDLs), low-density lipoproteins (LDLs), and cholesterol; the cardiovascular disease is heart disease or stroke; the pulmonary disease is asthma and restrictive lung disease; the neurological disease is dementia or Alzheimer's disease; the metabolic disease is type 2 diabetes, glucose intolerance, nonalcoholic fatty liver disease and liver toxicity; the insulin dysregulation is insulin resistance.

In an embodiment, the subject having abnormal fat deposition is not overweight, lacks substantial subcutaneous fat deposition, and/or does may not exhibit substantial abnormal fat deposition outside the abdomen. The abnormal fat deposition may be determined using an anthropometric techniques (e.g., body mass index (BMI) or android:gynoid fat ratio) or imaging (e.g., computed tomography (CT), magnetic resonance imaging (MRI), and dual energy x-ray absorptioometry (DXA), methods which may detect abnormal fat distribution in the absence of other adipose phenotypes.

In other embodiments, the subject may have a skeletal growth retardation disorder, such as an FGFR3-related skeletal disease (e.g., an FGFR3-related skeletal disease that is caused by expression in the patient of a FGFR3 variant that exhibits ligand-dependent overactivation, such as an FGFR3 variant having an amino acid substitution of a glycine residue with an arginine residue at position 358 (G358R), as set forth in SEQ ID NO: 9). The FGFR3-related skeletal disease can include, e.g., achondroplasia, thanatophoric dysplasia type I (TDI), thanatophoric dysplasia type II (TDII), severe achondroplasia with developmental delay and Acanthosis nigricans (SADDEN), hypochondroplasia, a craniosynostosis syndrome (e.g. Muenke syndrome, Crouzon syndrome, and Crouzonodermoskeletal syndrome), and camptodactyly, tall stature, and hearing loss syndrome (CATSHL). The patient with a skeletal growth retardation disorder may have one or more symptoms of the skeletal growth retardation disorder selected from the group consisting of short limbs, short trunk, bowlegs, a waddling gait, skull malformations, cloverleaf skull, craniosynostosis, wormian bones, anomalies of the hands, anomalies of the feet, hitchhiker thumb, and chest anomalies. Excluded from the methods described herein may be a subject with a skeletal growth retardation disorder, such as those described above, e.g., achondroplasia, after the cessation of bone growth in the patient (e.g., a fetus, neonate, infant, child, and/or adolescent subject).

In an alternative embodiment, the patient does not have a skeletal growth retardation disorder, but can be characterized as having obesity, polycystic ovary syndrome, or a form of hypercortisolism, such as Cushing's disease.

In other embodiments, the sFGFR3 has at least 50 consecutive amino acids of an extracellular domain of a naturally occurring fibroblast growth factor receptor 3 (FGFR3) polypeptide (e.g., 100-370 consecutive amino acids of an extracellular domain of the naturally occurring fibroblast growth factor receptor 3 (FGFR3) polypeptide or fewer than 350 amino acids of the extracellular domain of the naturally occurring FGFR3 polypeptide). The sFGFR3 polypeptide may have an Ig-like C2-type domain 1, 2, and/or 3 of the naturally occurring FGFR3 polypeptide. The sFGFR3 polypeptide, in particular, lacks a signal peptide and/or a transmembrane domain, such as the signal peptide and/or transmembrane domain of a naturally occurring FGFR3 polypeptide. Alternatively, the sFGFR3 polypeptide is a mature polypeptide. The naturally occurring FGFR3 polypeptide may have the amino acid sequence of Gen bank Accession No. NP_000133.

The sFGFR3 polypeptide may have 400 consecutive amino acids or fewer of an intracellular domain of a naturally-occurring FGFR3 polypeptide (e.g., between 5 and 399 consecutive amino acids, such as 175, 150, 125, 100, 75, 50, 40, 30, 20, 15, or fewer consecutive amino acids, of the intracellular domain of a naturally-occurring FGFR3 polypeptide). The sFGFR3 polypeptide may have an amino acid sequence with at least 90%, 92%, 95%, 97%, or 99% sequence identity to amino acids 401 to 413 of SEQ ID NO: 8 (e.g., the sFGFR3 polypeptide may have amino acids 401 to 413 of SEQ ID NO: 8). In several embodiments, the sFGFR3 polypeptide lacks a tyrosine kinase domain of a naturally-occurring FGFR3 polypeptide, lacks an intracellular domain of a naturally-occurring FGFR3 polypeptide, or is fewer than 475, 450, 425, 400, 375, 350, 300, 250, 200, 150, or 100 amino acids in length. In other embodiments, the sFGFR3 polypeptide has an amino acid sequence with at least 85% sequence identity (e.g., 86%-100% sequence identity) to amino acids residues 1 to 280 of SEQ ID NO: 8. In yet other embodiments, the sFGFR3 polypeptide has an amino acid sequence with at least 85% sequence identity (e.g., 86%-100% sequence identity) to the sequence of any one of SEQ ID NOs: 1-7. The sFGFR3 polypeptide may also have a signal peptide, such as a signal peptide of a naturally-occurring FGFR3 polypeptide (e.g., a signal peptide having the amino acid sequence of SEQ ID NO: 21). The sFGFR3 polypeptide may also have a heterologous polypeptide (e.g., a fragment crystallizable region of an immunoglobulin (Fc region) or human serum albumin (HSA)).

In still other embodiments, the polynucleotide encoding the sFGFR3 polypeptide may have a nucleic acid sequence with at least 85% and up to 100% sequence identity to the nucleic acid sequence of any one of SEQ ID NOs: 10-18 (e.g., the polynucleotide may consist of the nucleic acid sequence of any one of SEQ ID NOs: 10-18). The polynucleotide may be an isolated polynucleotide and/or may be in a vector (e.g., a vector selected from the group consisting of a plasmid, an artificial chromosome, a viral vector, and a phage vector). The vector may be in a host cell (e.g., an isolated host cell, such as a host cell is from the subject or a HEK 293 cell or CHO cell). The host cell may also be transformed with the sFGFR3-encoding polynucleotide.

In other embodiments, the sFGFR3 polypeptide binds to a fibroblast growth factor (FGF), wherein the FGF is selected from the group consisting of fibroblast growth factor 1 (FGF1), fibroblast growth factor 2 (FGF2), fibroblast growth factor 9 (FGF9), fibroblast growth factor 10 (FGF10), fibroblast growth factor 18 (FGF18), fibroblast growth factor 19 (FGF19), fibroblast growth factor 21 (FGF21), and fibroblast growth factor 23 (FGF23). The binding to the FGF can be characterized by an equilibrium dissociation constant (K_{d}) of about 0.2 nM to about 20 nM, or by a K_{d} of about 1 nM to about 10 nM (e.g., the K_{d} is about 1 nm, about 2 nm, about 3 nm, about 4 nm, about 5 nm, about 6 nm, about 7 nm, about 8 nm, about 9 nm, or about 10 nm).

One aspect of the treatment involves administering an sFGFR3 polypeptide, a polynucleotide encoding an sFGFR3 polypeptide, or a host cell containing a polynucleotide encoding an sFGFR3 polypeptide to a subject, e.g., a human subject, such as a naïve human subject that has not yet been treated with an sFGFR3 polypeptide). The sFGFR3 polypeptide may be administered in a composition containing a pharmaceutically acceptable excipient, carrier, or diluent. Possible doses are from about 0.001 mg/kg to about 30 mg/kg (e.g., about 0.01 mg/kg to about 10 mg/kg). Administration may be daily, weekly, or monthly. Dosing periodicity can be seven times a week, six times a week, five times a week, four times a week, three times a week, twice a week, weekly, every two weeks, or once a month. Route of dosing can be subcutaneous, intravenous, intramuscular, intra-arterial, intrathecal, intraperitoneal, parenteral, enteral, or topical. Repeat administration may be performed.

In other embodiments, the sFGFR3 polypeptide has an in vivo half-life of between about 2 hours to about 25 hours.

A second aspect of the invention features a composition containing a soluble fibroblast growth factor receptor 3 (sFGFR3) polypeptide, a polynucleotide encoding the sFGFR3 polypeptide, or a host cell containing the polynucleotide for treating or reducing abnormal fat distribution in a subject in need thereof (e.g., a human subject), e.g., according to the method of the first aspect of the invention.

A third aspect of the invention features the use of a soluble fibroblast growth factor receptor 3 (sFGFR3) polypeptide, a polynucleotide encoding the sFGFR3 polypeptide, or a host cell containing a polynucleotide encoding the sFGFR3 polypeptide in the manufacture of a medicament for treating or reducing abnormal fat distribution in a subject in need thereof (e.g., a human subject), e.g., according to the method of the first aspect of the invention.

### Definitions

As used herein, “a” and “an” means “at least one” or “one or more” unless otherwise indicated. In addition, the singular forms “a,” “an,” and “the” include plural referents unless the context clearly dictates otherwise.

The phrase “abnormal visceral fat deposition” refers to a level of fat deposition in the omentum, mesentery, retroperitoneum and pericardium, that is greater than a level of fat deposition observed in a normal subject, as determined by anthropometric techniques or imaging techniques (see, e.g., “Methods of Diagnosis, infra., for enumeration of values for each technique defining a cutoff distinguishing normal from abnormal visceral fat deposition). For example, subjects with abnormal visceral fat deposition are those that exhibit a visceral fat deposition value that is equal to or greater than 10% above (for example, 20%, 30%, 40%, 50%, 60%, 70%, 80%, 90%, 100%, 200%, 500%, 750%, 1000% or more above) a cutoff for visceral fat deposition relative to that of a normal subject. Commonly, abnormal visceral fat deposition is associated with a subset of obese patients, e.g., those with a BMI >30 kg/m². Abnormal visceral fat deposition can result from a variety of conditions. These include, e.g., skeletal growth retardation syndromes (e.g., achondroplasia), hypercortisolism (e.g., Cushing's disease), and polycystic ovary syndrome.

As used herein, “about” refers to an amount that is ±10% of the recited value and is preferably ±5% of the recited value, or more preferably ±2% of the recited value. For instance, the term “about” can be used to modify all dosages or ranges recited herein by ±10% of the recited values or range endpoints.

The phrase “anthropometric techniques” refers to body composition measurements based on height, weight, waist circumference and hip circumference, including body mass index (BMI), android:gynoid fat ratio, waist circumference, and sagittal diameter (SD); see, e.g., Shuster et al., Br. J. Radiol. 85(1009):1-10, 2012).

The phrase “cardiovascular disease” refers to diseases of the heart and blood vessels, in particular, atherosclerosis, myocardial infarction, and hypertension.

The phrase “conditions associated with abnormal visceral fat deposition” refers to diseases observed in patients whose visceral fat deposition is shown to be abnormal relative to normal patients. In particular, these conditions include cardiovascular disease, pulmonary disease, metabolic disease, reproductive disease, and neurologic disease.

The term “domain” refers to a conserved region of the amino acid sequence of a polypeptide (e.g. a FGFR3 polypeptide) having an identifiable structure and/or function within the polypeptide. A domain can vary in length from, e.g., about 20 amino acids to about 600 amino acids. Exemplary domains include the immunoglobulin domains of a FGFR3 (e.g., Ig-like C2-type domain 1, Ig-like C2-type domain 2, and Ig-like C2-type domain 3), the extracellular domain (ECD) of a FGFR3, the intracellular domain (ICD) of a FGFR3, or the transmembrane domain™ of a FGFR3, such as a FGFR3 having the sequence set forth in SEQ ID NO: 8).

The term “dosage” refers to a determined quantity of an active agent (e.g., an sFGFR3 polypeptide or variant thereof, such as a polypeptide having the amino acid sequence of any one of SEQ ID NOs: 1-7 or a variant thereof having at least 85% to 100% sequence identity thereto) calculated to produce a desired therapeutic effect (e.g., treatment of abnormal visceral fat deposition, or the conditions associated with visceral fat deposition) when the active agent is administered to a patient (e.g., a patient having abnormal visceral fat deposition, or a condition associated with visceral fat deposition). A dosage may be defined in terms of a defined amount of the active agent or a defined amount coupled with a particular frequency of administration. A dosage form can include an sFGFR3 polypeptide or fragment thereof in association with any suitable pharmaceutical excipient, carrier, or diluent.

The terms “effective amount,” “amount effective to,” and “therapeutically effective amount” refer to an amount of an sFGFR3 polypeptide, a vector encoding an sFGR3, and/or an sFGFR3 composition that is sufficient to produce a desired result, for example, decreased abnormal fat deposition, abnormal visceral fat deposition, or a decrease in symptoms associated with conditions linked to abnormal visceral fat deposition.

The terms “extracellular domain” and “ECD” refer to the portion of a FGFR3 polypeptide that extends beyond the transmembrane domain into the extracellular space. The ECD mediates binding of a FGFR3 to one or more fibroblast growth factors (FGFs). For instance, an ECD includes the Ig-like 02-type domains 1-3 of a FGFR3 polypeptide. In particular, the ECD includes the Ig-like C2-type domain 1 of a wildtype (wt) FGFR3 polypeptide, the Ig-like C2-type domain 2 of a wildtype (wt) FGFR3 polypeptide, and/or the Ig-like C2-type domain 3 of a wt FGFR3 polypeptide. An ECD of a FGFR3 can also include a fragment of the wildtype FGFR3 Ig-like 02-type domain for instance.

The phrase “fat deposition” refers to visceral fat deposition or subcutaneous fat deposition.

The term “FGFR3-related skeletal disease,” as used herein, refers to a skeletal disease that is caused by an abnormal increase in the activation of FGFR3, such as by expression of a gain-of-function mutant of the FGFR3. The phrase “gain-of-function mutant of the FGFR3” refers to a mutant of the FGFR3 exhibiting a biological activity, such as triggering downstream signaling, which is higher than the biological activity of the corresponding wild-type FGFR3 (e.g., a polypeptide having the amino acid sequence of SEQ ID NO: 8) in the presence of a FGF ligand. FGFR3-related skeletal diseases can include an inherited or a sporadic disease. Exemplary FGFR3-related skeletal diseases include, but are not limited to, achondroplasia, thanatophoric dysplasia type I (TDI), thanatophoric dysplasia type II (TDII), severe achondroplasia with developmental delay and Acanthosis nigricans (SADDAN), hypochondroplasia, a craniosynostosis syndrome (e.g., Muenke syndrome, Crouzon syndrome, and Crouzonodermoskeletal syndrome), and camptodactyly, tall stature, and hearing loss syndrome (CATSHL).

The terms “fibroblast growth factor” and “FGF” refer to a member of the FGF family, which includes structurally related signaling molecules involved in various metabolic processes, including endocrine signaling pathways, development, wound healing, and angiogenesis. FGFs play key roles in the proliferation and differentiation of a wide range of cell and tissue types. The term preferably refers to FGF1, FGF2, FGF9, FGF 10, FGF18, FGF19, FGF21, and FGF23, which have been shown to bind FGFR3. For instance, FGFs can include human FGF1 (e.g., a polypeptide having the amino acid sequence of SEQ ID NO: 26), human FGF2 (e.g., a polypeptide having the amino acid sequence of SEQ ID NO: 27), human FGF9 (e.g., a polypeptide having the amino acid sequence of SEQ ID NO: 28), human FGF10 (e.g., a polypeptide having the amino acid sequence of SEQ ID NO: 40), human FGF18 (e.g., a polypeptide having the amino acid sequence of SEQ ID NO: 29), human FGF19 (e.g., a polypeptide having the amino acid sequence of SEQ ID NO: 30), human FGF21 (e.g., a polypeptide having the amino acid sequence of SEQ ID NO: 31), and human FGF23 (e.g., a polypeptide having the amino acid sequence of SEQ ID NO: 41).

The terms “fibroblast growth factor receptor 3,” “FGFR3,” or “FGFR3 receptor,” as used herein, refer to a polypeptide that specifically binds one or more FGFs (e.g., FGF1, FGF2, FGF9, FGF10, FGF18, FGF19, FGF 21, and/or FGF23). The human FGFR3 gene, which is located on the distal short arm of chromosome 4, encodes an 806 amino acid protein precursor (fibroblast growth factor receptor 3 isoform 1 precursor), which contains 19 exons, and includes a signal peptide (e.g., a polypeptide having the amino acid sequence of SEQ ID NO: 21). Mutations in the FGFR3 amino acid sequence that lead to skeletal growth disorders, (e.g., achondroplasia), include, e.g., the substitution of a glycine residue at position 358 with an arginine residue (i.e., G358R; SEQ ID NO: 9). The naturally occurring human FGFR3 gene has a nucleotide sequence as shown in Genbank Accession number NM_000142.4 and the naturally occurring human FGFR3 protein has an amino acid sequence as shown in Genbank Accession number NP_000133, herein represented by SEQ ID NO: 8. The wildtype FGFR3 (e.g., a polypeptide having the amino acid sequence of SEQ ID NO: 8) consists of an extracellular immunoglobulin-like membrane domain including Ig-like C2-type domains 1-3, a transmembrane domain, and an intracellular domain. FGFR3s can include fragments and/or variants (e.g., splice variants, such as splice variants utilizing alternate exon 8 rather than exon 9) of the full-length, wild-type FGFR3.

The terms “fragment” and “portion” refer to a part of a whole, such as a polypeptide or nucleic acid molecule that contains, preferably, at least 10%, 20%, 30%, 40%, 50%, 60%, 70%, 75%, 80%, 85%, 90%, 95%, 96%, 97%, 98%, 99%, or more of the entire length of the reference nucleic acid molecule or polypeptide, or a domain thereof (e.g., the ECD, ICD, or TM of a sFGFR3 polypeptide). A fragment or portion may contain, e.g., 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 500, 600, 700, or more consecutive amino acid residues, up to the entire length of the reference polypeptide. For example, a FGFR3 fragment can include any polypeptide having at least about 5 consecutive amino acids to about 300 consecutive amino acids, inclusive of the endpoints, e.g., at least about 10, 20, 30, 40, 50, 60, 70, 80, 90, 100, 110, 120, 130, 140, 150, 160, 170, 180, 190, 200, 205, 210, 215, 220, 225, 230, 235, 240, 245, 250, 255, 260, 265, 270, 275, 280, 285, 290, 295, or 300 consecutive amino acids of any one of SEQ ID Nos: 1-8.

The phrase “glucose dysregulation” refers to a glucose level in the blood that is above or below an acceptable normal range.

As used herein, the term “host cell” refers to a vehicle that includes the necessary cellular components, e.g., organelles, needed to express an sFGFR3 polypeptide from a corresponding polynucleotide. The nucleic acid sequence of the polynucleotide is typically included in a nucleic acid vector (e.g., a plasmid, an artificial chromosome, a viral vector, or a phage vector) that can be introduced into the host cell by conventional techniques known in the art (e.g., transformation, transfection, electroporation, calcium phosphate precipitation, and direct microinjection). A host cell may be a prokaryotic cell, e.g., a bacterial or an archaeal cell, or a eukaryotic cell, e.g., a mammalian cell (e.g., a Chinese Hamster Ovary (CHO) cell or a Human Embryonic Kidney 293 (HEK 293)). Preferably, the host cell is a mammalian cell, such as a CHO cell.

The phrase “imaging techniques” refers to methods of creating visual representations of the interior of a body for the purpose of clinical analysis and medical intervention. Examples of imaging techniques include, e.g., dual energy x-ray absorptiometry (DXA) yielding fat mass index, cross-sectional imaging, such as computed tomography (CT) and magnetic resonance imaging (MRI), yielding an area of visceral fat in cm² at a specified level of the lumbar spine (see, e.g., Shuster et al., supra).

The phrase “insulin dysregulation” refers to an insulin level in the blood that is above or below an acceptable normal range.

By “isolated” is meant separated, recovered, or purified from its natural environment. For example, an isolated sFGFR3 polypeptide (e.g., an sFGFR3 polypeptide or variant thereof, such as a polypeptide having the amino acid sequence of any one of SEQ ID Nos: 1-7 or a variant thereof having at least about 85% to up to about 100% sequence identity thereto) can be characterized by a certain degree of purity after isolating the sFGFR3 polypeptide from, e.g., cell culture media. An isolated sFGFR3 polypeptide can be at least 75% pure, such that the sFGFR3 polynucleotide constitutes at least 75% by weight of the total material (e.g., polypeptides, polynucleotides, cellular debris, and environmental contaminants) present in the preparation (e.g., at least 80%, at least 85%, at least 90%, at least 95%, at least 96%, at least 97%, at least 99%, or at least 99.5% by weight of the total material present in the preparation). Likewise, an isolated polynucleotide encoding an sFGFR3 polypeptide (e.g., a polynucleotide having the nucleic acid sequence of any one of SEQ ID NOs: 10-18 or a variant thereof having at least about 85% to up to about 100% sequence identity thereto), or an isolated host cell (e.g., CHO cell, a HEK 293 cell, L cell, C127 cell, 3T3 cell, BHK cell, COS-7 cell, or a cell of a subject) containing the polynucleotide can be at least 75% pure, such that the polynucleotide or host cell constitutes at least 75% by weight of the total material (e.g., polypeptides, polynucleotides, cellular debris, and environmental contaminants) present in the preparation (e.g., at least 80%, at least 85%, at least 90%, at least 95%, at least 96%, at least 97%, at least 99%, or at least 99.5% by weight of the total material present in the preparation).

The phrase “metabolic disease” refers to disorders of chemical reactions that help with energy processing, in particular dyslipidemia, insulin dysregulation, glucose dysregulation, non-alcoholic fatty liver, and liver toxicity.

The phrase “neurologic disease” refers to diseases of the brain or nerves, in particular, dementia and stroke.

The terms “parenteral administration,” “administered parenterally,” and other grammatically equivalent phrases, as used herein, refer to a mode of administration of compositions including an sFGFR3 polypeptide (e.g., an sFGFR3 polypeptide or variant thereof, such as a polypeptide having the amino acid sequence of any one of SEQ ID NOs: 1-7 (with or without a signal peptide) other than enteral and topical administration, usually by injection, and include, without limitation, subcutaneous, intradermal, intravenous, intranasal, intraocular, pulmonary, intramuscular, intra-arterial, intrathecal, intracapsular, intraorbital, intracardiac, intradermal, intrapulmonary, intraperitoneal, transtracheal, subcuticular, intraarticular, subcapsular, subarachnoid, intraspinal, epidural, intracerebral, intracranial, intracarotid, and intrasternal injection and infusion.

The terms “patient” and “subject” refer to a mammal, including, but not limited to, a human (e.g., a human having abnormal fat deposition, abnormal visceral fat deposition, or the conditions associated with abnormal visceral fat deposition) or a non-human mammal (e.g., a non-human mammal having abnormal fat deposition, abnormal visceral fat deposition, or the conditions associated with abnormal visceral fat deposition, such as a bovine, equine, canine, ovine, or feline. Preferably, the patient is a human having abnormal fat deposition, abnormal visceral fat deposition, or the conditions associated with abnormal visceral fat deposition), particularly a fetus, a neonate, an infant, a child, an adolescent, or an adult having abnormal fat deposition, abnormal visceral fat deposition, or the conditions associated with abnormal visceral fat deposition.

By “pharmaceutical composition” is meant a composition containing an active agent, such as an sFGFR3, formulated with at least one pharmaceutically acceptable excipient, carrier, or diluent. The pharmaceutical composition may be manufactured or sold with the approval of a governmental regulatory agency as part of a therapeutic regimen for the treatment of a disease or event (e.g., abnormal fat deposition, abnormal visceral fat deposition, or the conditions associated with abnormal visceral fat deposition) in a patient (e.g., a patient having abnormal fat deposition, abnormal visceral fat deposition, or the conditions associated with abnormal visceral fat deposition). Pharmaceutical compositions can be formulated, e.g., for parenteral administration, such as for subcutaneous administration (e.g. by subcutaneous injection) or intravenous administration (e.g., as a sterile solution free of particulate emboli and in a solvent system suitable for intravenous use), or for oral administration (e.g., as a tablet, capsule, caplet, gelcap, or syrup).

By “pharmaceutically acceptable diluent, excipient, carrier, or adjuvant” is meant a diluent, excipient, carrier, or adjuvant, respectively that is physiologically acceptable to the subject (e.g., a human) while retaining the therapeutic properties of the pharmaceutical composition (e.g., an sFGFR3 polypeptide or variant thereof, with which it is administered. One exemplary pharmaceutically acceptable carrier is physiological saline. Other physiologically acceptable diluents, excipients, carriers, or adjuvants and their formulations are known to one skilled in the art.

“Polynucleotide” and “nucleic acid molecule,” as used interchangeably herein, refers to polymers of nucleotides of any length, and include DNA and RNA. The nucleotides can be deoxyribonucleotides, ribonucleotides, modified nucleotides or bases, and/or analogs thereof, or any substrate that can be incorporated into a polymer by DNA or RNA polymerase or by a synthetic reaction. A polynucleotide can include modified nucleotides, such as methylated nucleotides and analogs thereof. If present, modification to the nucleotide structure can be imparted before or after assembly of the polymer. The sequence of nucleotides can be interrupted by non-nucleotide components. A polynucleotide can be further modified after synthesis, such as by conjugation with a label.

The phrase “pulmonary disease” refers to diseases of air exchange, in particular, obstructive sleep apnea, restrictive lung disease, and asthma.

The phrase “reproductive disease” refers to diseases of the reproductive system, in particular, infertility and menstrual irregularities.

As used herein, the term “sequence identity” refers to the percentage of amino acid (or nucleic acid) residues of a candidate sequence, e.g., an FGFR3 polypeptide, that are identical to the amino acid (or nucleic acid) residues of a reference sequence, e.g., a wild-type sFGFR3 polypeptide (e.g., a polypeptide having the amino acid sequence of SEQ ID NO: 8) or an sFGFR3 polypeptide (e.g., an sFGFR3 polypeptide or variant thereof, such as a polypeptide having the amino acid sequence of any one of SEQ ID NOs: 1-7) after aligning the sequences and introducing gaps, if necessary, to achieve the maximum percent identity (e.g., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software, such as BLAST, BLAST-2, BLAST-P, BLAST-N, BLAST-X, WU-BLAST-2, ALIGN, ALIGN-2, CLUSTAL, or Megalign (DNASTAR) software. Those skilled in the art can determine appropriate parameters for measuring alignment, including any algorithms needed to achieve maximal alignment over the full length of the sequences being compared. For instance, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows: 100×(fraction of A/B) where A is the number of amino acid (or nucleic acid) residues scored as identical in the alignment of the candidate sequence and the reference sequence, and where B is the totalnumber of amino acid (or nucleic acid) residues in the reference sequence. In particular, areference sequence aligned for comparison with a candidate sequence can show that the candidate sequence exhibits from, e.g., 50% to 100% identity across the full length of the candidate sequence or a selected portion of contiguous amino acid (or nucleic acid) residues of the candidate sequence. The length of the candidate sequence aligned for comparison purposeis at least 30%, e.g., at least 40%, e.g., at least 50%, 60%, 70%, 80%, 90%, or 100% of the length of the reference sequence. When a position in the candidate sequence is occupied by the same amino acid (or nucleic acid) residue as the corresponding position in the reference sequence, then the molecules are identical at that position.

By “signal peptide” is meant a short peptide (e.g., 5-30 amino acids in length, such as 22 amino acids in length) at the N-terminus of a polypeptide that directs a polypeptide towards the secretory pathway (e.g., the extracellular space). The signal peptide is typically cleaved during secretion of the polypeptide. The signal sequence may direct the polypeptide to an intracellular compartment or organelle, e.g., the Golgi apparatus. A signal sequence may be identified by homology, or biological activity, to a peptide with the known function of targeting a polypeptide to a particular region of the cell. One of ordinary skill in the art can identify a signal peptide by using readily available software (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). A signal peptide can be one that is, for example, substantially identical to the amino acid sequence of SEQ ID NO: 21.

The term “skeletal growth retardation disorder,” as used herein, refers to a skeletal disease characterized by deformities and/or malformations of the bones. These disorders include, but are not limiting to, skeletal growth retardation disorders caused by growth plate (physeal) fractures, idiopathic skeletal growth retardation disorders, or FGFR3-related skeletal diseases. In particular, a patient having a skeletal growth retardation disorder (e.g., achondroplasia) may have bones that are shorter than the bones of a healthy patient. For example, the skeletal growth retardation disorder may include a skeletal dysplasia, e.g., achondroplasia, homozygous achondroplasia, heterozygous achondroplasia, achondrogenesis, acrodysostosis, acromesomelic dysplasia, atelosteogenesis, camptomelic dysplasia, chondrodysplasia punctata, rhizomelic type of chondrodysplasia punctata, cleidocranial dysostosis, congenital short femur, craniosynostosis (e.g., Muenke syndrome, Crouzon syndrome, Apert syndrome, Jackson-Weiss syndrome, Pfeiffer syndrome, or Crouzonodermoskeletal syndrome), dactyly, brachydactyly, camptodactyly, polydactyly, syndactyly, diastrophic dysplasia, dwarfism, dyssegmental dysplasia, enchondromatosis, fibrochondrogenesis, fibrous dysplasia, hereditary multiple exostoses, hypochondroplasia, hypophosphatasia, hypophosphatemic rickets, Jaffe-Lichtenstein syndrome, Kniest dysplasia, Kniest syndrome, Langer-type mesomelic dysplasia, Marfan syndrome, McCune-Albright syndrome, micromelia, metaphyseal dysplasia, Jansen-type metaphyseal dysplasia, metatrophic dysplasia, Morquio syndrome, Nievergelt-type mesomelic dysplasia, neurofibromatosis, osteoarthritis, osteochondrodysplasia, osteogenesis imperfecta, perinatal lethal type of osteogenesis imperfecta, osteopetrosis, osteopoikilosis, peripheral dysostosis, Reinhardt syndrome, Roberts syndrome, Robinow syndrome, short-rib polydactyly syndromes, short stature, spondyloepiphyseal dysplasia congenita, and spondyloepimetaphyseal dysplasia.

The terms “soluble fibroblast growth factor receptor 3,” “soluble FGFR3,” and “sFGFR3” refer to a FGFR3 that is characterized by the absence or functional disruption of all or a substantial part of the transmembrane domain and any polypeptide portion that would anchor the FGFR3 polypeptide to a cell membrane (e.g., a tyrosine kinase domain). An sFGFR3 polypeptide is a non-membrane bound form of an FGFR3 polypeptide. Thus, an sFGFR3 polypeptide can include a deletion of a portion or all of the amino acid residues of the transmembrane domain of a wild-type FGFR3 polypeptide sequence (e.g., a polypeptide having the amino acid sequence of SEQ ID NO: 8). The sFGFR3 polypeptide can further include deletions of the intracellular domain of the wild-type FGFR3 polypeptide.

Exemplary sFGFR3 polypeptides can include, but are not limited to, at least amino acids 1 to 100, 1 to 125, 1 to 150, 1 to 175, 1 to 200, 1 to 205, 1 to 210, 1 to 215, 1 to 220, 1 to 225, 1 to 230, 1 to 235, 1 to 240, 1 to 245, 1 to 250, 1 to 252, 1 to 255, 1 to 260, 1 to 265, 1 to 270, 1 to 275, 1 to 280, 1 to 285, 1 to 290, 1 to 295, or 1 to 300, or 1 to 301 of SEQ ID NOs: 1-8. sFGFR3 polypeptides can include any polypeptide having at least 50% (e.g., 55%, 60%, 65%, 70%, 75%, 80%, 85%, 86%, 87%, 88%, 89%, 90%, 91%, 92%, 93%, 94%, 95%, 96%, 97%, 98%, 99%, or more) sequence identity to any of these sFGFR3 polypeptides of SEQ ID NOs: 1-8. Additionally, exemplary sFGFR3 polypeptides can include, but are not limited to, at least amino acids 1 to 100, 1 to 125, 1 to 150, 1 to 175, 1 to 200, 1 to 205, 1 to 210, 1 to 215, 1 to 220, 1 to 225, 1 to 230, 1 to 235, 1 to 240, 1 to 245, 1 to 250, 1 to 255, 1 to 260, 1 to 265, 1 to 270, 1 to 275, 1 to 280, 1 to 285, 1 to 290, 1 to 295, 1 to 300, 1 to 305, 1 to 310, 1 to 315, 1 to 320, 1 to 325, 1 to 330, 1 to 335, 1 to 340, 1 to 345, or 1 to 348 of SEQ ID NOs: 1-8. sFGFR3 polypeptides can include any polypeptide having at least 50% (e.g., 55%, 60%, 65%, 70%, 75%, 80%, 85%, 86%, 87%, 88%, 89%, 90%, 91%, 92%, 93%, 94%, 95%, 96%, 97%, 98%, 99%, or more) sequence identity to any of these sFGFR3 polypeptides having the amino acid sequence of SEQ ID NOs: 1-8. Any of the above sFGFR3 polypeptides or variants thereof can optionally include a signal peptide at the N-terminal position, such as amino acids 1 to 22 of SEQ ID NO: 21 (MGAPACALALCVAVAIVAGASS) or amino acids 1 to 19 of SEQ ID NO: 43 (e.g., MMSFVSLLLVGILFHATQA).

The phrase “subcutaneous fat deposition” refers to fat deposition in the hypodermis.

By “treating” and “treatment” is meant a reduction (e.g., by at least about 10%, about 15%, about 20%, about 25%, about 30%, about 35%, about 40%, about 45%, about 50%, about 60%, about 70%, about 80%, about 90%, about 95%, about 99%, or even 100%) in abnormal fat deposition or abnormal visceral fat deposition, or in the progression, severity, or frequency of one or more or a condition associated with abnormal visceral fat deposition (e.g. cardiovascular disease, pulmonary disease, metabolic disease, or neurological disease) in a patient (e.g., a human, such as a fetus, a neonate, an infant, a child, an adolescent, or an adult). Treatment can occur for a treatment period, in which an sFGFR3 polypeptide is administered for a period of time (e.g., days, months, years, or longer) to treat a patient (e.g., a human, such as a fetus, a neonate, an infant, a child, an adolescent, or an adult) having abnormal fat deposition, abnormal visceral fat deposition, or a condition associated with abnormal visceral fat deposition. Exemplary symptoms of associated with abnormal visceral fat deposition in an achondroplasia patient that can be treated with an sFGFR3 (e.g., an sFGFR3 polypeptide or variant thereof, such as a polypeptide having the amino acid sequence of SEQ ID NOs: 1-7 or a variant thereof) include, but are not limited to, atherosclerosis, hypertension, lipid dysregulation, obstructive sleep apnea, glucose dysregulation or insulin dysregulation (e.g., insulin resistance).

The term “unit dosage form(s)” refers to physically discrete unit(s) suitable as unitary dosages for human subjects and other mammals, each unit containing a predetermined quantity of active material calculated to produce the desired therapeutic effect, in association with any suitable pharmaceutical excipient, carrier, or diluent.

The term “variant,” with respect to a polypeptide, refers to a polypeptide (e.g., an sFGFR3 polypeptide or variant thereof, with or without a signal peptide) that differs by one or more changes in the amino acid sequence from the polypeptide from which the variant is derived (e.g., the reference polypeptide, such as, e.g., a polypeptide having the amino acid sequence of any one of SEQ ID NOs: 1-7). The term “variant,” with respect to a polynucleotide, refers to a polynucleotide that differs by one or more changes in the nucleic acid sequence from the polynucleotide from which the variant is derived (e.g., the reference polynucleotide, such as, e.g., a polynucleotide encoding a sFGFR3 polypeptide having the nucleic acid sequence of any one of SEQ ID NOs: 10-18). The changes in the amino acid or nucleic acid sequence of the variant can be, e.g., amino acid or nucleic acid substitutions, insertions, deletions, N-term inal truncations, or C-terminal truncations, or any combination thereof. In particular, the amino acid substitutions may be conservative and/or non-conservative substitutions. A variant can be characterized by amino acid sequence identity or nucleic acid sequence identity to the reference polypeptide or parent polynucleotide, respectively. For example, a variant can include any polypeptide having at least 50% (e.g., 55%, 60%, 65%, 70%, 75%, 80%, 85%, 86%, 87%, 88%, 89%, 90%, 91%, 92%, 93%, 94%, 95%, 96%, 97%, 98%, 99%, or more) sequence identity to the reference polypeptide or polynucleotide.

By “vector” is meant a DNA construct that includes one or more polynucleotides, or fragments thereof, encoding an sFGFR3 polypeptide (e.g., an sFGFR3 polypeptide or variant thereof, such as a polypeptide having the amino acid sequence of any one of SEQ ID NOs: 1-7, or a variant thereof, with our without a signal peptide). The vector can be used to infect a cell (e.g., a host cell or a cell of a patient having a human skeletal growth retardation disorder, such as achondroplasia), which results in the translation of the polynucleotides of the vector into a sFGFR3 polypeptide. One type of vector is a “plasmid,” which refers to a circular double stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. In general, expression vectors of utility in recombinant DNA techniques are often in the form of plasm ids.

The phrase “visceral fat deposition” refers to intraabdominal fat depots, including mesenteric and omental, retroperitoneal fat depots, and intrathoracic fat depots, including pericardial.

The recitation herein of numerical ranges by endpoints is intended to include all numbers subsumed within that range (e.g., a recitation of 1 to 5 includes 1, 1.5, 2, 2.75, 3, 3.80, 4, and 5).

Other features and advantages of the invention will be apparent from the following Detailed Description and from the Claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D are tables, an image, and graphs showing that children with achondroplasia develop abdominal obesity without an increase in blood glucose levels. FIG. 1A is a table showing weight, height and BMI measurements and corresponding height-to-age and BMI-to-age z-scores in the three age groups ranging (n=73 data points in the [0-3] years old group, n=61 data points in the [4-8] years old group and n=36 data points in the [9-18] years old group). Results of post hoc analyses: a: significantly different between [0-3] and [4-8] groups; b: significantly different between [0-3] and [9-18] groups; c: significantly different between [4-8] and [9-18] groups. FIG. 1B is an image representation of the different regions of interest (ROI) evaluated by DXA. FIG. 1C is a graph showing android:gynoid fat ratio measurement in the three age groups ranging (n=4 data points in the [0-3] years old group, n=6 data points in the [4-8] years old group and n=9 data points in the [9-18] years old group). FIG. 1D is a graph showing plasmatic fasting glucose concentration in the two age groups ranging [4-8] and [9-18] years old (n=16 data points in the [4-8] years old group and n=12 data points in the [9-18] years old group). Horizontal lines represent normal values. Data are represented as mean±SD, **p<0.01, ***p<0.001.

FIGS. 2A-2H are graphs and images showing that transgenic Fgfr3^{ach/+} mice preferentially develop visceral obesity that is prevented upon sFGFR3 treatment (SEQ ID NO: 1). FIG. 2A is a graph showing body weight of vehicle-treated WT and Fgfr3^{ach/+} mice and sFGFR3 treated Fgfr3^{ach/+} mice after 10 weeks of ND or HFD challenge. FIG. 2B are images and a graph showing the abdominal lean:fat ratio. FIG. 2C is a graph showing epididymal adipose tissue (eAT) weight. FIG. 2D is a graph showing subcutaneous adipose tissue (scAT) weight per gram of body weight. FIG. 2E is a graph showing scAT adipocyte area.

FIG. 2F is a graph showing eAT adipocyte area. FIG. 2G is a graph showing scat scattering of adipocytes according to their diameter. Data are presented as mean+/− standard deviation (n=8-10 mice to each group). Data followed normal distribution. *p<0.05, **p<0.01, ***p<0.001 versus vehicle-treated WT,^{#} p<0.05,^{##} p<0.01 versus vehicle-treated Fgfr3^{ach/+}; Student's t test. FIG. 2H is a graph showing eAT scattering of adipocytes according to their diameter. Data are presented as mean+/− standard deviation (n=8-10 mice to each group). Data followed normal distribution. *p<0.05, **p<0.01, ***p<0.001 versus vehicle-treated WT,^{#},^{##} p<0.05, p<0.01 versus vehicle-treated Fgfr3^{ach/+}; Student's t test.

FIGS. 3A-3B are graphs showing that MSCs isolated from untreated or sFGFR3-treated Fgfr3^{ach/+} mice demonstrate preengagement toward adipogenesis with no alteration of the insulin response compared to WT mice. FIG. 3A is a graph showing expression of genes involved in different steps of adipogenesis differentiation (genes listed in Table 1). Expression was normalized to HPRT, RPL6 and RPL13a expression and expressed as percent of change compared to WT. FIG. 3B are graphs showing the results of cells stimulated with 50 nM of insulin for 0, 5, 15 or 30 min or with 0, 1, 10, 50 or 100 nM of insulin during 5 min. P-Erk1/2 expression, normalized to Erk1/2 total expression, was expressed as normalized value to WT. Data are represented as mean±SD. Data followed normal distribution. *p<0.05, **p<0.01, ***p<0.001 versus vehicle-treated WT,^{#} p<0.05,^{##} p<0.01 versus vehicle-treated Fgfr3^{ach/+}. Two-way ANOVA with Tukey's multiple test.

FIGS. 4A-4E are graphs and microscopy images showing that glucose metabolism is altered in transgenic Fgfr3ach/+ mice and restored with sFGFR3 treatment. FIG. 4A is a graph showing fasting glycemia and insulinemia of mice following 10 weeks of ND. FIG. 4B is a graph showing fasting glycemia and insulinemia of mice following 10 weeks of HFD. FIG. 4C shows graphs of the results of a HFD glucose tolerance test; glucose levels were normalized to the value of time −15 min and area under the curve corresponding to each group of mice. FIG. 4D first shows microscopy of mice pancreas insulin content (immunohistochemistry of paraffin-embedded sections, red: insulin; green: glucose; blue: DAPI staining). FIG. 4D also shows a graph of mice pancreas insulin content, mean of pancreas islets normalized to total surface and mean of islets number in each group under an HFD condition. FIG. 4E shows microscopy of liver H&E and PAS staining under HFD condition. FIG. 4F shows microscopy of H&E staining of hepatic nodules. Data are represented as mean±SD (n=8-10 mice to each group). Data followed normal distribution. **p<0.01, ***p<0.001 versus vehicle-treated WT,^{#} p<0.05,^{##} p<0.01 versus vehicle-treated Fgfr3ach/+; Student's t-test.

FIGS. 5A-5D are graphs showing that untreated transgenic Fgfr3^{ach/+} mice draw essentially all their energy from lipids. FIG. 5A shows basal respiratory quotient (RQ=VCO2/VO2) during night or day fasting and feeding periods following 10 weeks of ND challenge. FIG. 5B shows basal carbohydrate and lipid oxidation in WT and Fgfr3^{ach/+} ND challenged mice. FIG. 5C shows basal respiratory quotient (RQ=VCO2/VO2) during night or day fasting and feeding periods following 10 weeks of HFD challenge. FIG. 5D shows basal carbohydrate and lipid oxidation in WT and Fgfr3^{ach/+} HFD challenged mice. Data are represented as mean±SD (n=8-10 mice to each group). Data followed normal distribution. **p<0.01, ***p<0.001 versus vehicle-treated WT,^{#} p<0.05,^{##} p<0.01 versus vehicle-treated Fgfr3^{ach/+}; Student's t test.

FIGS. 6A-6B are graphs showing circulating adipokines studied in the serum of untreated or sFGFR3-treated Fgfr3^{ach/+} mice. FIG. 6A is a graph showing results of mice challenged with a ND. FIG. 6B is a graph showing results of mice challenged with a HFD. Results were expressed as percent of change compared to WT. AgRP, agoutirelated protein; ANGPT-L3, angiopoietin-3; CRP, C-reactive protein; DPPIV, dipeptidyl peptidase V; FGF, fibroblast growth factor; HGF, hepatocyte growth factor; ICAM-1, intercellular adhesion molecule-1; IGF, insulin-like growth factor; IGFBP, insulin-like growth factor binding protein; MCP-1, monocyte chemotactic protein-1; M-CSF, macrophage colonystimulating factor; Pref-1, preadipocyte factor 1; RAGE, receptor for advanced glycation endproducts; RANTES, receptor upon activation, normal T-cell expressed and secreted; RBP4, retinol binding protein; TIMP-1, tissue inhibitor of metalloproteinases; VEGF, vascular endothelial growth factor.

FIGS. 7A-7H are graphs showing that transgenic achondroplasia mice displayed normal energy expenditure, cumulative activity, and cumulative rearing during indirect calorimetry. FIG. 7A shows basal oxygen consumption during night or day fasting and feeding periods in WT and Fgfr3^{ach/+} ND challenged mice. FIG. 7B shows basal carbon dioxide production during night or day fasting and feeding periods for in WT and Fgfr3^{ach/+} ND challenged mice. FIG. 7C shows basal energy expenditure during night or day fasting and feeding periods for in WT and Fgfr3^{ach/+} ND challenged mice. FIG. 7D shows basal cumulative activity and rearing in WT and Fgfr3^{ach/+} ND challenged mice. FIG. 7E shows basal oxygen consumption during night or day fasting and feeding periods in WT and Fgfr3^{ach/+} HFD challenged mice. FIG. 7F shows basal carbon dioxide production during night or day fasting and feeding periods for in WT and Fgfr3^{ach/+} HFD challenged mice. FIG. 7G shows basal energy expenditure during night or day fasting and feeding periods for in WT and Fgfr3^{ach/+} HFD challenged mice. FIG. 7H shows basal cumulative activity and rearing in WT and Fgfr3^{ach/+} HFD challenged mice. Data are represented as mean±SD (n=8-10 mice to each group). Data followed normal distribution. **p<0.01, ***p<0.001 versus vehicle-treated WT,^{###} p<0.001 versus vehicle-treated Fgfr3^{ach/+}; Student's t test.

### DETAILED DESCRIPTION OF THE INVENTION

We have discovered that soluble fibroblast growth factor receptor 3 (sFGFR3) polypeptides and polynucleotides encoding the sFGFR3 polypeptides, and variants thereof, can be used to treat abnormal visceral fat deposition in a patient (e.g., a human, particularly a fetus, a neonate, a child, an adolescent, and an adult) in need thereof. In particular, sFGFR3 polypeptides that can be used in the methods of treatment described herein include those having an amino acid sequence of any one of SEQ ID NOs: 1-7 and variants thereof having at least 85% sequence identity thereto. Polynucleotides encoding the sFGFR polypeptides or cells containing the polynucleotides can also be administered in the methods of treatment.

### Methods of Treatment

Provided herein are methods for treating a patient with abnormal visceral fat deposition and the conditions associated with abnormal visceral fat deposition. In particular, the patient can have an elevated body mass index, sagittal diameter, android:gynoid fat ratio, fat mass index, and visceral fat area. The patient may also have a skeletal growth retardation syndrome, e.g., achondroplasia, thanatophoric dysplasia type I (TDI), thanatophoric dysplasia type II (TDII), severe achondroplasia with developmental delay and Acanthosis nigricans (SADDAN), hypochondroplasia, and craniosynostosis syndromes (e.g., Muenke syndrome, Crouzon syndrome, and Crouzonodermoskeletal syndrome, camptodactyly, tall stature, and hearing loss syndrome (CATSHL). It may be the case that the patient has a skeletal growth retardation syndrome and a condition associated with abnormal visceral fat deposition (e.g., atherosclerosis, hypertension, myocardial infarction, dyslipidemia, sleep apnea, restrictive lung disease, asthma, dementia, dysregulation of insulin (e.g., insulin resistance), dysregulation of glucose metabolism, infertility, menstrual irregularities, stroke, and dementia.

Alternatively, the patient may be one that does not have a skeletal growth retardation syndrome, but does have a condition that leads to abnormal visceral fat deposition (e.g., obesity, polycystic ovary syndrome, and hypercortisolism (e.g., Cushing's disease)). For example, the patient may have abnormal visceral fat deposition and a condition associated with abnormal visceral fat deposition (e.g., atherosclerosis, hypertension, myocardial infarction, dyslipidemia, sleep apnea, restrictive lung disease, asthma, dementia, dysregulation of insulin (e.g., insulin resistance), dysregulation of glucose metabolism, infertility, menstrual irregularities, stroke, and dementia). The method may also involve administration of a sFGFR3 to treat a patient with aberrant signaling of FGF10, such as a patient with Cushing's disease caused by pituitary gland dysfunction.

The patient may also be characterized as having visceral fat deposition associated with or surrounding one or more of the following organs: the heart, liver, spleen, kidneys, pancreas, intestines, reproductive organs, and gall bladder

The method involves administering an sFGFR3 polypeptide of the invention, e.g., those described herein, to the patient having abnormal visceral fat deposition. The patient may be a fetus, a neonate, an infant, a child, an adolescent, or an adult at risk for developing abnormal abdominal fat deposition. The patient may also have a skeletal growth retardation syndrome (e.g., achondroplasia), obesity, hypercortisolism (e.g., Cushing's disease), or polycystic ovary syndrome. The patient (e.g., a human) can be treated before signs and symptoms of abnormal visceral fat deposition develop. In particular, patients that can be treated with a sFGFR3 polypeptide of the invention described herein are those exhibiting symptoms including, but not limited to, abnormal fat mass index, abnormal area of visceral fat, elevated BMI, increased waist circumference, increased sagittal diameter, and increased android:gynoid fat ratio. Furthermore, patients that can be treated with a sFGFR3 polypeptide have abnormal visceral fat distribution and conditions associated with abnormal visceral fat deposition (e.g., metabolic, cardiovascular, pulmonary, reproductive or neurologic diseases). Furthermore, treatment with an sFGFR3 polypeptide can result in an improvement in one or more of the aforementioned symptoms related to abnormal visceral fat deposition (e.g., relative to an untreated patient). The patient (e.g., a human) can be diagnosed with abnormal visceral fat deposition, such as one with skeletal growth retardation syndrome (e.g., achondroplasia), obesity, hypercortisolism (e.g., Cushing's disease), and polycystic ovary syndrome, before administration of an sFGFR3 polypeptide. Additionally, the patient having abnormal visceral fat deposition can be one that has not previously been treated with an sFGFR3 polypeptide.

A patient that can be treated with an sFGFR3 polypeptide also includes a patient with, or at risk of developing, diabetes, such as an obese patient. Treatment of this patient may involve locally administering the soluble FGFR3 to the pancreas in order to treat or prevent diabetes development.

Soluble Fibroblast Growth Factor Receptor 3 (sFGFR3) Polypeptides

Soluble FGFR3 polypeptides and variants thereof can be used in the methods of treating a patient having abnormal visceral fat deposition. The sFGFR3 polypeptide can include at least 50 consecutive amino acids of an extracellular domain (ECD) of a naturally occurring fibroblast growth factor receptor 3 (FGFR3) polypeptide (e.g., the FGFR3 polypeptide having the sequence set forth in Genbank Accession No. NP_000133; see also SEQ ID NO: 8). In particular, the sFGFR3 polypeptide may include 100-370 consecutive amino acids (e.g., fewer than 350 consecutive amino acids) of an ECD of a naturally occurring fibroblast growth factor receptor 3 (FGFR3) polypeptide. The sFGFR3 polypeptide may also have an Ig-like C2-type domain 1, 2, and/or 3 of a naturally occurring FGFR3 polypeptide.

The sFGFR3 polypeptide may have, or may lack, a signal peptide (e.g., a signal peptide of an FGFR3 polypeptide, such as that corresponding to SEQ ID NO: 21; in particular, the sFGFR3 is a mature polypeptide lacking the signal peptide, which is cleaved during expression and secretion from the cell). The sFGFR3 polypeptide also lacks a transmembrane domain™, such as the TM of a naturally occurring FGFR3 polypeptide.

The sFGFR3 polypeptides may also contain all or a portion of an intracellular domain (ICD) of an FGFR3 polypeptide. For example, the sFGFR3 polypeptide may have 400 consecutive amino acids or fewer (e.g., between 5 and 399 consecutive amino acids, such as 175, 150, 125, 100, 75, 50, 40, 30, 20, 15, or fewer consecutive amino acids) of an ICD of a naturally-occurring FGFR3 polypeptide. The ICD of the sFGFR3 polypeptide may also lack a tyrosine kinase domain of a naturally-occurring FGFR3 polypeptide. Alternatively, the sFGFR3 polypeptide may lack any amino acids of an ICD of a naturally-occurring FGFR3 polypeptide (e.g., the FGFR3 polypeptide of SEQ ID NO: 8).

The sFGFR3 polypeptide may also have an amino acid sequence with at least 90%, 92%, 95%, 97%, or 99% sequence identity to, or the sequence of, amino acids 401 to 413 of SEQ ID NO: 8.

An sFGFR3 polypeptide for use in the methods described herein may be fewer than 475, 450, 425, 400, 375, 350, 300, 250, 200, 150, or 100 amino acids in length and/or may have an amino acid sequence with at least 85% sequence identity (e.g., 86%-100% sequence identity, such as 86%, 87%, 88%, 89%, 90%, 91%, 92%, 93%, 94%, 95%, 96%, 97%, 98%, or 99% sequence identity) to amino acids residues 1 to 280 of SEQ ID NO: 8. The sFGFR polypeptide may also be one with an amino acid sequence having at least 85% sequence identity (e.g., 86%-100% sequence identity, such as 86%, 87%, 88%, 89%, 90%, 91%, 92%, 93%, 94%, 95%, 96%, 97%, 98%, or 99% sequence identity) to the amino acid sequence of any one of SEQ ID NOs: 1-7. In particular, the sFGFR3 has the amino acid sequence of SEQ ID NO: 5 or 6 (e.g., the amino acid sequence of SEQ ID NO: 5). The sFGFR3 polypeptide may also have the sequence of SEQ ID NO: 6, except that the residue at position 253 is an alanine, glycine, proline, or threonine.

sFGFR3 polypeptide variants that can be administered in the methods also include fragments of the amino acid sequence of any one of SEQ ID NOs: 1-8 (e.g., at least amino acids 1 to 200, 1 to 205, 1 to 210, 1 to 215, 1 to 220, 1 to 225, 1 to 235, 1 to 230, 1 to 240, 1 to 245, 1 to 250, 1 to 253, 1 to 255, 1 to 260, 1 to 265, 1 to 275, 1 to 280, 1 to 285, 1 to 290, or 1 to 300, of SEQ ID NO: 8) or polypeptides having at least 50% sequence identity (e.g., 55%, 60%, 65%, 70%, 75%, 80%, 85%, 86%, 87%, 88%, 89%, 90%, 91%, 92%, 93%, 94%, 95%, 96%, 97%, 98%, 99%, or more sequence identity) to any one of SEQ ID NOs: 1-8 (and that, e.g., lack a signal peptide and TM domain).

The sFGFR3 polypeptides of the invention can also be characterized as binding to a fibroblast growth factor (FGF). In particular, the FGF is selected from the group consisting of fibroblast growth factor 1 (FGF1; SEQ ID NO: 26), fibroblast growth factor 2 (FGF2; SEQ ID NO: 27), fibroblast growth factor 9 (FGF9; SEQ ID NO: 28), fibroblast growth fact 10 (FGF10; SEQ ID NO: 40), fibroblast growth factor 18 (FGF18; SEQ ID NO: 29), fibroblast growth factor 19 (FGF19; SEQ ID NO: 30), fibroblast growth factor 21 (FGF21; SEQ ID NO: 31), and fibroblast growth factor 23 (FGF23; SEQ ID NO: 41). The binding is characterized by an equilibrium dissociation constant (K_{d}) of about 0.2 nM to about 20 nM (e.g., a K_{d} of about 1 nM to about 10 nM, wherein optionally the K_{d} is about 1 nm, about 2 nm, about 3 nm, about 4 nm, about 5 nm, about 6 nm, about 7 nm, about 8 nm, about 9 nm, or about 10 nm).

Given the results described herein, the invention is not limited to a particular sFGFR3 polypeptide or variant thereof. In addition to the exemplary sFGFR3 polypeptides and variants thereof discussed above, any sFGFR3 polypeptide that binds one or more FGFs with a similar binding affinity as the sFGFR3 polypeptides having the amino acids sequence of SEQ ID NOs: 1-7 are also envisioned as being useful for treating abnormal visceral fat deposition in a subject in need thereof. The sFGFR3 polypeptides can be, for example, fragments of FGFR3 isoform 2 lacking exons 8 and 9 encoding the C-terminal half of the IgG3 domain and exon 10 including the transmembrane domain (e.g., fragments of the amino acid sequence of SEQ ID NO: 8), corresponding to fragments of FGFR3 transcript variant 2 (Accession No. NM_022965).

As noted above, an sFGFR3 polypeptide for use in the methods of the invention can include a signal peptide at the N-terminal position. An exemplary signal peptide can include, but is not limited to, amino acids 1 to 22 of SEQ ID NO: 21 (e.g., MGAPACALALCVAVAIVAGASS). Accordingly, the sFGFR3 polypeptides include both secreted forms, which lack the N-terminal signal peptide, and non-secreted forms, which include the N-terminal signal peptide. For instance, a secreted sFGFR3 polypeptide can include the amino acid sequence of any one of SEQ ID NOs: 1-7, but without an N-terminal signal peptide (e.g., the sequence of SEQ ID NO: 21). Alternatively, the sFGFR3 polypeptide (e.g., a polypeptide having the amino acid sequence of any one of SEQ ID NOs: 1-7) does include a signal peptide, such as the amino acid sequence of SEQ ID NO: 21. One skilled in the art will appreciate that the position of the N-terminal signal peptide will vary in different sFGFR3 polypeptides and can include, for example, the first 5, 8, 10, 11, 12, 13, 14, 15, 16, 17, 18, 19, 20, 21, 22, 23, 24, 25, 27, 30, or more amino acid residues on the N-terminus of the polypeptide. One of skill in the art can predict the position of a signal sequence cleavage site, e.g., by an appropriate computer algorithm such as that described in Bendtsen et al. (J. Mol. Biol. 340(4):783-795, 2004) and available on the Web at cbs.dtu.dk/services/SignalP/.

Additionally, sFGFR3 polypeptides of the invention can be glycosylated. In particular, a sFGFR3 polypeptide can be altered to increase or decrease the extent to which the sFGFR3 polypeptide is glycosylated. Addition or deletion of glycosylation sites to an sFGFR3 polypeptide can be accomplished by altering the amino acid sequence such that one or more glycosylation sites is created or removed. For example, N-linked glycosylation, in which an oligosaccharide is attached to the amide nitrogen of an asparagine residue, can occur at position Asn76, Asn148, Asn169, Asn 203, Asn240, Asn272, and/or Asn 294 of the amino acid sequence of SEQ ID NO: 5 or 6 and variants thereof. One or more of these Asn residues can also be substituted to remove the glycosylation site. For instance, O-linked glycosylation, in which an oligosaccharide is attached to an oxygen atom of an amino acid residue, can occur at position Ser109, Thr126, Ser199, Ser274, Thr281, Ser298, Ser299, and/or Thr301 of the amino acid sequence of SEQ ID NO: 5 or 6 and variants thereof. Additionally, O-linked glycosylation can occur at a serine residue within the sFGFR3. One or more of these Ser or Thr residues can also be substituted to remove the glycosylation site.

sFGFR3 Fusion Polypeptides

sFGFR3 polypeptides of the invention (e.g., sFGFR3 polypeptides having the amino acid sequence of any one of SEQ ID NOs: 1-7 or a variant thereof having at least 85% sequence identity thereto (e.g., 86%-100% sequence identity, such as 86%, 87%, 88%, 89%, 90%, 91%, 92%, 93%, 94%, 95%, 96%, 97%, 98%, or 99% sequence identity thereto) can be fused to a functional domain from a heterologous polypeptide (e.g., a fragment crystallizable region (Fc region; such as a polypeptide having the amino acid sequence of SEQ ID NOs: 35 and 36) or human serum albumin (HSA; such as a polypeptide having the amino acid sequence of SEQ ID NO: 37)) to provide a sFGFR3 fusion polypeptide. Optionally, a flexible linker, can be included between the sFGFR3 polypeptide and the heterologous polypeptide (e.g., an Fc region or HSA), such as a serine or glycine-rich sequence (e.g., a poly-glycine or a poly-glycine/serine linker, such as SEQ ID NOs: 38 and 39).

For example, the sFGFR3 polypeptides and variants thereof can be a fusion polypeptide including, e.g., an Fc region of an immunoglobulin at the N-terminal or C-terminal domain. In particular, useful Fc regions can include the Fc fragment of any immunoglobulin molecule, including IgG, IgM, IgA, IgD, or IgE and their various subclasses (e.g., IgG-1, IgG-2, IgG-3, IgG-4, IgA-1, IgA-2) from any mammal (e.g., a human). For instance, the Fc fragment human IgG-1 (SEQ ID NO: 35) or a variant of human IgG-1, such as a variant including a substitution of asparagine at position 297 of SEQ ID NO: 35 with alanine (e.g., a polypeptide having the amino acid sequence of SEQ ID NO: 36). The Fc fragments of the invention can include, for example, the CH2 and CH3 domains of the heavy chain and any portion of the hinge region. The sFGFR3 fusion polypeptides of the invention can also include, e.g., a monomeric Fc, such as a CH2 or CH3 domain. The Fc region may optionally be glycosylated at any appropriate one or more amino acid residues known to those skilled in the art. An Fc fragment as described herein may have 1, 2, 3, 4, 5, 6, 7, 8, 9, 10, 11, 12, 13, 14, 15, 16, 17, 18, 19, 20, 25, 30, 35, 40, 50, or more additions, deletions, or substitutions relative to any of the Fc fragments described herein.

Additionally, the sFGFR3 polypeptides can be conjugated to other molecules at the N-terminal or C-terminal domain for the purpose of improving the solubility and stability of the protein in aqueous solution. Examples of such molecules include human serum albumin (HSA), PEG, PSA, and bovine serum albumin (BSA). For instance, the sFGFR3 polypeptides can be conjugated to human HSA (e.g., a polypeptide having the amino acid sequence of SEQ ID NO: 37) or a fragment thereof.

The sFGFR3 fusion polypeptides can include a peptide linker region between the sFGFR3 polypeptide and the heterologous polypeptide (e.g., an Fc region or HSA). The linker region may be of any sequence and length that allows the sFGFR3 to remain biologically active, e.g., not sterically hindered. Exemplary linker lengths are between 1 and 200 amino acid residues, e.g., 1-5, 6-10, 11-15, 16-20, 21-25, 26-30, 31-35, 36-40, 41-45, 46-50, 51-55, 56-60, 61-65, 66-70, 71-75, 76-80, 81-85, 86-90, 91-95, 96-100, 101-110, 111-120, 121-130, 131-140, 141-150, 151-160, 161-170, 171-180, 181-190, or 191-200 amino acid residues. For instance, linkers include or consist of flexible portions, e.g., regions without significant fixed secondary or tertiary structure. Preferred ranges are 5 to 25 and 10 to 20 amino acids in length. Such flexibility is generally increased if the amino acids are small and do not have bulky side chains that impede rotation or bending of the amino acid chain. Thus, preferably the peptide linker of the present invention has an increased content of small amino acids, in particular of glycines, alanines, serines, threonines, leucines and isoleucines.

Exemplary flexible linkers are glycine-rich linkers, e.g., containing at least 50%, 60%, 65%, 70%, 75%, 80%, 85%, 90%, 95%, or even 100% glycine residues. Linkers may also contain, e.g., serine-rich linkers, e.g., containing at least 50%, 60%, 65%, 70%, 75%, 80%, 85%, 90%, 95%, or even 100% serine residues. In some cases, the amino acid sequence of a linker consists only of glycine and serine residues. For example, the linker can be the amino acid sequence of GGGGAGGGG (SEQ ID NO: 38) or GGGGSGGGGSGGGGS (SEQ ID NO: 39). A linker can optionally be glycosylated at any appropriate one or more amino acid residues. The linker can also be absent, in which the sFGFR3 polypeptide and the heterologous polypeptide (e.g., an Fc region or HSA) are fused together directly, with no intervening residues.

Polynucleotides Encoding the sFGFR3 Polypeptides

Polynucleotides encoding the sFGFR3 polypeptides can be used to treat a patient having abnormal visceral fat deposition in a patient (e.g., a human, such as a fetus, a neonate, an infant, a child, an adolescent, or an adult). For example, the polynucleotide can have the nucleic acid sequence of any one of SEQ ID NOs: 10-18 or a variant thereof having at least 85% sequence identity (e.g., 86%, 87%, 88%, 89%, 90%, 91%, 92%, 93%, 94%, 95%, 96%, 97%, 98%, 99%, or more sequence identity) to the nucleic acid sequence of any one of SEQ ID NOs: 10-18. Additionally, the polynucleotide can have the nucleic acid sequence of SEQ ID NO: 14 or 15 or a variant having at least 85% sequence identity (e.g., 86%, 87%, 88%, 89%, 90%, 91%, 92%, 93%, 94%, 95%, 96%, 97%, 98%, 99%, or more sequence identity) to the nucleic acid sequence of SEQ ID NO: 14 or 15.

Also featured are polynucleotides encoding sFGFR3 fusion polypeptides (e.g., a sFGFR3 polypeptide fused to a heterologous polypeptide, such as a Fc region or HSA) and polynucleotides encoding sFGFR3 polypeptides without a signal peptide (e.g., polypeptides having the amino acid sequence of any one of SEQ ID NOs: 1-7) or with a signal peptide (e.g., polypeptides having the amino acid sequence of any one of SEQ ID NOs: 1-7. Additionally, the polynucleotides can have one or more mutations to alter any of the glycosylation sites described herein or known to be present in the polypeptide.

Optionally, the polynucleotides of the invention can be codon optimized to alter the codons in the nucleic acid, in particular to reflect the typical codon usage of the host organism (e.g., a human) without altering the sFGFR3 polypeptide encoded by the nucleic acid sequence of the polynucleotide. Codon-optimized polynucleotides (e.g., a polynucleotide having the nucleic acid sequence of SEQ ID NO: 14 or 16) can, e.g., facilitate genetic manipulations by decreasing the GC content and/or for expression in a host cell (e.g., a HEK 293 cell or a CHO cell). Codon-optimization can be performed by the skilled person, e.g. by using online tools such as the JAVA Codon Adaption Tool (www.jcat.de) or Integrated DNA Technologies Tool (www.eu.idtdna.com/CodonOpt) by simply entering the nucleic acid sequence of the polynucleotide and the host organism for which the codons are to be optimized. The codon usage of different organisms is available in online databases, for example, www.kazusa.or.jp/codon.

Host Cells for Expression of the sFGFR3 Polypeptides

Mammalian cells can be used as host cells for expression of the sFGFR3 polypeptide (e.g., a polypeptide having the amino acid sequence of any one of SEQ ID NOs: 1-7 and variants thereof). Exemplary mammalian cell types useful in the methods include, but are not limited to, human embryonic kidney (HEK; e.g., HEK 293) cells, Chinese Hamster Ovary (CHO) cells, L cells, C127 cells, 3T3 cells, BHK cells, COS-7 cells, HeLa cells, PC3 cells, Vero cells, MC3T3 cells, NS0 cells, Sp2/0 cells, VERY cells, BHK, MDCK cells, W138 cells, BT483 cells, Hs578T cells, HTB2 cells, BT20 cells, T47D cells, NS0 cells, CRL7O3O cells, and HsS78Bst cells, or any other suitable mammalian host cell known in the art. Alternatively, E. coli cells can be used as host cells for expression of the sFGFR3 polypeptides. Examples of E. coli strains include, but are not limited to, E. coli 294 (ATCC® 31,446), E. coli A 1776 (ATCC° 31,537, E. coli BL21 (DE3) (ATCC® BAA-1025), E. coli RV308 (ATCC® 31,608), or any other suitable E. coli strain known in the art.

Vectors Including Polynucleotides Encoding the sFGFR3 Polypeptides

Also featured are recombinant vectors including any one or more of the polynucleotides described above (e.g., a polynucleotide encoding a polypeptide having the amino acid sequence of any one of SEQ ID NOs: 1-7 and variants thereof). The vectors of the invention can be used to deliver a polynucleotide encoding a sFGFR3 polypeptide of the invention and variants thereof, which can include mammalian, viral, and bacterial expression vectors. For example, the vectors can be plasmids, artificial chromosomes (e.g. BAG, PAC, and YAC), and virus or phage vectors, and may optionally include a promoter, enhancer, or regulator for the expression of the polynucleotide. The vectors can also contain one or more selectable marker genes, such as an ampicillin, neomycin, and/or kanamycin resistance gene in the case of a bacterial plasmid or a resistance gene for a fungal vector. Vectors can be used in vitro for the production of DNA or RNA or used to transfect or transform a host cell, such as a mammalian host cell for the production of a sFGFR3 polypeptide encoded by the vector. The vectors can also be adapted to be used in vivo in a method of gene therapy.

Exemplary viral vectors that can be used to deliver a polynucleotide encoding a sFGFR3 polypeptide of the invention (e.g., a polypeptide having the amino acid sequence of any one of SEQ ID NOs: 1-7 and variants thereof) include a retrovirus, adenovirus (e.g., Ad2, Ad5, Ad11, Ad12, Ad24, Ad26, Ad34, Ad35, Ad40, Ad48, Ad49, Ad50, and Pan9 (also known as AdC68)), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding sFGFR3 polypeptides include Norwalk virus, togavirus, flavivirus, reoviruses, papovavirus, hepadnavirus, and hepatitis virus. Examples of retroviruses include avian leukosis-sarcoma, mammalian C-type, B-type viruses, D-type viruses, HTLV-BLV group, lentivirus, and spumavirus (Coffin, J. M., Retroviridae: The viruses and their replication, In Fundamental Virology, Third Edition, B. N. Fields, et al., Eds., Lippincott-Raven Publishers, Philadelphia, 1996).

### Methods of Production

Polynucleotides encoding sFGFR3 polypeptides of the invention (e.g., a polypeptide having the amino acid sequence of any one of SEQ ID NOs: 1-7 and variants thereof) can be produced by any method known in the art. For instance, a polynucleotide is generated using molecular cloning methods and is placed within a vector, such as a plasmid, an artificial chromosome, a viral vector, or a phage vector. The vector is used to transform the polynucleotide into a host cell appropriate for the expression of the sFGFR3 polypeptide.

### Nucleic Acid Vector Construction and Host Cells

The sFGFR3 polypeptides of the invention (e.g., a polypeptide having the amino acid sequence of any one of SEQ ID NOs: 1-7 and variants thereof) can be produced from a host cell. The polynucleotides (e.g., polynucleotides having the nucleic acid sequence of SEQ ID NO: 14 or 16 and variants thereof) encoding sFGFR3 polypeptides can be included in vectors that can be introduced into the host cell by conventional techniques known in the art (e.g., transformation, transfection, electroporation, calcium phosphate precipitation, direct microinjection, or infection). The choice of vector depends in part on the host cells to be used. Generally, host cells are of either prokaryotic (e.g., bacterial) or eukaryotic (e.g., mammalian) origin.

A polynucleotide encoding an sFGFR3 polypeptide of the invention (e.g., a polypeptide having the amino acid sequence of any one of SEQ ID NOs: 1-7 and variants thereof) can be prepared by a variety of methods known in the art. These methods include, but are not limited to, oligonucleotide-mediated (or site-directed) mutagenesis and PCR mutagenesis. A polynucleotide encoding an sFGFR3 polypeptide can be obtained using standard techniques, e.g., gene synthesis. Alternatively, a polynucleotide encoding a wild-type sFGFR3 polypeptide (e.g., a polypeptide having the amino acid sequence of SEQ ID NO: 8) can be mutated to contain specific amino acid substitutions using standard techniques in the art, e.g., QuikChange™ mutagenesis. Polynucleotides encoding an sFGFR3 polypeptide can be synthesized using, e.g., a nucleotide synthesizer or PCR techniques.

Polynucleotides encoding sFGFR3 polypeptide of the invention (e.g., a polypeptide having the amino acid sequence of any one of SEQ ID NOs: 1-7 and variants thereof) can be inserted into a vector capable of replicating and expressing the polynucleotide in prokaryotic or eukaryotic host cells. Exemplary vectors useful in the methods can include, but are not limited to, a plasmid, an artificial chromosome, a viral vector, and a phage vector. For example, a viral vector can include the viral vectors described above, such as a retroviral vector, adenoviral vector, or poxviral vector (e.g., vaccinia viral vector, such as Modified Vaccinia Ankara (MVA)), adeno-associated viral vector, and alphaviral vector)) containing the nucleic acid sequence of a polynucleotide encoding the sFGFR3 polypeptide. Each vector can contain various components that may be adjusted and optimized for compatibility with the particular host cell. For example, the vector components may include, but are not limited to, an origin of replication, a selection marker gene, a promoter, a ribosome binding site, a signal sequence, the nucleic acid sequence of the polynucleotide encoding the sFGFR3 polypeptide, and/or a transcription termination sequence.

The above-described vectors may be introduced into appropriate host cells (e.g., HEK 293 cells or CHO cells, or into a host cell of a subject) using conventional techniques in the art, e.g., transformation, transfection, electroporation, calcium phosphate precipitation, and direct microinjection. Once the vectors are introduced into host cells for the production of an sFGFR3 polypeptide of the invention, the host cells are cultured in conventional nutrient media modified as appropriate for inducing promoters, selecting transformants, or amplifying the polynucleotides encoding the sFGFR3 polypeptide. Methods for expression of therapeutic proteins, such as sFGFR3 polypeptides, are known in the art, see, for example, Paulina Balbas, Argelia Lorence (eds.) Recombinant Gene Expression: Reviews and Protocols (Methods in Molecular Biology), Humana Press' 2nd ed. 2004 (Jul. 20, 2004) and Vladimir Voynov and Justin A. Caravella (eds.) Therapeutic Proteins: Methods and Protocols (Methods in Molecular Biology) Humana Press' 2nd ed. 2012 (Jun. 28, 2012), each of which is hereby incorporated by reference in its entirety.

sFGFR3 Polypeptide Production, Recovery, and Purification

Host cells (e.g., HEK 293 cells or CHO cells) used to produce the sFGFR3 polypeptide of the invention (e.g., a polypeptide having the amino acid sequence of any one of SEQ ID NOs: 1-7 and variants thereof) can be grown in media known in the art and suitable for culturing of the selected host cells. Examples of suitable media for mammalian host cells include Minimal Essential Medium (MEM), Dulbecco's Modified Eagle's Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin. Host cells are cultured at suitable temperatures, such as from about 20° C. to about 39° C., e.g., from 25° C. to about 37° C., preferably 37° C., and CO₂ levels, such as 5 to 10% (preferably 8%). The pH of the medium is generally from about 6.8 to 7.4, e.g., 7.0, depending mainly on the host organism. If an inducible promoter is used in the expression vector, sFGFR3 polypeptide expression is induced under conditions suitable for the activation of the promoter.

An sFGFR3 polypeptide of the invention (e.g., a polypeptide having the amino acid sequence of any one of SEQ ID NOs: 1-7 and variants thereof) can be recovered from the supernatant of the host cell. Alternatively, the sFGFR3 polypeptide can be recovered by disrupting the host cell (e.g., using osmotic shock, sonication, or lysis), followed by centrifugation or filtration to remove the sFGFR3 polypeptide. Upon recovery of the sFGFR3 polypeptide, the sFGFR3 polypeptide can then be further purified. An sFGFR3 polypeptide can be purified by any method known in the art of protein purification, such as protein A affinity, other chromatography (e.g., ion exchange, affinity, and size-exclusion column chromatography), centrifugation, differential solubility, or by any other standard technique for the purification of proteins (see Process Scale Purification of Antibodies, Uwe Gottschalk (ed.) John Wiley & Sons, Inc., 2009, hereby incorporated by reference in its entirety).

Optionally, the sFGFR3 polypeptide of the invention (e.g., a polypeptide having the amino acid sequence of any one of SEQ ID NOs: 1-7 and variants thereof) can be conjugated to a detectable label for purification. Examples of suitable labels for use in purification of the sFGFR3 polypeptides include, but are not limited to, a protein tag, a fluorophore, a chromophore, a radiolabel, a metal colloid, an enzyme, or a chemiluminescent, or bioluminescent molecule. In particular, protein tags that are useful for purification of the sFGFR3 polypeptides can include, but are not limited to, chromatography tags (e.g., peptide tags consisting of polyanionic amino acids, such as a FLAG-tag, or a hemagglutin in “HA” tag), affinity tags (e.g., a poly(His) tag, chitin binding protein (CBP), maltose binding protein (MBP), or glutathione-S-transferase (GST)), solubilization tags (e.g., thioredoxin (TRX) and poly(NANP)), epitope tags (e.g., V5-tag, Myc-tag, and HA-tag), or fluorescence tags (e.g., GFP, GFP variants, RFP, and RFP variants).

### Methods of Diagnosis

A patient with abnormal visceral fat deposition can be identified as being in need of treatment by one of many techniques known in the art. There are two categories of techniques: anthropometric techniques and imaging. Anthropometric techniques rely on measurements based on tape measure and scale. Imaging techniques rely on attenuation of x-ray beams as they travel through a patient or on the magnetic properties of a patient's hydrogen nuclei.

A commonly used anthropometric technique that categorizes patients as normal weight, overweight, or obese, is body mass index (BMI). BMI is calculated by dividing the a patient's weight (in kg) by the square of the patient's height (in m). The cutoff indicating obesity in adults is different from thresholds in children. For adults, obesity is considered to be present when the BMI is 30 kg/m² or greater (see, e.g., Nuttall, Nutr. Today 50(3):117-28, 2015); for children, the threshold for obesity varies based on age and height (see, e.g., van der Sluis et al., Arch. Dis. Child. 87(4):341-347, 2002). The standard tables for BMI in children have limitations when achondroplastic children are considered (see, e.g., Hoover-Fong et al., Am. J. Clin. Nutr. 88, 364-371, 2008).

Among anthropometric techniques, BMI does not distinguish between subcutaneous and visceral fat deposition. As a result, abnormal abdominal visceral fat is better identified using waist circumference (measured just above the palpated top of the iliac crest bone), sagittal diameter (the anteroposterior distance from the small of the back to the anterior abdomen), and the android:gynoid fat ratio (the waist circumference divided by the hip circumference).

The cutoff for abnormal waist circumference is, for adult females, greater than 83 cm, and for adult men greater than 90 cm (see, e.g., Zhu et al., Am. J. Clin. Nutr. 76(4):743-749, 2002). For sagittal diameters, the cutoff for women is greater than 20.1 cm, and for men, greater than 23.1 cm; see, e.g., Pimentel et al, Nutr. Hosp. 25(4):656-61, 2010. Finally, for the android:gynoid fat ratio, the cutoff for women is greater than 0.85, while for men it is greater than 0.9; see, e.g., Price et al, Am. J. Clin. Nutr. 84(2):449-460, 2006).

One imaging technique that relies on x-rays is dual-energy x-ray absorptiometry (DXA), a method in which two x-ray beams of differing energy are used to determine fat (as opposed to lean) mass, and from this determination, a measure called fat mass index (measured fat mass divided by square of height) is derived. The cutoff for abnormal fat deposition in women is greater than 13, and the cutoff for abnormal fat deposition in men is greater than 9; see, e.g. Kelly et al, PLOS One, 4(9): 2009).

Both CT (using x-ray) and MRI (depending on the magnetic properties of the body) can image the abdomen in cross section, and thereby distinguish visceral from subcutaneous fat deposition. With either CT or MRI, the cutoff for abnormal visceral fat deposition in women is 110 cm², and for men it is 132 cm²; see, e.g., Wajchenberg, Endocr. Rev. 21(6):697-738, 2000.

### Methods of Monitoring Therapy

To assess an effect of therapy, a patient treated with an sFGFR3 polypeptide can be followed using a variety of biomarkers of disease state. For a patient treated to reduce or prevent abnormal visceral fat deposition, monitoring techniques include, e.g., determining improvement in BMI, sagittal diameter, android:gynoid ratio, waist circumference, fat mass index, and area of visceral fat on a standardized cross-sectional image of the abdomen. The effect of therapy can also be assessed using one or more biomarkers found in, e.g., a sample from the patient (e.g., a blood, urine, or sputum sample). In the case of a patient treated to prevent or improve metabolic diseases, such as diabetes or fatty liver, blood tests to monitor an effect of therapy include, e.g., assessing the patient sample for a change in glucose and/or insulin levels, an improvement in glucose tolerance testing, and a decrease in elevated levels of alanine transaminase, aspartate am inotransferase, and/or alkaline phosphatase.

If a patient is receiving sFGFR3 polypeptide therapy to treat or reduce the risk of cardiovascular diseases, a blood test can be performed to show improvement in a level of triglycerides, high density lipoproteins, low density lipoproteins, and/or cholesterol. Alternatively, radioisotope stress testing can be done to show an improvement in a patient's exercise tolerance and/or cardiac perfusion.

For patients treated to improve or reduce the risk of dementia, serial neuropsychology testing can be performed.

In cases in which sFGFR3 polypeptide is administered to treat or reduce the risk of infertility or menstrual irregularities, a level of a blood biomarker, such as follicle-stimulating hormone, luteinizing hormone, estradiol, and prolactin, can be measured.

If a patient is suffering or at risk for sleep apnea, a study that measures, e.g., the sleeping patient's oxygen, respiratory rate, heart rate, snoring, and/or body movement can be performed following therapy with an sFGFR3 polypeptide to assess the effect of therapy.

In a patient for whom an sFGFR3 polypeptide is given to treat or prevent restrictive lung disease or asthma, pulmonary function testing using, e.g., spirometry or plethysmography, can be used to show an improvement in lung measures, such as tidal volume, vital capacity, residual volume, and/or forced vital capacity.

In a case where monitoring techniques indicate that a patient may not be responding to the treatment using an sFGFR3 polypeptide, or for which a greater treatment effect may be desired, administration of an sFGFR3 polypeptide may be repeated one or more times or the frequency of administration may be increased.

Administration of sFGFR3 Polypeptides

An sFGFR3 polypeptide can be administered to treat a patient having or at risk of abnormal visceral fat deposition. Examples of sFGFR3 polypeptides include, e.g., sFGFR3 polypeptides having the amino acid sequence of any one of SEQ ID NOs: 1-7 or a variant thereof having at least 85% sequence identity thereto (e.g., 86%-100% sequence identity, such as 86%, 87%, 88%, 89%, 90%, 91%, 92%, 93%, 94%, 95%, 96%, 97%, 98%, or 99% sequence identity thereto). The sFGFR3 polypeptide can be administered by any route known in the art, such as by parenteral administration, enteral administration, or topical administration. In particular, the sFGFR3 polypeptide can be administered to the patient having increased visceral fat deposition) subcutaneously (e.g., by subcutaneous injection), intravenously, intramuscularly, intra-arterially, intrathecally, or intraperitoneally.

An sFGFR3 polypeptide can be administered to a patient (e.g., a human) at a predetermined dosage, such as in an effective amount to treat increased visceral fat deposition without inducing significant toxicity. For example, sFGFR3 polypeptides can be administered to a patient having increased visceral fat deposition in individual doses ranging from about 0.002 mg/kg to about 20 mg/kg (e.g., from 0.002 mg/kg to 20 mg/kg, from 0.01 mg/kg to 2 mg/kg, from 0.2 mg/kg to 20 mg/kg, from 0.01 mg/kg to 10 mg/kg, from 10 mg/kg to 100 mg/kg, from 0.1 mg/kg to 50 mg/kg, 0.5 mg/kg to 20 mg/kg, 1.0 mg/kg to 10 mg/kg, 1.5 mg/kg to 5 mg/kg, or 0.2 mg/kg to 3 mg/kg). In particular, the sFGFR3 polypeptide can be administered in individual doses of, e.g., 0.001 mg/kg to 7 mg/kg, such as 0.3 mg/kg to about 2.5 mg/kg.

Exemplary doses of an sFGFR3 polypeptide of the invention) for administration to a patient (e.g., a human) having increased visceral fat deposition) include, e.g., 0.005, 0.01, 0.02, 0.05, 0.1, 0.2, 0.3, 0.4, 0.5, 0.6, 0.7, 0.8, 0.9, 1, 2, 2.5, 3, 3.5, 4, 4.5, 5, 5.5, 6, 6.5, 7, 7.5, 8, 8.5, 9, 9.5, 10, 11, 12, 13, 14, 15, or 20 mg/kg. These doses can be administered one or more times (e.g., 2, 3, 4, 5, 6, 7, 8, 9, 10, 11, or 12 or more times) per day, week, month, or year. For example, an sFGFR3 polypeptide can be administered to patients in a weekly dosage ranging, e.g., from about 0.0014 mg/kg/week to about 140 mg/kg/week, e.g., about 0.14 mg/kg/week to about 105 mg/kg/week, or, e.g., about 1.4 mg/kg/week to about 70 mg/kg/week (e.g., 5 mg/kg/week).

### Gene Therapy

An sFGFR3 polypeptide can be administered to a patient as a nucleic acid molecule. Examples of nucleic acid molecules that can be administered include those that encode sFGFR3 polypeptides having the amino acid sequence of any one of SEQ ID NOs: 1-7 or a variant thereof having at least 85% sequence identity thereto (e.g., 86%-100% sequence identity, such as 86%, 87%, 88%, 89%, 90%, 91%, 92%, 93%, 94%, 95%, 96%, 97%, 98%, or 99% sequence identity thereto). For example, the nucleic acid molecules may have the sequence of any one of SEQ ID NOs: 10-18 or a variant thereof with at least 85% sequence identity thereto (e.g., 86%-100% sequence identity, such as 86%, 87%, 88%, 89%, 90%, 91%, 92%, 93%, 94%, 95%, 96%, 97%, 98%, or 99% sequence identity thereto).

The sFGFR3 polypeptide-encoding nucleic acid molecules can be delivered by gene therapy, in which a polynucleotide encoding the sFGFR3 polypeptide is delivered to tissues of interest and expressed in vivo. Gene therapy methods are discussed, e.g., in Verme et al. (Nature 389:239-242, 1997), Yamamoto et al. (Molecular Therapy 17:S67-S68, 2009), and Yamamoto et al., (J. Bone Miner. Res. 26: 135-142, 2011), each of which is hereby incorporated by reference.

An sFGFR3 polypeptide of the invention can be produced by the cells of a patient (e.g., a human) having increased visceral fat deposition by administrating a vector (e.g., a plasmid, an artificial chromosome (e.g. BAG, PAC, and YAC), or a viral vector) containing the nucleic acid sequence of a polynucleotide encoding the sFGFR3 polypeptide. For example, a viral vector can be a retroviral vector, adenoviral vector, or poxviral vector (e.g., vaccinia viral vector, such as Modified Vaccinia Ankara (MVA)), adeno-associated viral vector, or alphaviral vector. The vector, once inside a cell of the patient (e.g., a human) having a skeletal growth retardation disorder (e.g., achondroplasia), by, e.g., transformation, transfection, electroporation, calcium phosphate precipitation, or direct microinjection, will promote expression of the sFGFR3 polypeptide, which is then secreted from the cell. The invention further includes cell-based therapies, in which the patient (e.g., a human) is administered a cell expressing the sFGFR3 polypeptide.

### Pharmaceutical Compositions

Pharmaceutical compositions that can be administered to treat a subject having abnormal visceral fat deposition, as discussed herein, contain an sFGFR3 polypeptide, a polynucleotide encoding the sFGFR3 polypeptide, or a host cell that contains the sFGFR3 polynucleotide. The sFGFR3 polypeptide can have the amino acid sequence of any one of SEQ ID NOs: 1-7 or a variant thereof having at least 85% sequence identity thereto (e.g., 86%-100% sequence identity, such as 86%, 87%, 88%, 89%, 90%, 91%, 92%, 93%, 94%, 95%, 96%, 97%, 98%, or 99% sequence identity thereto). The nucleic acid molecules may have the sequence of any one of SEQ ID NOs: 10-18 or a variant thereof with at least 85% sequence identity thereto (e.g., 86%-100% sequence identity, such as 86%, 87%, 88%, 89%, 90%, 91%, 92%, 93%, 94%, 95%, 96%, 97%, 98%, or 99% sequence identity thereto). Compositions including an sFGFR3 polypeptide, a polynucleotide encoding the sFGFR3 polypeptide, or a host cell that contains the sFGFR3 polynucleotide can be formulated at a range of dosages, in a variety of formulations, and in combination with pharmaceutically acceptable excipients, carriers, or diluents.

A pharmaceutical composition including an sFGFR3 polypeptide, a polynucleotide encoding the sFGFR3 polypeptide, or a host cell that contains the sFGFR3 polynucleotide can be formulated at a specific dosage, such as a dosage that is effective for treating a patient (e.g., a human) with increased visceral fat deposition without inducing significant toxicity. For example, the compositions can be formulated to include between about 1 mg/mL and about 500 mg/mL of the sFGFR3 polypeptide or polynucleotide (e.g., between 10 mg/mL and 300 mg/mL, 20 mg/mL and 120 mg/mL, 40 mg/mL and 200 mg/mL, 30 mg/mL and 150 mg/mL, 40 mg/mL and 100 mg/mL, 50 mg/mL and 80 mg/mL, or 60 mg/mL and 70 mg/mL of the sFGFR3 polypeptide or polynucleotide).

The pharmaceutical compositions including an sFGFR3 polypeptide or polynucleotide can be prepared in a variety of forms, such as a liquid solution, dispersion or suspension, powder, or other ordered structure suitable for stable storage. For example, compositions including an sFGFR3 polypeptide or polynucleotide intended for systemic or local delivery can be in the form of injectable or infusible solutions, such as for parenteral administration (e.g., subcutaneous, intravenous, intramuscular, intra-arterial, intrathecal, or intraperitoneal administration). sFGFR3 compositions for injection (e.g., subcutaneous or intravenous injection) can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (2nd ed.) Taylor & Francis Group, CRC Press (2006), which is hereby incorporated by reference in its entirety.

Compositions including an sFGFR3 polypeptide or polynucleotide can be provided to patients (e.g., humans) having increased visceral fat deposition in combination with pharmaceutically acceptable excipients, carriers, or diluents. Acceptable excipients, carriers, or diluents can include buffers, antioxidants, preservatives, polymers, amino acids, and carbohydrates. Aqueous excipients, carriers, or diluents can include water, water-alcohol solutions, emulsions or suspensions including saline, buffered medical parenteral vehicles including sodium chloride solution, Ringer's dextrose solution, dextrose plus sodium chloride solution, Ringer's solution containing lactose, and fixed oils. Examples of non-aqueous excipients, carriers, or diluents are propylene glycol, polyethylene glycol, vegetable oil, fish oil, and injectable organic esters.

Pharmaceutically acceptable salts can also be included in the sFGFR3 compositions. Exemplary pharmaceutically acceptable salts can include mineral acid salts (e.g., hydrochlorides, hydrobrom ides, phosphates, and sulfates) and salts of organic acids (e.g., acetates, propionates, malonates, and benzoates). Additionally, auxiliary substances, such as wetting or emulsifying agents and pH buffering substances, can be present. A thorough discussion of pharmaceutically acceptable excipients, carriers, and diluents is available in Remington: The Science and Practice of Pharmacy, 22^{nd} Ed., Allen (2012), which is hereby incorporated by reference in its entirety.

Pharmaceutical compositions including an sFGFR3 polypeptide or polynucleotide can also be formulated with a carrier that will protect the sFGFR3 polypeptide or polynucleotide against rapid release, such as a controlled release formulation, including implants and microencapsulated delivery systems. For example, the sFGFR3 composition can be entrapped in microcapsules prepared by coacervation techniques or by interfacial polymerization, such as hydroxymethylcellulose, gelatin, or poly-(methylmethacylate) microcapsules; colloidal drug delivery systems (e.g., liposomes, albumin microspheres, microemulsions, nano-particles, or nanocapsules); or macroemulsions. Additionally, an sFGFR3 composition can be formulated as a sustained-release composition. For example, sustained-release compositions can include semi-permeable matrices of solid hydrophobic polymers containing the sFGFR3 polypeptide or polynucleotide, in which the matrices are in the form of shaped articles, such as films or microcapsules.

### EXAMPLES

The following examples are intended to illustrate, rather than limit, the disclosure.

### Example 1

Study Design

To evaluate the potential of sFGFR3 therapy (e.g., sFGFR3 having the sequence of SEQ ID NO: 1) to prevent the development of abdominal obesity in achondroplasia, we have first characterized the development of obesity in children by doing a retrospective chart review and have then conducted a study in Fgfr3ach/+ mice, a mouse model recapitulating most human symptoms. For the chart review, eleven subjects (5 girls and 6 boys) with achondroplasia were followed from birth up to 18 years in the Department of endocrinology, bone diseases, genetic and medical gynecology of the Purpan Children's Hospital in Toulouse, France. This retrospective chart review study was conducted in accordance with the declaration of Helsinki and the French regulations on Biomedical research, not requiring ethics committee approval for a non-interventional study. All patients harbored the G380R FGFR3 mutation (G380R refers to the numbering in the full length FGFR3, including the signal peptide; the residue numbering without the signal peptide is G358R (see SEQ ID NO: 9), confirmed by molecular testing. Patients receiving any growth treatment were excluded. Children were followed in the specialized center every 4 months on average. During each visit, data included the height, weight and subsequent body mass index (BMI=kg/m2). Blood analyses were performed only once a year and dual-energy X-ray absorptiometry (DXA) was performed once. The mouse study was performed on transgenic Fgfr3ach/+ mice or their wild-type (WT) littermates. For this, litters were treated blindly twice weekly by subcutaneous injections of 2.5 μg/kg of sFGFR3 or vehicle starting at day 3 until day 21. Mice were weaned at age 3 weeks. After one week of acclimation, mice were challenged with normal (ND) or high-fat diet (HFD) for 10 weeks. The development of obesity was evaluated through measures of body composition, indirect calorimetry and classical evaluation of glucose and lipid profiles, as well as hepatic and pancreatic function evaluations. The n per group is presented in the figure legends.

Clinical Analysis

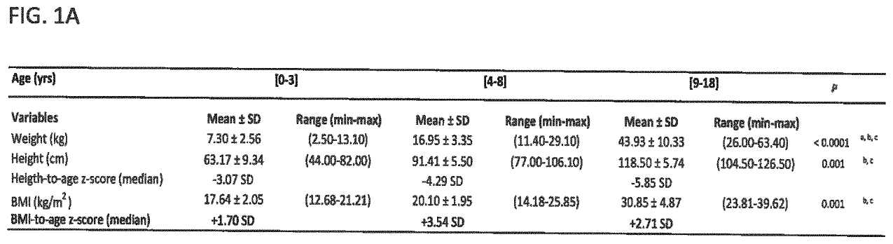

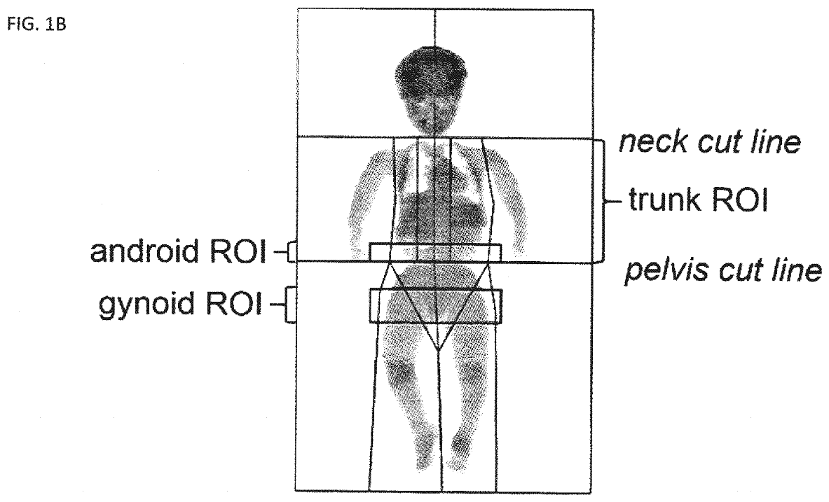

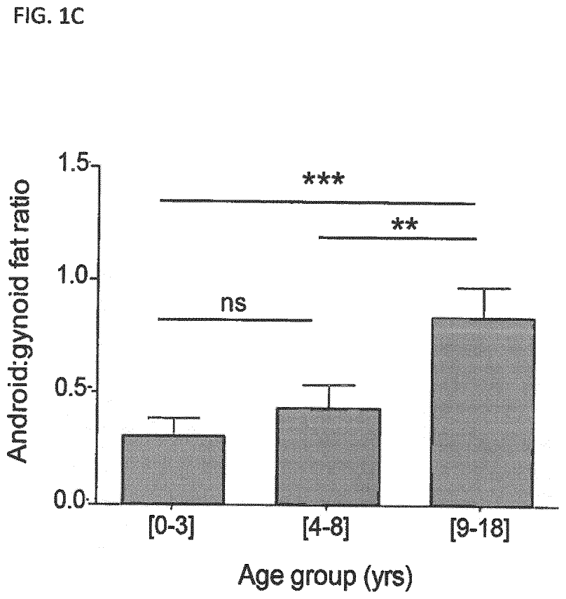

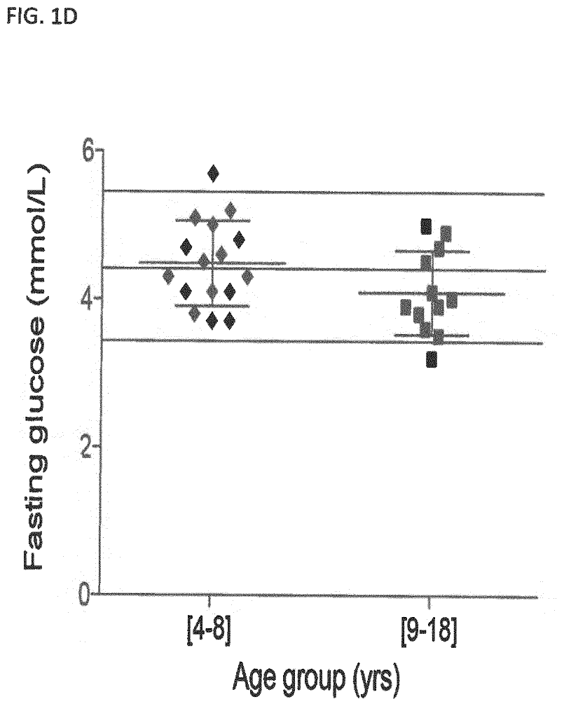

Anthropometric calculations, including height-to-age z score and BMI-to-age z score, were done using the WHO AnthroPlus software (WHO AnthroPlus for personal computers Manual: Software for assessing growth of the world's children and adolescents. Geneva: WHO, 2009 (www.who.int/growthref/tools/en/); z-scores are based on WHO standards (birth to 60 months) and WHO reference 2007 (61 months to 19 years). Body composition was evaluated by DXA using the Lunar Prodigy device (GE Healthcare). The regions of interest (ROI) for regional body composition were defined using the manufacturer's instructions (FIG. 1B). Briefly, the trunk ROI was measured from the pelvis cut (lower boundary) to the neck cut (upper boundary); the android ROI was measured from the pelvis cut (lower boundary) to above the pelvic cut by 20% of the distance between the neck and the pelvis cuts (upper boundary); the umbilicus ROI was defined from the lower boundary of the android by 150% of the android distance; and the gynoid ROI was from the lower boundary of the umbilicus ROI to a line equal to twice the height of the android ROI (lower boundary). Blood samples were drawn after at least a 12-hour fasting period and analyzed at the Federative Institute of Biology (IFB) of the Purpan hospital. Fasting glucose and insulin, total, HDL, and LDL cholesterol, triglyceride, TGO, TGP, gGT concentrations, as well as plasma total calcium, sodium, potassium, bicarbonate, phosphate, chloride and alkaline phosphatase were measured using standard colorimetric or colorimetric enzymatic methods on the Cobas 8000 modular analyzer series, using the C701 module, from Roche Diagnostics. Serum concentration of total 25OH vitamin D was measured by chemiluminescent immunoassay method on the Cobas 8000 modular analyzer series, using the E602 module, from Roche Diagnostics. All values were compared to reference values established for each age group within the Children's hospital and using published references (Mellerio et al., Pediatrics 129: e1020-1029 (2012); Fischer et al., Ann. Clin. Biochem. 49:546-553, 2012; and Haine et al., J. Bone Miner. Res. 30:1369-1376, 2015). After 12 h overnight fast, OGTT was performed in patients per the established recommendations (Alberti et al., Diabet. Med. 15:539-553, 1998). Following oral administration of 1.75 g/kg of glucose, blood samples were drawn at baseline and after 30 and 120 min to measure glucose concentration using hexokinase method. Glucose regulation was assessed according to the American Diabetes Association guidelines: normal glucose regulation was defined as fasting glucose <5.6 mmol/L and 120 min glucose <7.8 mmol/L and impaired fasting glucose as fasting glucose

5.6-6.9 mmol/L, impaired glucose tolerance as 120 min glucose 7.8-11.0 mmol/L.

Animals and Treatment

Experiments were performed on transgenic Fgfr3ach/+ mice (Naski et al., Development 125: 4977-4988, 1998). At weaning, ear biopsies were used to verify mice genotype by PCR of genomic DNA as previously described (Garcia et al., Science Translational Medicine 5:203ra124, 2013).

During the experiment, mice were housed in standard laboratory conditions and were allowed access food and water ad libitum. The study was approved by the local Institutional Ethic Committee for the use of Laboratory Animals (CIEPAL Azur) (approvals # NCE-2012-52 and NCE-2015-225). At day 3, newborn mice were treated with 2.5 mg/kg of FLAG-tagged sFGFR3 as described previously (Garcia et al., Science Translational Medicine 5:203ra124, 2013). Control litters received 10 μl of PBS containing 50% glycerol (vehicle). From age day 3 to day 22, Fgfr3ach/+ mice received 6 subcutaneous injections of sFGFR3 or vehicle. One week after weaning at 4 weeks of age, treated and untreated mice were divided into two groups and challenged for 10 weeks with normal (ND, A03, SAFE) or high fat diet (HFD, 52% kcal as fat, custom made, containing 54% lipids, SAFE), respectively. After 6 hours fasting, blood was taken from the tail vein. Glycemia was measured with a glucometer (Abbot) and serum insulin contents were determined by ELISA (Mercodia). Glucose tolerance tests (GTT) were performed on mice after 10 weeks of ND or HFD challenge. After 6 hours fasting mice were injected with an intra-peritoneal glucose solution (1 g/kg). Blood was taken from the tail vein and glucose levels were monitored over time using a glucometer or using EnzyChrom Glucose Assay Kit (BioAssay Systems). Glucose levels were normalized to the value of time −15 min of each mouse.

Indirect calorimetry was studied on mice challenged for 10 weeks with normal (ND) or high fat (HFD) diet. As stated above, mice were treated during the growth period with 2.5 mg/kg of sFGFR3 or vehicle, were subjected to the diet challenge for 2 weeks and were then subjected to the metabolic chambers. After 24 h of acclimatization in individual metabolic cages, 02 consumption (VO2) and CO2 production (VCO2) were measured (Oxylet; Panlab-Bioseb) in individual mice at 32 min intervals during a 24 h period with unrestricted access on food followed by one night fasted. The respiratory quotient was calculated and analyzed as follows: RQ=VCO2/VO2, RQ=1 correspond to carbohydrate oxidation and RQ˜0.7 correspond to fat oxydation. Energy expenditure (kcal/day/weigh×0.75=1.44×VO2×[3.815+1.232×RQ]), carbohydrate (g/min/kg0.75=[4.55×VCO2]-[3.21×VO2]) and lipid (g/min/kg0.75=[1.67×VO2]-[1.67×VCO2]) oxidations were calculated. Ambulatory activities and rearing of the mice were monitored by a weight transducer technology or an infrared photocell beam interruption method (Oxylet; Panlab-Bioseb).

Body composition was determined using a SkyScan 1178 X-ray micro-CT system. Four and 10 weeks old mice were anesthetized and scanned using the same parameters: 104 μm of pixel size, 49 kV, 0.5 mm thick aluminum filter, 0.9° of rotation step. Total adipose tissue volume was determined between the tip of the snout and the top of the tail and abdominal adipose tissue volume was determined between the lumbar L1 and the sacral S1. Then, adipose tissue quantification was carried out more precisely. Body compositionanalysis is based on the delimitation of region of interest after 3D reconstruction of scanned images. 3-D reconstructions analyses were performed using NRecon and CTAn software (Skyscan).

At sacrifice, animals were weighted and several tissues and organs (subcutaneous, epididymal adipose tissue, liver, pancreas) were harvested for further analysis by histochemistry or qPCR. In some groups, bone-marrow-derived mesenchymal stem cells were harvested by flushing the femurs (Zhu et al., Nat Protoc 5:550-560, 2010).

Lipid profile was evaluated by taking intracardiac blood. Total cholesterol, triglycerides (TG), High Density Lipoprotein (HDL) and Low Density Lipoprotein (LDL) were measured on serum using a Beckman AU 2700 Analyzer. Sera were analyzed for protein levels of selected adipokines related to inflammation, obesity, insulin pathway or FGFs using the Mouse Adipokine Array (# ARY013, R&D Systems) according to the manufacturer's instructions on nitrocellulose membranes. Following streptavidin-HRP and chemiluminescent detection the proteins bound to each captured antibody were quantified using densitometry and levels were compared to percent change from WT mice.

Mesenchymal Stem Cell Studies

Mesenchymal stem cells were isolated from femoral bone marrow providing from untreated or sFGFR3-treated 6 to 8 weeks old mice by flushing the femurs (Zhu et al., Nat. Protoc. 5:550-560, 2010) and cultivated to 80% confluence in medium supplemented with 10% serum. Then, medium was replaced with adipogenic medium (DMEM F12 supplemented with 2% serum, 1% antiobiotics, 66 mM insulin, 1 nM triiodothyronine, 100 mM cortisol, 10 μg/ml transferrin and 3 μM rosiglitazone in DMEM-F12). Total RNAs were extracted using Trizol Reagent (Life Technologies) and Chloroform (Sigma). One microgram of total RNA was reverse transcribed into cDNA using random hexamers and the Superscript II Reverse Transcriptase kit (Invitrogen). Real-time qPCR was performed on a StepOne Plus Real-Time PCR System (Applied Biosystems) with Fast SYBR Green Master Mix (Sigma) using a custom RT2 Profiler PCR Array (CAPM13080). Studied genes are listed in Table 1. Gene expression was normalized to HPRT, RPL13a and RPL6 housekeeper gene. FGFRs expression was performed using the following primers: FGFR1, For-5′ CAGATGCACTCCCATCCTCG 3′ Rev-5′ TCT GGGGATGTCCAGTAGGG 3′; FGFR2 For-5′ TGGCAGTGAAGATGTTGAAAG 3′ Rev-5′ ATCATCTTCATCATCTCCATCTCTTG 3′; FGFR3 For-5′ TTATCCTTGGCTCCTGGGTG 3′ Rev-5′ CTGGAAGGTAGCAGTGGGAA 3′; FGFR4 For-5′ GCTCGGAGGTAGAGGTCTTGT 3′ Rev-5′ CCACGCTGACTGGTAGGAA 3′; HSP90 For-5′ TTTGGTGGACACAGGCATTG 3′ Rev-5′ CAAACTGCCCGATCATGGAG 3′.

TABLE 1
List of genes studied using custome RT2 Profiler PCR Array in mesenchymal stem cells.

| Gene Symbol | Official Full Name | Refseq # |
|---|---|---|
| Adipoq | Adiponectin, C1Q and collagen domain containing | NM_009605 |
| Cebpa | CCAAT/enhancer binding protein (C/EBP), alpha | NM_007678 |
| Cebpb | CCAAT/enhancer binding protein (C/EBP), beta | NM_009883 |
| Cebpd | CCAAT/enhancer binding protein (C/EBP), delta | NM_007679 |
| Dlk1 | Delta-like 1 homolog (Drosophila) | NM_010052 |
| Fabp4 | Fatty acid binding protein 4, adipocyte | NM_024406 |
| Fasn | Fatty acid synthase | NM_007988 |
| Gata3 | GATA binding protein 3 | NM_008091 |
| Hprt | Hypoxanthine guanine phosphoribosyl transferase | NM_013556 |
| Lep | Leptin | NM_008493 |
| Lipe | Lipase, hormone sensitive | NM_010719 |
| Lpl | Lipoprotein lipase | NM_008509 |
| Pparg | Peroxisome proliferator activated receptor gamma | NM_011146 |
| Ppargc1a | Peroxisome proliferative activated receptor, gamma, coactivator 1 alpha | NM_008904 |
| Rpl13a | Ribosomal protein L13a | NM_009438 |
| Rpl6 | Ribosomal protein L6 | NM_011290 |
| Scd1 | Stearoyl-Coenzyme A desaturase 1 | NM_009127 |
| Slc2a4 | Solute carrier family 2 (facilitated glucose transporter), member 4 | NM_009204 |
| Srebf1 | Sterol regulatory element binding transcription factor 1 | NM_011480 |
| Ucp1 | Uncoupling protein 1 (mitochondrial, proton carrier) | NM_009463 |
| Wnt1 | Wingless-related MMTV integration site 1 | NM_021279 |

For insulin signaling experiments, 24 h after isolation, cells were depleted during 6 h and then stimulated with 50 nM insulin for 0, 5, 15, 30 min, or with 0, 1, 10, 50, 100 nM for 5 min. Cells extracts were processed for Western Blot analysis. For this, cells were lysed in RIPA lysis buffer (Millipore) and homogenized from 30 min using a vortex. Proteins were pelleted by centrifugation 1400 g from 15 min and total protein contents were evaluated using the BCA Protein Assay Kit (ThermoScientific). Samples were diluted in Sample Reducing Buffer (Life Technology), boiled, and processed for immunoblotting by using a standard procedure. Monoclonal antibodies were used as follows: anti p44/42 MAPK (Erk1/2) (4695S, Cell Signaling), anti phospho-p44/42 MAPK (Erk1/2) (Thr202/Tyr204) (4370S, Cell Signaling). Results were normalized to HSP90 expression (4877S, Cell Signaling).

Histology and Immunohistochemistry

Histology analyses were performed on adipose tissue, liver and pancreas of 12 weeks old mice. Organs were fixed in 4% formalin for 24 h, paraffin-embedded and 5 μm sections are stained with hematoxylin and eosin. Adipocyte diameters were measured in one or two different sections in each sample (from 100 to 300 adipocytes were counted in each section).

Pancreatic islet numbers and area were measured in one section using Fiji Image J system (islet area was normalized to total pancreas area). Liver glycogen content was evaluated by Periodic acid-Shill (PAS) staining. Immunohistochemistry was performed on 5 μm sections of pancreas. Sections were blocked 45 min with PBS 1% BSA, incubated over night with anti-insulin monoclonal primary antibody (4 μg/ml) (Santa Cruz, sc-9168), anti-glucagon polyclonal antibody (4 μg/ml) (Santa Cruz, sc-7779) and 1 h with Alexa Fluor 594 secondary antibody (2 μg/ml) (Life Technologies, A-21442) and Alexa Fluor 488 secondary antibody (4 μg/ml) (Life Technologies, A-21467) in wet chamber. Sections were counterstained with DAPI solution (Santa Cruz), treated with autofluorescence eliminator reagent and visualized under fluorescence microscopy. Staining without secondary antibody was used as a negative control.

Statistical Analysis

Statistical analyses were performed with GraphPad Prism 6.0 software. Data were expressed as mean±SD. To verify normality and equal variance, an Agostino and Pearson omnibus normality test (a=0.05), a Shapiro-Wilk normality test (a=0.05) and a KS normality test (a=0.05) were performed. Significance was determined by using unpaired two-tailed Student t test or Tukey's multiple comparison test. In patients, the raw results of height and BMI were transformed to age-specific z-score from the average in the reference population using reference data. The expected mean result of these values in a healthy population is 0. P values <0.05 were considered significant (* P<0.05; ** P<0.01; *** P<0.001).

### Example 2

Achondroplasia Patients Develop an Excess of Abdominal Adipose Tissue without Classical Complications

Subjects were children and adolescents with achondroplasia. They were included in a longitudinal, retrospective study conducted by the same observer for an average of 8.6±5.6 years. Anthropometric measures and body composition were recorded from birth on during follow up visits and compared between three age groups ranging from [0-3], [4-8] and [9-18] years old. Several metabolic blood parameters were measured and compared in the different age groups. Blood values for visits under age of 3 were not considered because of the difficulty to restrict food intake in infants and thus control metabolic parameters at this young age.

It is known that achondroplasia patients not only display impaired growth but that they also have a tendency to gain excessive weight leading to overweight or even obesity (Hoover-Fong et al., Am. J. Med. Genet. A. 143A:2227-2235, 2007). As seen in FIG. 1A, the BMI of patients with achondroplasia significantly increased during childhood, to reach a value of about 30 kg/m2 in the [9-18] years old group (P=0.2407 between [0-3] and [4-8] groups, P<0.0001 comparing the [9-18] group to both other groups, Tukey's multiple comparisons test). A negative correlation was observed between BMI and height (Pearson r coefficient=−0.5660, P=0.0021), with a tendency for the smallest children to have the highest BMI. To study their metabolic status and its evolution, densitometry analyses were first performed to determine body fat and lean mass distribution in the different age groups (FIG. 1B). We observed that up to 8 years old, the total fat:lean ratio was relatively constant and that it rose significantly during adolescence, from 0.21±0.02 and 0.20±0.05 in the [0-3] and [4-8] age groups to 0.84±0.29 in the [9-18] age group, respectively (P=0.9954 between [0-3] and [4-8] groups, P=0.0053 and P<0.0001 comparing [9-18] to [0-3] and [4-8] age groups, respectively, Tukey's multiple comparisons test) (Table 2).

TABLE 2
Densitometry results of achondroplasia patients in the three age groups. Values are mean ± SD (range). The p values
represent the significance of the difference between the three groups (one way ANOVA test followed by Tukey's multiple
comparison tests, ns = non-significant). Results of post hoc analyses: a significantly different between [0-3]
and [4-8] groups; b significantly different between [0-3] and [9-18] groups; c significantly different between [4-8] and [9-18] groups.

| | Age (yrs) | | | | | | | |
|---|---|---|---|---|---|---|---|---|
| | [0-3] | | [4-8] | | [9-18] | | | |
| | Variables | | | | | | | |
| | Mean ± SD | Range (min-max) | Mean ± SD | Range (min-max) | Mean ± SD | Range (min-max) | p | |
| Fat and lean mass | | | | | | | | |
| repartition (kg) | | | | | | | | |
| Total body fat mass | 1.52 ± 0.32 | (1.29-1.76) | 2.64 ± 0.73 | (1.98-4.06) | 19.78 ± 7.98 | (8.56-30.15) | <0.0001 | ^{b,c} |
| Total body lean mass | 0.88 ± 0.04 | (0.86-0.91) | 0.84 ± 0.09 | (0.75-0.90) | 0.46 ± 0.01 | (0.46-0.68) | 0.001 | ^{b,c} |
| Trunk mass | 3.67 ± 0.45 | (3.38-4.02) | 6.93 ± 1.46 | (4.83-8.68) | 21.19 ± 5.53 | (14.08-28.72) | <0.0001 | ^{b,c} |
| Trunk fat mass | 0.41 ± 0.12 | (0.32-0.49) | 0.83 ± 0.44 | (0.23-1.64) | 9.74 ± 4.42 | (3.97-15.65) | 0.001 | ^{b,c} |
| Trunk lean mass | 3.29 ± 0.33 | (3.06-3.53) | 6.11 ± 1.15 | (4.35-7.08) | 11.4 ± 1.50 | (9.67-13.42) | <0.0001 | ^{a,b,c} |
| Leg mass | 1.77 ± 0.28 | (1.57-1.97) | 4.61 ± 1.29 | (3.12-7.21) | 13.89 ± 3.36 | (8.86-18.24) | <0.0001 | ^{b,c} |
| Leg fat mass | 0.65 ± 0.12 | (0.57-0.73) | 1.22 ± 0.31 | (0.86-1.69) | 7.46 ± 2.61 | (3.39-10.72) | <0.0001 | ^{b,c} |
| Leg lean mass | 1.11 ± 0.16 | (0.99-1.23) | 3.39 ± 1.04 | (2.26-5.52) | 6.31 ± 0.98 | (4.81-7.51) | <0.0001 | ^{a,b,c} |
| Arm mass | 0.65 ± 0.04 | (0.62-0.68) | 1.21 ± 0.23 | (0.99-1.53) | 3.74 ± 1.03 | (2.38-5.12) | <0.0001 | ^{b,c} |
| Arm fat mass | 0.09 ± 0.06 | (0.08-0.10) | 0.21 ± 0.09 | (0.12-0.35) | 1.57 ± 0.76 | (0.56-2.54) | 0.004 | ^{b,c} |
| Arm lean mass | 0.56 ± 0.04 | (0.53-0.58) | 0.99 ± 0.18 | (0.66-1.18) | 2.16 ± 0.34 | (1.62-2.65) | 0.001 | ^{b,c} |
| Fat and lean mass | | | | | | | | |
| repartition (% of | | | | | | | | |
| total body weight) | | | | | | | | |
| Total body fat mass | 19.04 ± 0.74 | (18.51-19.57) | 16.08 ± 3.10 | (11.86-21.40) | 42.93 ± 9.11 | (28.36-52.89) | <0.0001 | ^{b,c} |
| Total body lean mass | 88.29 ± 3.51 | (85.81-90.77) | 83.89 ± 9.25 | (74.75-90.43) | 46.19 ± 0.79 | (45.63-68.49) | <0.0001 | ^{b,c} |
| Trunk mass | 46.47 ± 2.57 | (44.64-48.29) | 47.26 ± 4.08 | (28.42-50.15) | 50.35 ± 0.06 | (45.00-50.40) | ns | |
| Trunk fat mass | 5.01 ± 0.64 | (4.56-5.46) | 5.60 ± 1.74 | (1.37-8.65) | 27.11 ± 0.50 | (13.16-27.46) | <0.0001 | ^{b,c} |
| Trunk lean mass | 41.47 ± 3.22 | (39.19-43.74) | 41.66 ± 5.82 | (27.05-45.78) | 23.24 ± 0.44 | (22.71-33.47) | 0.004 | ^{b,c} |
| Leg mass | 22.14 ± 0.37 | (21.88-22.40) | 25.22 ± 0.28 | (25.03-42.45) | 31.13 ± 1.24 | (29.68-32.32) | 0.0285 | ^{c} |
| Leg fat mass | 8.17 ± 0.02 | (8.16-8.19) | 6.94 ± 0.87 | (5.95-9.95) | 18.05 ± 1.08 | (11.23-19.32) | <0.0001 | ^{b,c} |
| Leg lean mass | 13.97 ± 0.40 | (13.69-14.26) | 18.28 ± 0.60 | (17.64-32.50) | 13.07 ± 0.16 | (12.96-18.44) | 0.0122 | ^{c} |
| Arm mass | 8.21 ± 0.88 | (7.59-8.83) | 7.33 ± 0.21 | (6.66-8.07) | 8.89 ± 0.14 | (7.60-8.99) | 0.0032 | ^{c} |
| Arm fat mass | <0.001 | (10⁻⁶-10⁻⁵) | 1.08 ± 0.42 | (0.78-1.88) | 4.40 ± 0.09 | (1.86-4.46) | <0.0001 | ^{b,c} |
| Arm lean mass | 7.05 ± 0.76 | (6.51-7.59) | 6.25 ± 0.21 | (5.44 ± 6.60) | 4.49 ± 0.23 | (4.25-6.04) | 0.0005 | ^{b,c} |
| Gynoid and android body | | | | | | | | |
| composition (% total tissue) | | | | | | | | |
| Gynoid body fat | 43.75 ± 0.07 | (43.70-43.80) | 33.97 ± 3.00 | (29.30-38.40) | 52.91 ± 6.37 | (40.60-58.80) | 0.0007 | ^{c} |
| Android body fat | 13.45 ± 3.47 | (11.00-15.90) | 14.73 ± 4.02 | (8.90-19.50) | 44.79 ± 11.39 | (29.90-55.90) | <0.0001 | ^{b,c} |
| Gynoid body lean | 56.25 ± 0.07 | (56.20-56.30) | 67.52 ± 4.70 | (60.90-74.91) | 47.10 ± 6.38 | (40.50-58.70) | ns | |
| Android body lean | 86.6 ± 3.40 | (84.20-89.00) | 85.27 ± 3.97 | (80.52-91.07) | 55.21 ± 11.39 | (44.10-69.20) | ns | |

Interestingly, as seen in FIG. 1C, the increase in fat mass was not homogeneous and patients preferentially developed abdominal (android) fat mass (+204%) over hip (gynoid) fat mass (+55%) (P=0.0974 between [0-3] and [4-8] groups, P=0.0002 and P=0.001 comparing [9-18] to [0-3] and [4-8] age groups, respectively, Tukey's multiple comparisons test). Concurrently, both android and gynoid lean masses did not vary during this period (Table 1). Consequently, the fat:lean ratio significantly increased in the abdominal area throughout childhood (P=0.3187 between [0-3] and [4-8] groups, P=0.0924 and P<0.0001 comparing [9-18] to [0-3] and [4-8] age groups, respectively, Tukey's multiple comparisons test). The trunk, legs and arms followed a very similar trend with an increase in percent fat mass and decrease in percent lean mass from infancy to adulthood (Table 1). Spinal bone mineral density (BMD) was determined between L1 and L4 after age of 3. In both age groups, BMD was found to be below age-appropriate normal range value (van der Sluis et al., Arch. Dis. Child. 87:341-347, 2002) (0.511±0.065 g/cm2 and 0.898±0.223 g/cm² in the [4-8] and [9-18] age groups, respectively, compared to 0.645±0.071 g/cm2 and 0.913±0.199 g/cm2 in the same age referenced groups).

Different blood parameters were compared between the [4-8] and [9-18] age groups. Unexpectedly, in both age groups and independently of their BMI, achondroplasia children displayed a tendency to low plasmatic total cholesterol (3.38±0.36 mmol/L and 3.73±0.44 mmol/L in the [4-8] and [9-18] age groups, respectively, with normal values being comprised between 3.90 and 5.70 mmol/L in children), and low triglycerides (0.56±0.14 mmol/L and 0.63±0.13 mmol/L in the [4-8] and [9-18] age groups, respectively, with normal values being comprised between 0.60 and 1.70 mmol/L in children). Similarly, fasting blood glucose (FIG. 1D) and insulin levels were not increasing with age and remained within normal range (7.3±5.4 mUl/L and 13.4±3.4 mUl/L in the [4-8] and [9-18] age groups, respectively, with normal values being comprised between 2.6 and 16 mUl/L in children). These results were confirmed by glucose levels obtained during oral glucose tolerance test (OGTT), that showed normal glucose levels at 0, 30 and 120 minutes following oral administration (4.53±0.22 mmol/l at 0 min, 7.97±1.72 mmol/l at 30 min and 5.17 mmol/l at 120 min). No statistical differences were found between both age groups. Due to high levels of hemolysis during the OGTT, no data were unfortunately available regarding insulin levels. All other blood parameters were within normal range (data not shown).

Metabolic Alterations are Observed in Lean and Obese Fgfr3ach/+ Mice and are Corrected by sFGFR3 Treatment

To determine the role of FGFR3ach in this preferential development of visceral obesity in achondroplasia, transgenic Fgfr3ach/+ mice carrying the G380R mutation or their wild-type (WT) littermates were treated with sFGFR3 or vehicle for 3 weeks starting at day 3. Mice were then challenged with normal (ND) or high fat diet (HFD) starting at 4 weeks of age for a duration of 10 weeks to evaluate the development of obesity.

After weaning, at 4 weeks of age, as expected, untreated Fgfr3ach/+ mice had a 20.4% decrease in body weight compared to their WT littermates. This was associated with reduced lean and fat tissues (50% and 33.9% respectively). Treated animals displayed a 14.1% decrease in body weight compared to WT mice (P<0.0001).

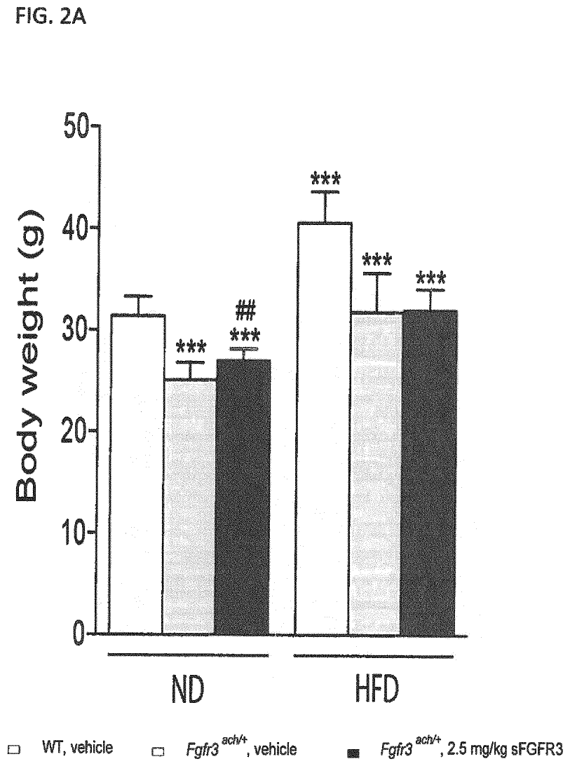

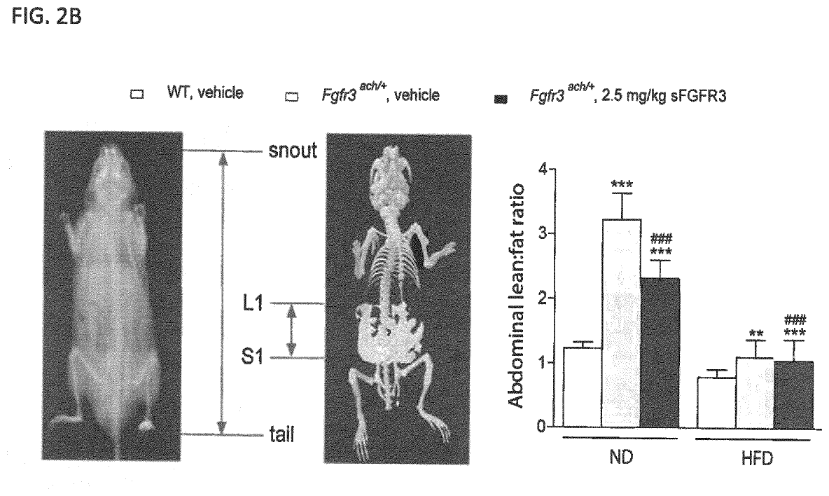

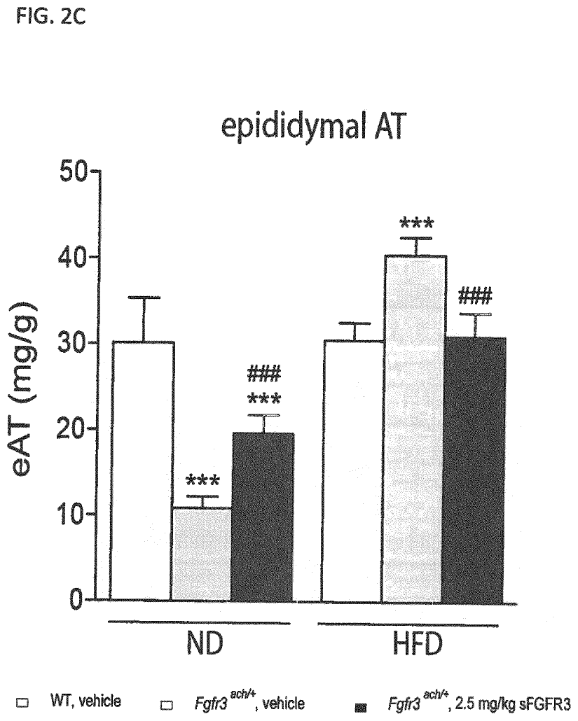

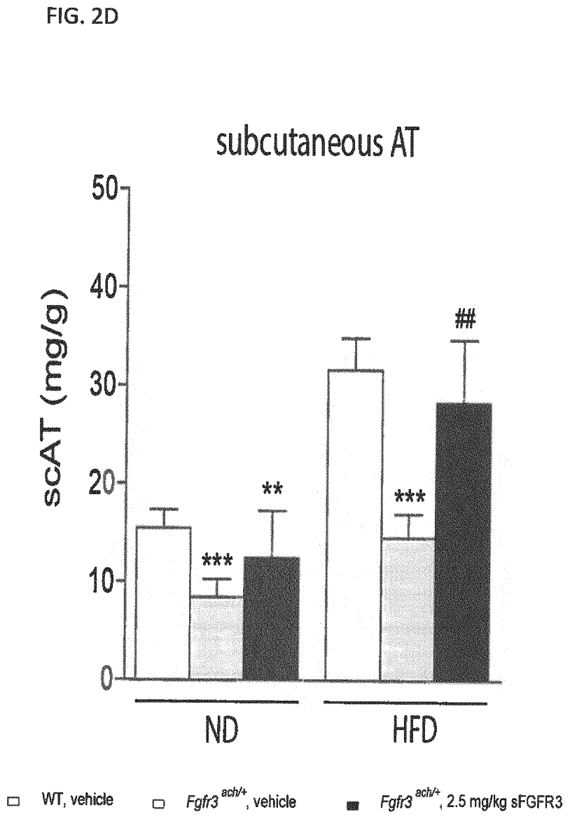

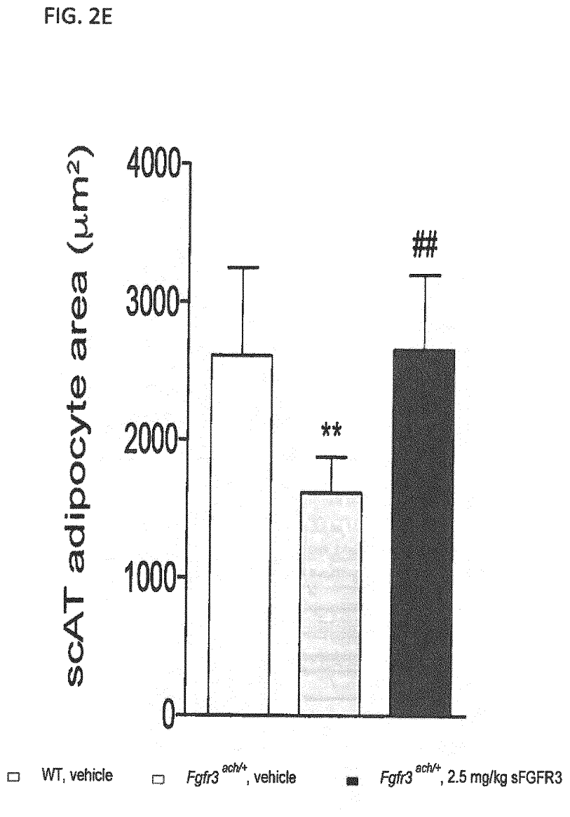

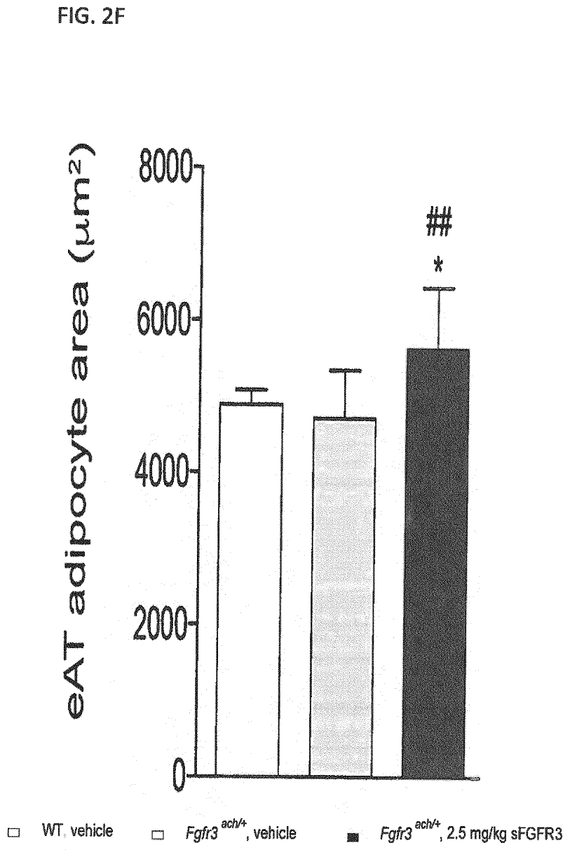

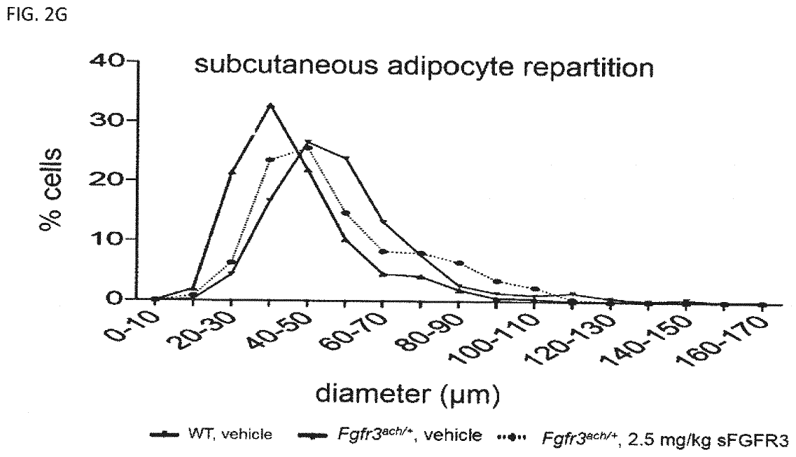

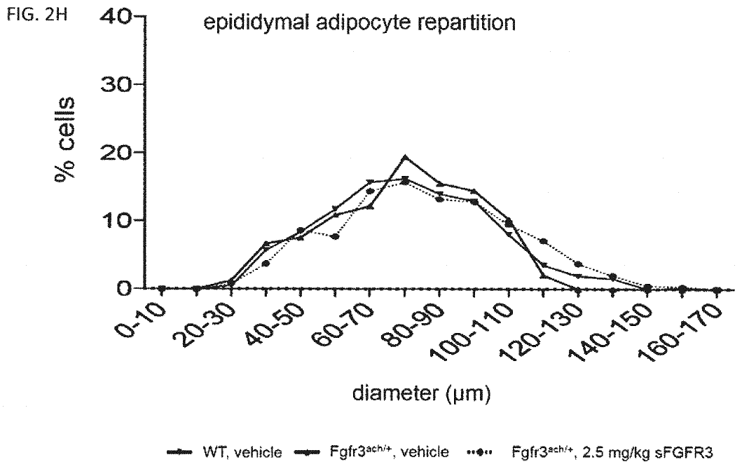

After 10 weeks of diet challenge, all HFD groups showed a significant increase in body weight compared to ND (FIG. 2A). Interestingly however body composition was different in both genotypes. Untreated Fgfr3ach/+ mice had an higher abdominal lean:fat ratio, measured between L1 and S1, compared to WT mice, independently of the diet (FIG. 2B). When fed with ND, untreated Fgfr3ach/+ mice had less epididymal (visceral) and subcutaneous adipose tissues than WT animals (FIGS. 2C and 2D). However, after 10 weeks of HFD challenge, untreated Fgfr3ach/+ mice developed more epididymal adipose tissue compared to WT animals that preferentially developed subcutaneous fat depot (FIG. 2C and 2D). These data showed that, like achondroplasia patients, Fgfr3ach/+ mice were prone to developed visceral adipose tissue.

sFGFR3 treatment has no effect on body weight gain (FIG. 2A). However, body composition was significantly impacted by sFGFR3 treatment with a significant decrease in abdominal lean:fat ratio in mice fed with ND (FIG. 2B), caused by a decrease in lean masses and an increase in fat masses respectively. Very interestingly, sFGFR3-treated Fgfr3ach/+ mice displayed fat depot distributions that were like those of WT animals whether they were fed with ND or HFD (FIGS. 2C and 2D).

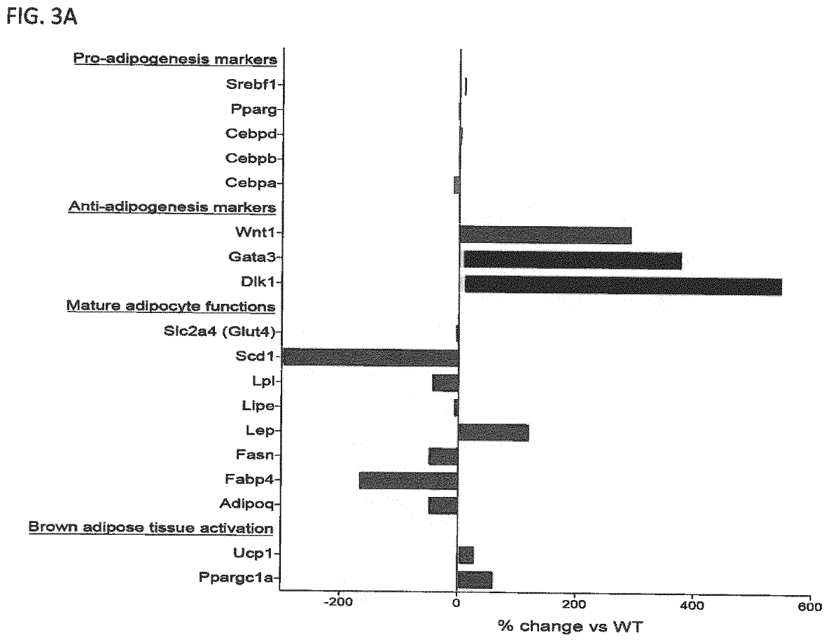

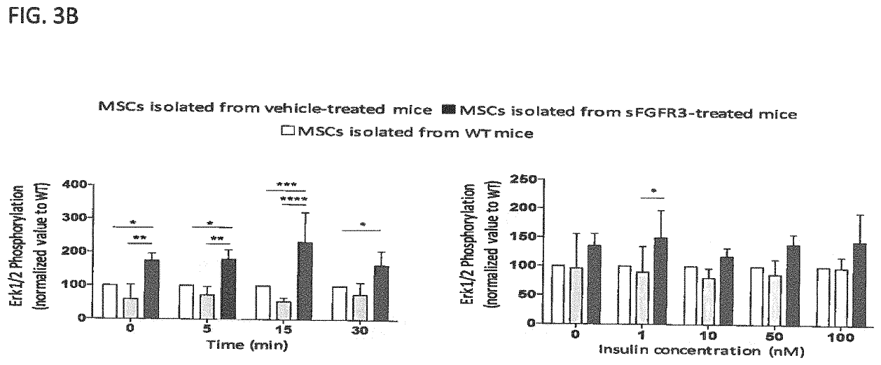

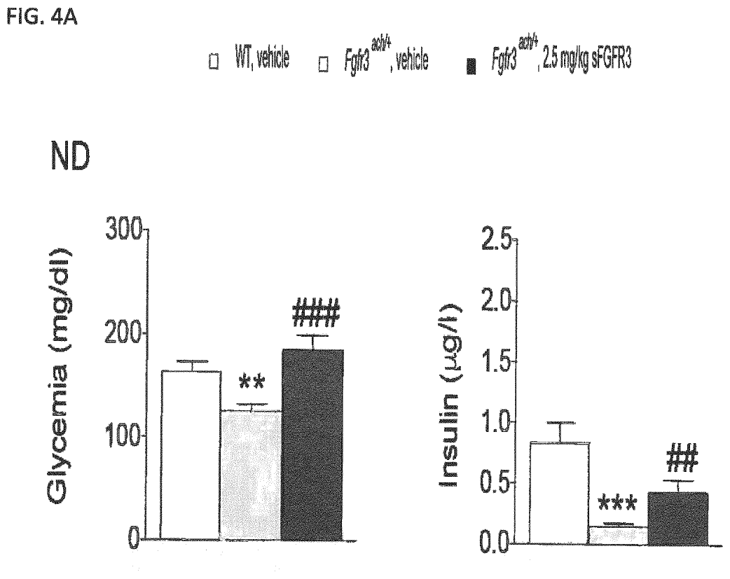

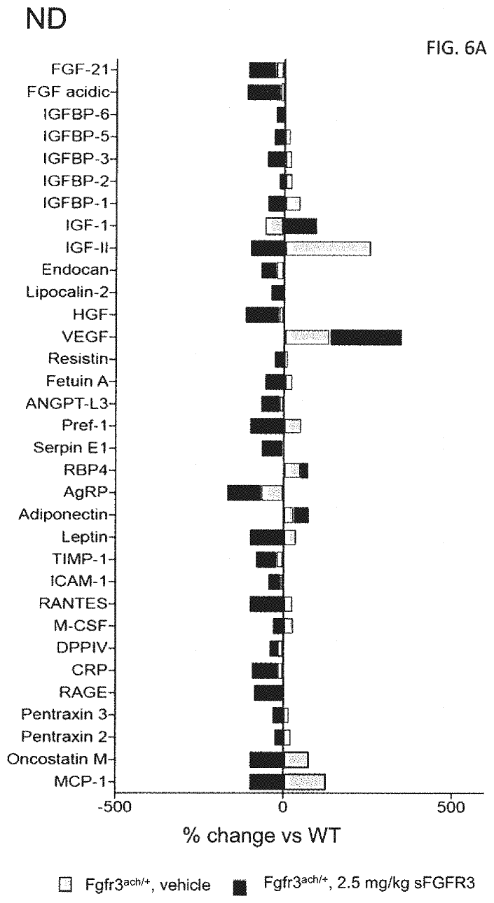

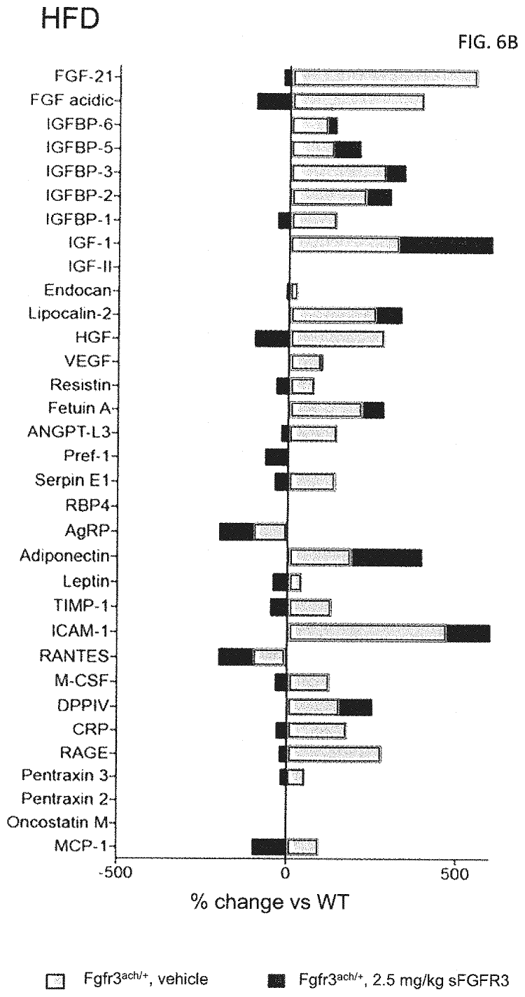

After the HFD challenge, histological analyses showed smaller adipocytes in the subcutaneous area and a greater proportion of these small adipocytes in untreated Fgfr3ach/+ mice compared to WT (FIGS. 2E and 2G). No difference in size or dispersion was observed in epididymal adipocytes between both genotypes (FIGS. 2F and 2H). sFGFR3 treatment restored the subcutaneous adipocytes size and scattering and induced a slight increase in epididymal adipocyte size (FIG. 2E-2H). Because increased proportion of small adipose cells has been associated with inflammation (Kursawe et al., Diabetes 59: 2288-2296, 2010; and Lafontan, Diabetes Metab. 40:16-28, 2014), several circulating adipokines were measured to evaluate the extent of systemic inflammation in these animals (FIGS. 6A and 6B). Adipokines were sorted into four categories—pro-inflammatory, obesity-related, insulin-pathway and FGFs—all of which were increased in transgenic mice compared to WT littermates (Table 3). Untreated Fgfr3ach/+ mice displayed a low-grade inflammatory baseline compared to WT animals, that was exacerbated under HFD challenge (Table 3). Treated Fgfr3ach/+ animals under ND or HFD has a systemic profile that resembled that of their WT littermates. In vitro, mesenchymal stem cells (MSCs) isolated from Fgfr3ach/+ mice showed that early and intermediary genes of the differentiation process such as Srebf-1, CEBP/d, CEPB/a and PPARg were already expressed (FIG. 3A). Very interestingly, MSCs isolated from sFGFR3-treated Fgfr3ach/+ mice showed significant increase in anti-adipogenic markers and brow tissue activation markers as well as decreased expression of genes involved in the functions of mature adipocytes (FIG. 3A). Together with the in vivo data, this suggests a predisposition to adipogenesis in Fgfr3ach/+ mice that can be prevented by sFGFR3 treatment.

TABLE 3
Untreated Fgfr3^{ach/+} mice displayed an elevated inflammatory baseline prevented
by sFGFR3 treatment. Pro-inflammatory, obesity, insulin pathway and FGF circulating
adipokines expression performed into vehicle-treated WT and Fgfr3^{ach/+} mice and
sFGFR3 treated Fgfr3^{ach/+} after 10 weeks of ND or HFD challenge.

| | ND | | | HFD | | |
|---|---|---|---|---|---|---|
| | | Fgfr3^{ach/+} | | | Fgfr3^{ach/+} | |
| | WT | | 2.5 mg/kg | WT | | 2.5 mg/kg |
| markers (n) | vehicle | vehicle | sFGFR3 | vehicle | vehicle | sFGFR3 |
| Pro-inflammatory (14) | + | ++ | + | + | ++ | + |
| Obesity (15) | ++ | +++ | + | ++ | +++ | ++ |
| Insulin pathway (7) | ++ | ++ | ++ | ++ | +++ | ++ |
| FGF (2) | + | + | − | − | ++ | − |

‘−’ = <2 arbitrary units (A.U.),
‘+’ = 10-30 A.U.,
‘++’ = 30-100 A.U.,
‘+++’ >100 A.U.

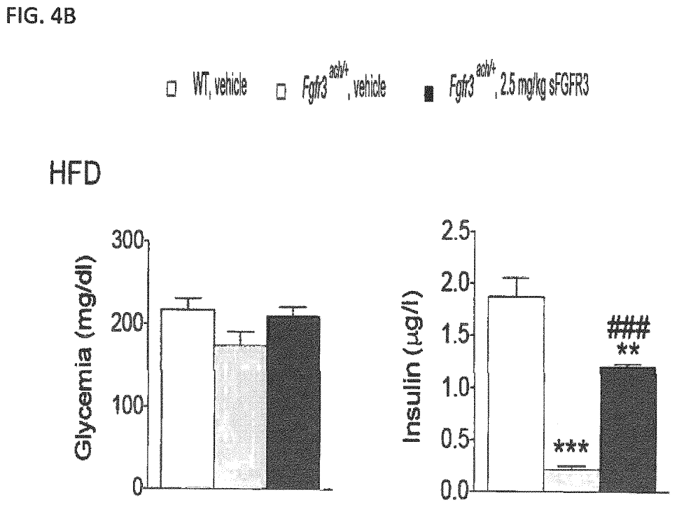

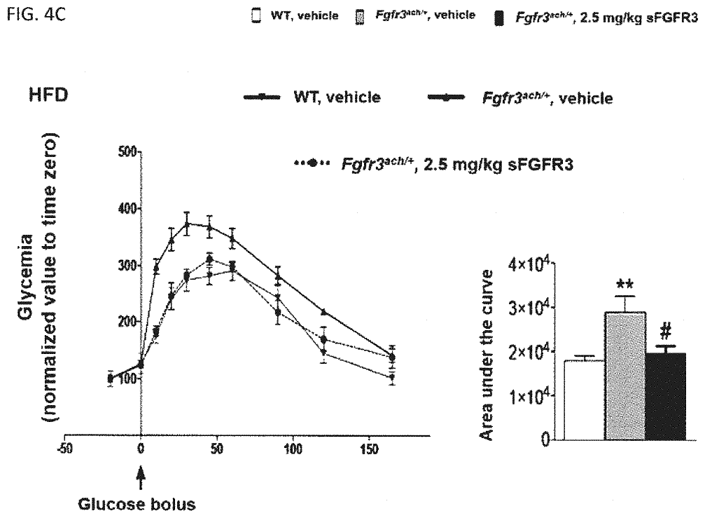

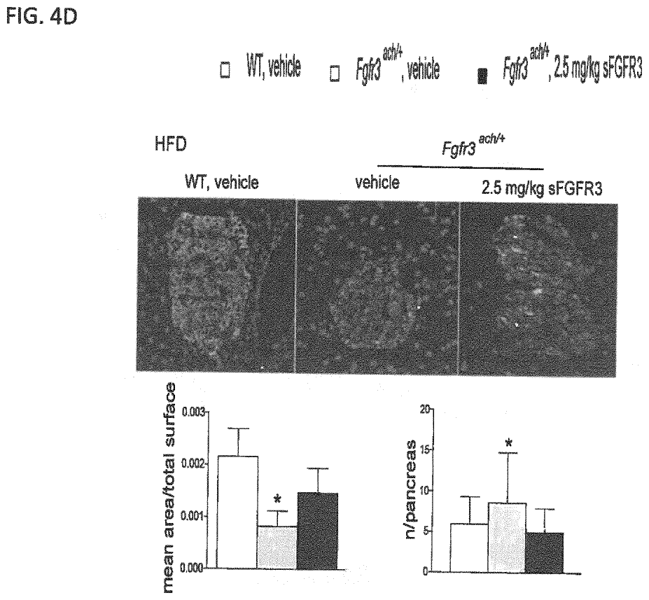

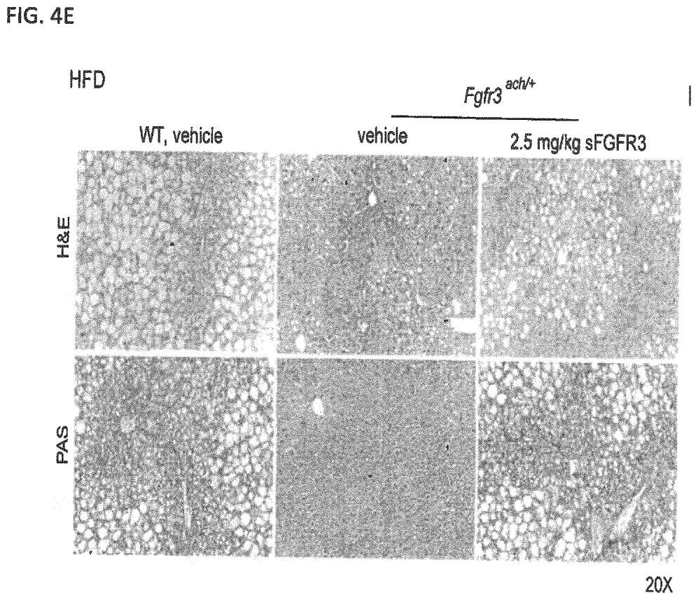

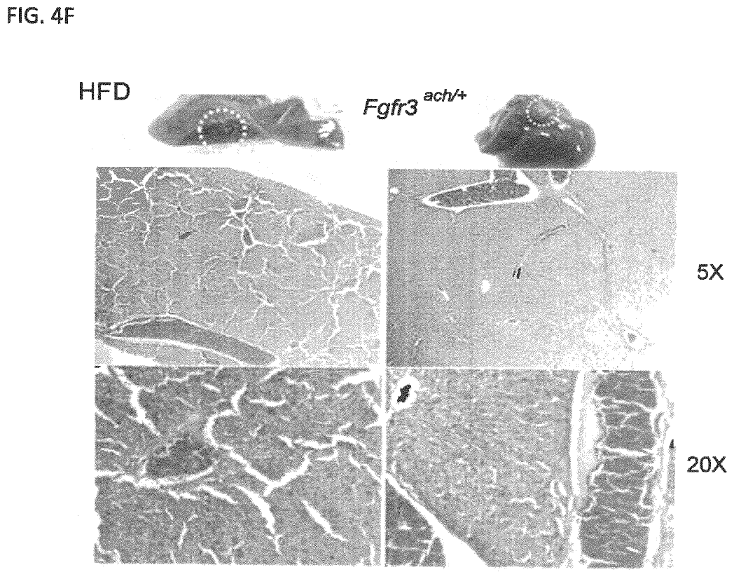

Compared to their WT littermates, mice carrying the FGFR3 mutation had low fasting glycemia and very low baseline levels of insulin (FIG. 4A). When challenged with a HFD diet (FIG. 4B), glycemic levels raised but remained under those of the WT animals. Insulin levels remained extremely low. In sFGFR3-treated Fgfr3ach/+ animals, glycemia was restored and insulin levels significantly increased compared to untreated Fgfr3ach/+ mice (FIGS. 4A and 4B).

To evaluate the development of glucose intolerance, glucose tolerance tests (GTT) were performed after 10 weeks of diet challenge. Untreated Fgfr3ach/+ mice displayed higher glucose levels and AUC compared to their WT littermates (Cmax 320.9±32.0 mg/dL and 273.3±23.9 mg/dL, AUC 1.2×104±0.7×104 and 1.7×104±0.4×104, respectively), showing some basal glucose intolerance even under ND. This was further exacerbated under HFD (FIG. 4C). When Fgfr3ach/+ mice were treated with 2.5 mg/kg sFGFR3, normal GTT responses were restored (FIG. 4C). We attempted to perform insulin tolerance test in transgenic mice under ND or HFD but, because of their lower basal glycemic levels, Fgfr3ach/+ mice did not support insulin injection and died rapidly. Analysis of insulin sensitivity of mesenchymal stem cells isolated from Fgfr3ach/+ or WT mice showed no differences in Erk1/2 phosphorylation levels suggesting similar response to insulin stimulation in both type of mice (FIG. 3B). These results suggest that Fgfr3ach/+ mice do not appear more sensitive to insulin regulation but that insulin injection during the ITT probably induced lethal hypoglycemia because of their low basal glycemia. Pancreas analyses showed smaller and more islets of Langerhans with lower insulin and glucagon contents in untreated Fgfr3ach/+ mice (FIG. 4D) suggesting an alteration of insulin production and/or storage. This was partially restored in treated animals (FIGS. 3B and 4A-4C). Glucose storage also appeared impaired in the liver of untreated Fgfr3ach/+ animals as seen by the decrease in glycogen in liver sections (FIG. 4E). As expected, following 10 weeks of HFD challenge, WT mice developed grade III macrovesicular steatosis with more than 75% of hepatocytes displayed lipid vacuoles that were larger than the nucleus (FIG. 4E). In contrast, after 10 weeks of HFD, untreated Fgfr3ach/+ mice developed reversible benign hepatic nodules (FIG. 4F) and a grade II microvesicular steatosis: less than 50% of hepatocytes displayed small vacuoles (FIG. 4E). Interestingly, sFGFR3 treatment restored a normal hepatic response in treated Fgfr3ach/+ mice (FIG. 4E) and no nodules were observed.

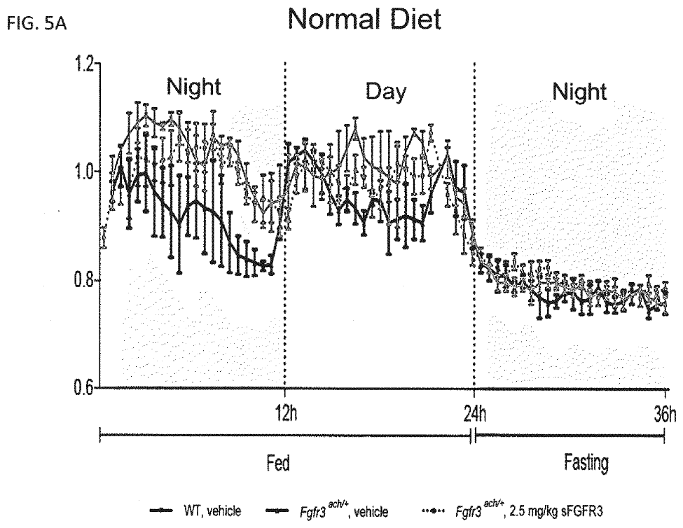

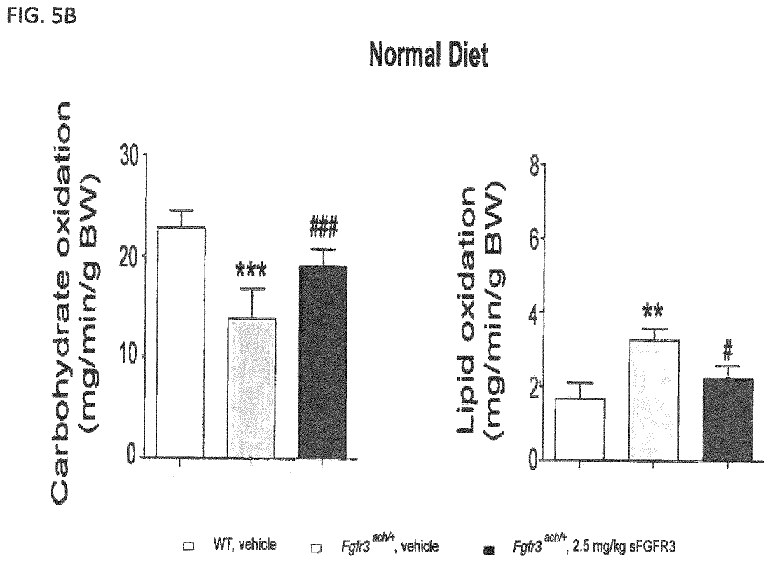

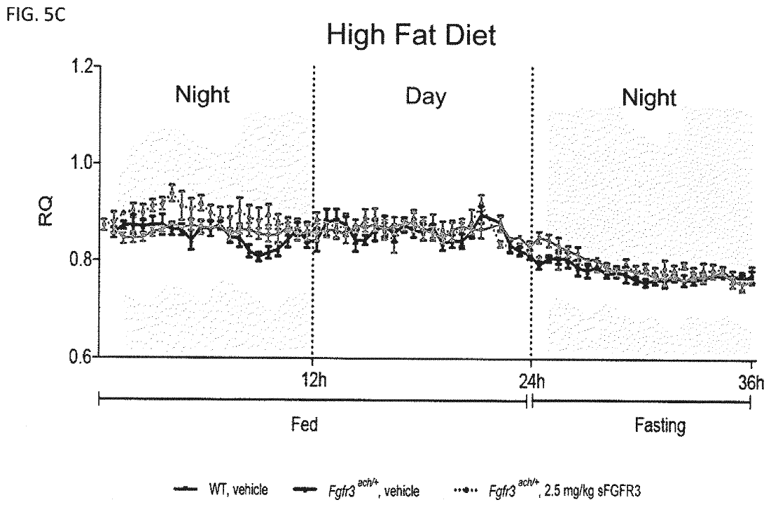

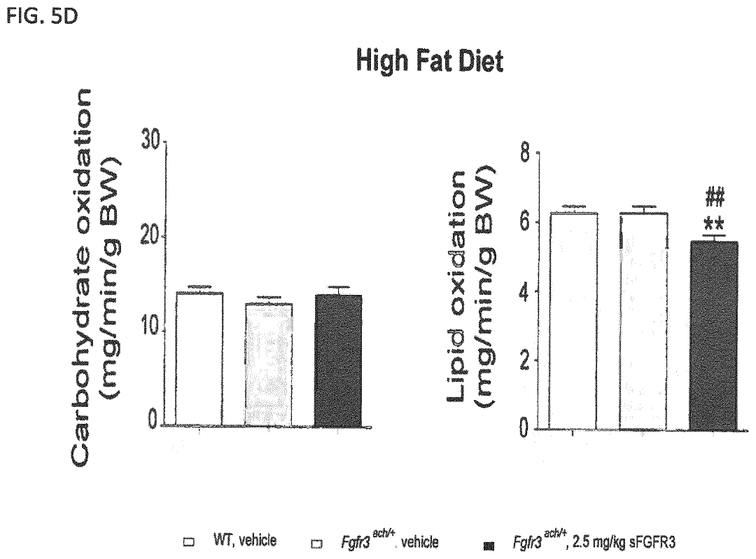

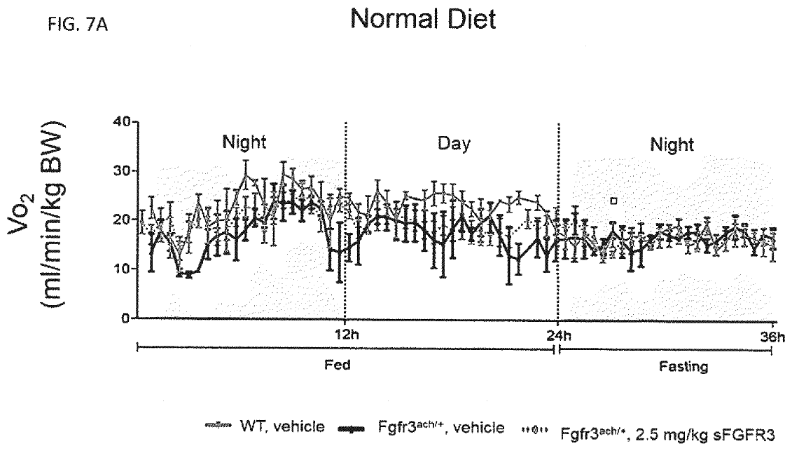

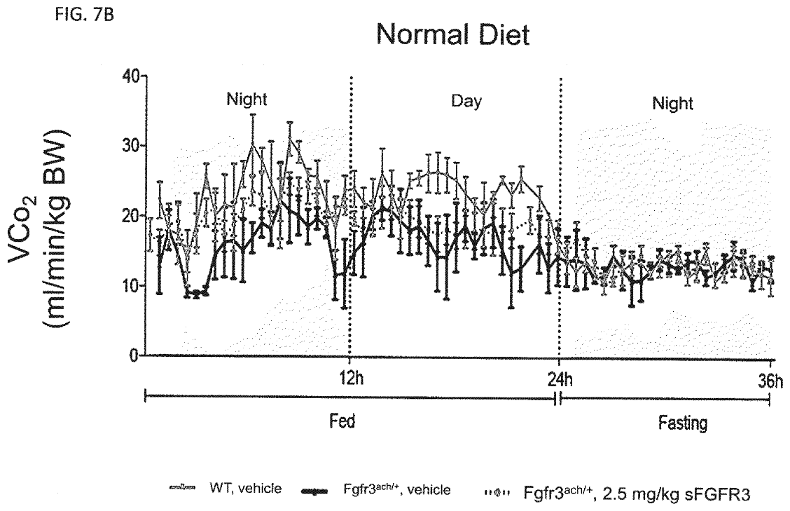

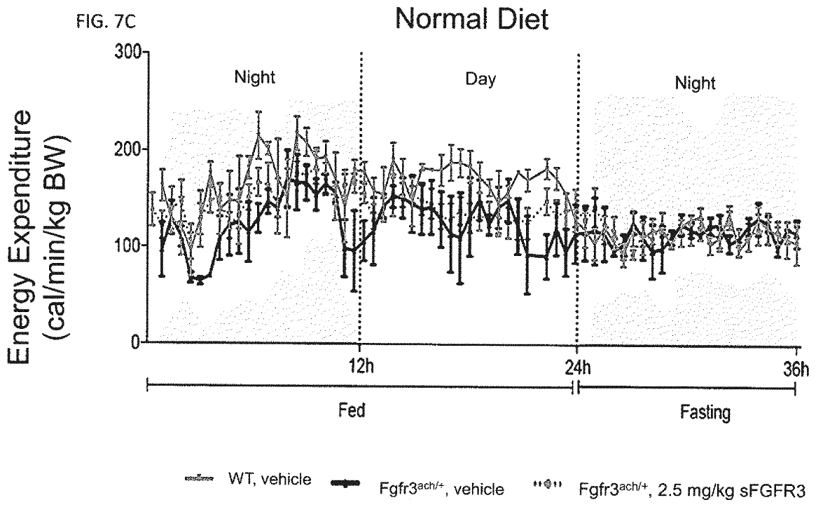

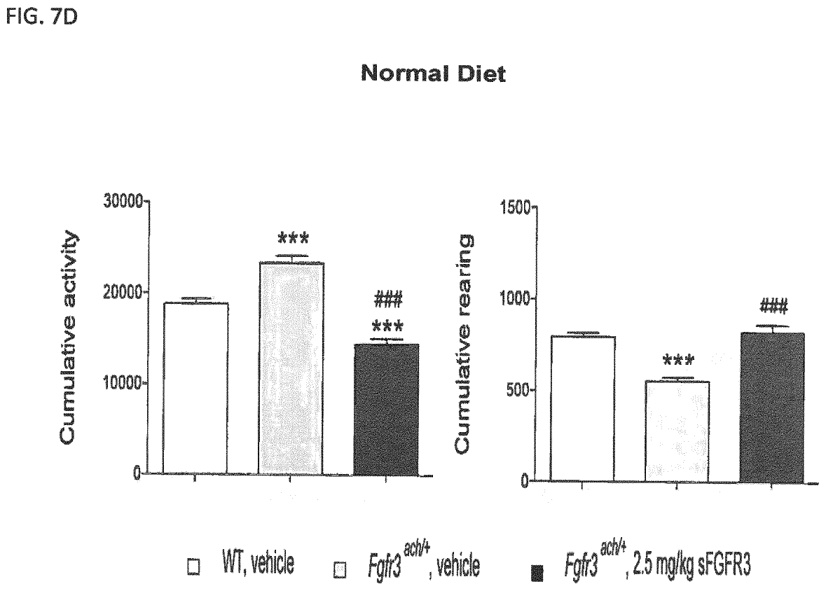

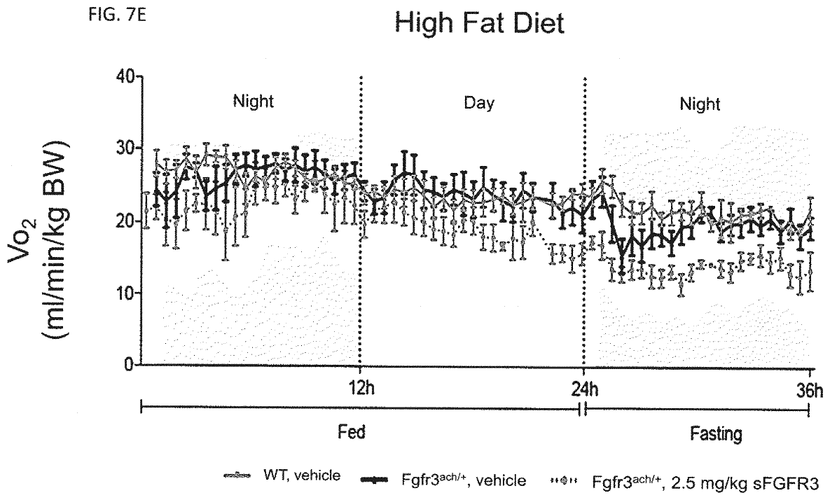

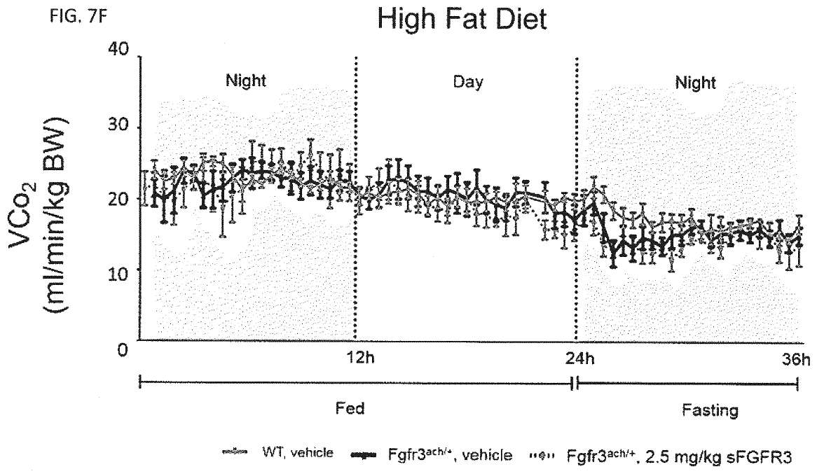

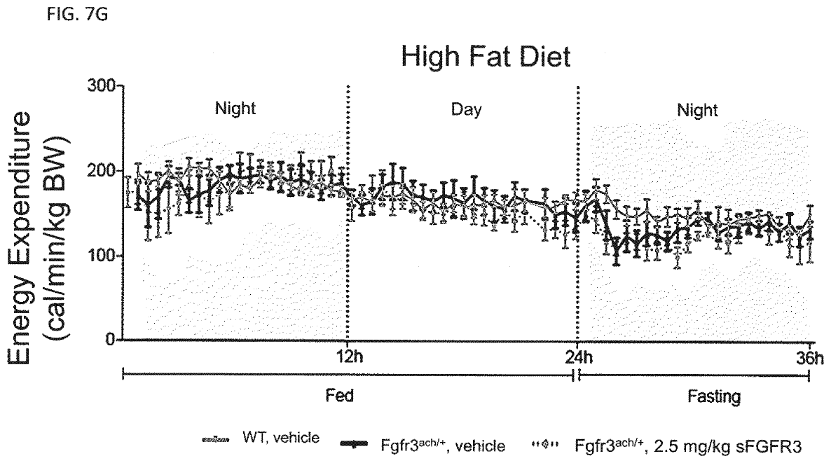

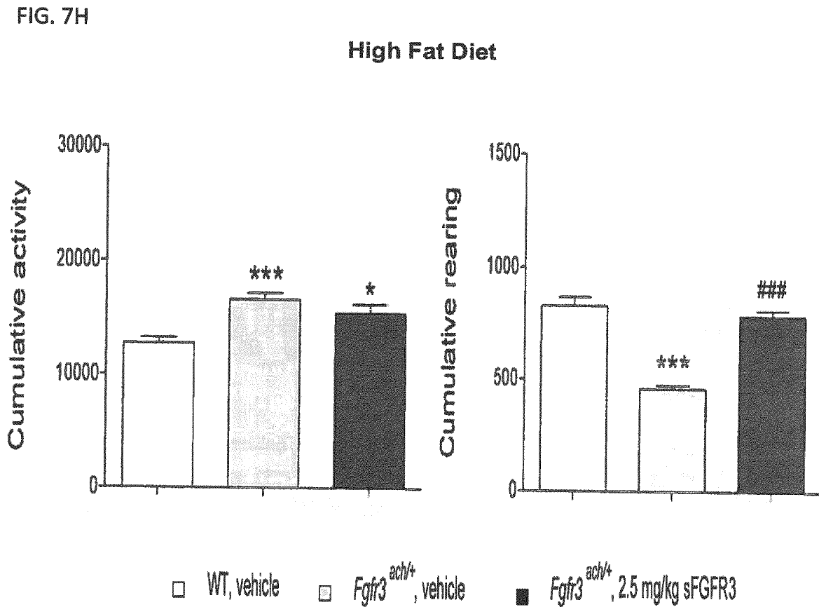

The basal energy metabolic rate of Fgfr3ach/+ mice was evaluated by indirect calorimetry. We found that while lean WT animals fed with normal diet (ND) drew energy from carbohydrate sources (respiratory quotient RQ near 1), fed transgenic achondroplasia mice drew their energy essentially from lipid sources (RQ near 0.7) (FIG. 5A). In fasting episode, as expected, both types of animals drew their energy from lipid sources. This preferential lipid utilization was confirmed by the calculation of carbohydrate and lipid oxidation, which were respectively lower and higher in Fgfr3ach/+ mice compared to WT animals (FIG. 5B). Over a 24-hour period, Fgfr3ach/+ mice tend to eat constantly not only during the nocturnal period, however energy expenditure and food intake were not significantly different between both genotypes (FIGS. 7A-D). As expected under HFD, all animals drew their energy from lipid sources, leading to similar carbohydrate and lipid oxidation indexes (FIGS. 5C-D, FIGS. 7E-H). Very interestingly, Fgfr3ach/+ animals that received sFGFR3 treatment during the growth period behaved like untreated WT mice after weaning whether they were fed with ND or HFD, suggesting the restoration of glucose metabolism capacities during the treatment period (FIG. 5 and FIGS. 7A-H).

Variations in adipose tissue deposition in the different body sites can be assessed to evaluate the severity of obesity in achondroplasia. Compared to BMI z-scores and skinfold measurements, the android:gynoid ratio is closely related to all risk factors in overweight and obese children. In achondroplasia, children display higher android:gynoid ratio that develops very early during childhood. Our findings in Fgfr3ach/+ mice suggests a predisposition to preferential visceral obesity in these patients. Indeed, cells derived from the mesenchymal lineage appeared more prone to adipogenesis than cells isolated from WT animals and seem pre-engaged in the differentiation process towards adipocytes. Moreover, different adipocyte distribution was observed with a greater proportion of small adipocytes in the subcutaneous adipose tissue of Fgfr3ach/+ mice compared to WT animals. The number of fat cells is set during childhood and adolescence and remains constant during adulthood. Obesity in achondroplasia patients may be set very early during childhood and monitoring as early as 4-8 years old using, e.g., DXA scans, may be warranted.

The development of an abdominal obesity is usually considered to be the most deleterious type of obesity (Smith, J. Clin. Invest. 125:1790-1792, 2015). Our results suggest that abdominal obesity is likely a consequence of the mutation affecting FGFR3. Currently, three members of the FGFs family have been linked to obesity (Nies et al., Front. Endocrinol. (Lausanne) 6:193, 2015): FGF1, FGF15/19 and FGF21. FGF1 is regulated by PPARg and is notably highly upregulated in WAT (28). FGF1 is known to promote pre-adipocyte proliferation and differentiation through Erk1/2 signaling. It also triggers acute blood lowering effect that seem to be dependent on FGFR2 signaling in WAT. FGF15/19 is considered as a regulator of the feeding responses. It binds to FGFR4/bklotho receptor complex on the cell membrane of hepatocytes ultimately leading to repression of gluconeogenesis (Tomlinson et al., Endocrinology 143:1741-1747, 2002). FGF21 mainly binds to FGFR1 and regulates the adaptive fasting response through PPARa (Kharitonenkov et al., J. Clin. Invest. 115:1627-1635, 2005). In Fgfr3ach/+ mice, overexpression of FGFR3ach during embryogenesis could modify FGFs signaling in different cell types. In accordance with this, we found that mesenchymal stem cells from Fgfr3ach/+ mice express high levels of FGFR3 compared to their WT littermates. Similarly, newborn Fgfr3ach/+ mice express increased levels of FGFR2 and FGFR4 in AT and liver. Altogether these data could explain the low glycemia associated with abdominal obesity in Fgfr3ach/+ mice. Along this line, we also observed that patients even tended to lower fasting glycemia and insulinemia and no patients had glucose intolerance, suggesting that similar mechanisms could apply in human.

sFGFR3 treatment was applied immediately after birth and prevented most metabolic complications, including the development of abdominal obesity. Treated Fgfr3ach/+ mice were not protected against obesity per se but behave essentially like WT animals with the development of homogeneous obesity and the restoration of glucose metabolism leading to glucose resistance under HFD. This suggests that, if apply early in life, treatment could revert the effect of the Fgfr3ach mutation on these atypical metabolic tissues.

In conclusion, our data establish that the development of a nonconforming obesity is preferentially abdominal and appears to be triggered by the FGFR3 mutation. Our data also indicate that the treatment for achondroplastic patients having abnormal visceral fat deposition may be applied to other patient populations with abnormal visceral fat deposition and conditions stemming therefrom. Patients having skeletal growth retardation disorders may be treated to control abnormal visceral fat deposition and conditions associated therewith, for example, after the conclusion of bone growth when sFGFR3 polypeptides would not otherwise be considered a relevant therapy. Patients may also benefit from monitoring for co-morbities of obesity, such as diabetes and CVS risks.

### OTHER EMBODIMENTS

All publications, patents, and patent applications mentioned in the above specification are hereby incorporated by reference to the same extent as if each individual publication, patent, or patent application was specifically and individually indicated to be incorporated by reference in its entirety.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

### SPECIFIC EMBODIMENTS

The following specific embodiments also describe the present invention:

1. A method of treating or reducing abnormal fat deposition in a subject in need thereof comprising administering a soluble fibroblast growth factor receptor 3 (sFGFR3) polypeptide, a polynucleotide encoding the sFGFR3 polypeptide, or a host cell comprising the polynucleotide to the subject.

2. The method of 1, wherein the abnormal fat deposition comprises visceral fat deposition.

3. The method of 2, wherein:

a) the abnormal visceral fat deposition is associated with or surrounding one or more of the following organs: the heart, liver, spleen, kidneys, pancreas, intestines, reproductive organs, and gall bladder;

b) the abnormal visceral fat deposition causes disease in one or more of the following organs: the heart, lungs, trachea, liver, pancreas, brain, reproductive organs, arteries, and gall bladder; or

c) the abnormal visceral fat deposition is caused by dysfunction in an endocrine organ, such as an adrenal gland, a pituitary gland, or a reproductive organ, such as an ovary.

4. The method of any one of 1 to 3, wherein the method reduces or eliminates one or more conditions associated with the abnormal fat distribution.

5. The method of 4, wherein the one or more conditions are selected from the group consisting of obstructive sleep apnea, pulmonary disease, cardiovascular disease, metabolic disease, neurological disease, dyslipidemia, hypertension, atherosclerosis, myocardial infarction, stroke, dementia, infertility, menstrual irregularities, insulin dysregulation, and glucose dysregulation.

6. The method of 5, wherein the dyslipidemia comprises an abnormal level of one or more of triglycerides, high-density lipoproteins (HDLs), low-density lipoproteins (LDLs), and cholesterol.

8. The method of 5, wherein the cardiovascular disease is heart disease or stroke.

9. The method of 5, wherein the pulmonary disease is asthma and restrictive lung disease.

10. The method of 5, wherein the neurological disease is dementia or Alzheimer's disease.

11. The method of 5, wherein the metabolic disease is type 2 diabetes, glucose intolerance, nonalcoholic fatty liver disease and liver toxicity.

12. The method of 5, wherein the insulin dysregulation is insulin resistance.

13. The method of any one of 1 to 12, wherein the subject is not overweight or lacks substantial subcutaneous fat deposition.

14. The method of any one of 1 to 13, wherein the abnormal fat deposition is determined using an anthropometric techniques, or imaging.

15. The method of 14, wherein the anthropometric technique is body mass index (BMI) or android:gynoid fat ratio.

16. The method of 14, wherein the imaging comprises computed tomography (CT), magnetic resonance imaging (MRI), and dual energy x-ray absorptioometry (DXA).

18. The method of any one of 1 to 18, wherein the patient does not exhibit substantial abnormal fat deposition outside the abdomen.

19. The method of any one of 1 to 18, wherein the subject is a fetus, a neonate, an infant, a child, a juvenile, an adolescent, or an adult.

20. The method of any one of 1 to 19, wherein the method reduces visceral fat deposition.

21. The method of any one of 1 to 20, wherein the sFGFR3 polypeptide comprises at least 50 consecutive amino acids of an extracellular domain of a naturally occurring fibroblast growth factor receptor 3 (FGFR3) polypeptide.

22. The method of 21, wherein the sFGFR3 polypeptide comprises 100-370 consecutive amino acids of an extracellular domain of the naturally occurring fibroblast growth factor receptor 3 (FGFR3) polypeptide.

23. The method of 21 or 22, wherein the sFGFR3 polypeptide comprises fewer than 350 amino acids of the extracellular domain of the naturally occurring FGFR3 polypeptide.

24. The method of any one of 21 to 23, wherein the sFGFR3 polypeptide comprises an Ig-like 02-type domain 1, 2, and/or 3 of the naturally occurring FGFR3 polypeptide.

25. The method of any one of 1 to 24, wherein the sFGFR3 polypeptide lacks a signal peptide and/or a transmembrane domain, such as the signal peptide and/or transmembrane domain of a naturally occurring FGFR3 polypeptide.

26. The method of any one of 1 to 25, wherein the sFGFR3 polypeptide is a mature polypeptide.

27. The method of any one of 1 to 26, wherein the sFGFR3 polypeptide comprises 400 consecutive amino acids or fewer of an intracellular domain of a naturally-occurring FGFR3 polypeptide.

28. The method of 27, wherein the sFGFR3 polypeptide comprises between 5 and 399 consecutive amino acids of the intracellular domain of a naturally-occurring FGFR3 polypeptide, such as 175, 150, 125, 100, 75, 50, 40, 30, 20, 15, or fewer consecutive amino acids of the intracellular domain of a naturally-occurring FGFR3 polypeptide.

29. The method of 28, wherein the sFGFR3 polypeptide comprises an amino acid sequence having at least 90%, 92%, 95%, 97%, or 99% sequence identity to amino acids 401 to 413 of SEQ ID NO: 8.

30. The method of 29, wherein the sFGFR3 polypeptide comprises amino acids 401 to 413 of SEQ ID NO: 8.

31. The method of any one of 1 to 30, wherein the sFGFR3 polypeptide lacks a tyrosine kinase domain of a naturally-occurring FGFR3 polypeptide.

32. The method of any one of 1 to 31, wherein the sFGFR3 polypeptide lacks an intracellular domain of a naturally-occurring FGFR3 polypeptide.

33. The method of any one of 1 to 33, wherein the sFGFR3 polypeptide comprises fewer than 475, 450, 425, 400, 375, 350, 300, 250, 200, 150, or 100 amino acids in length.

34. The method of any one of 1 to 33, wherein the sFGFR3 polypeptide comprises an amino acid sequence having at least 85% sequence identity to amino acids residues 1 to 280 of SEQ ID NO: 8.

35. The method of 34, wherein the amino acid sequence of the sFGFR3 polypeptide has 86%-100% sequence identity to amino acids residues 1 to 280 of SEQ ID NO: 8.

36. The method of any one of 1 to 35, wherein the sFGFR3 polypeptide comprises an amino acid sequence having at least 85% sequence identity to the sequence of any one of SEQ ID NOs: 1-7.

37. The method of 36, wherein the amino acid sequence of the sFGFR3 polypeptide has 86%-100% sequence identity to the sequence of any one of SEQ ID NOs: 1-7.

38. The method of any one of 1 to 37, wherein the subject has a skeletal growth retardation disorder, obesity, polycystic ovary syndrome, or hypercortisolism, such as Cushing's disease.

39. The method of 38, wherein the skeletal growth retardation disorder is a FGFR3-related skeletal disease.

40. The method of 39, wherein the FGFR3-related skeletal disease is selected from the group consisting of achondroplasia, thanatophoric dysplasia type I (TDI), thanatophoric dysplasia type II (TDII), severe achondroplasia with developmental delay and Acanthosis nigricans (SADDEN), hypochondroplasia, a craniosynostosis syndrome, and camptodactyly, tall stature, and hearing loss syndrome (CATSHL).

41. The method of 40, wherein the skeletal growth retardation disorder is achondroplasia.

42. The method of 40, wherein the craniosynostosis syndrome is selected from the group consisting of Muenke syndrome, Crouzon syndrome, and Crouzonoderrnoskeletal syndrome.

43. The method of any one of 38 to 42, wherein the FGFR3-related skeletal disease is caused by expression in the patient of a FGFR3 variant that exhibits ligand-dependent overactivation.

44. The method of 43, wherein the FGFR3 variant comprises an amino acid substitution of a glycine residue with an arginine residue at position 358 (G358R) as set forth in SEQ ID NO: 9.

45. The method of any one of 38 to 44, wherein the subject has been diagnosed with the skeletal growth retardation disorder, obesity, polycystic ovary syndrome, or hypercortisolism, such as Cushing's disease.

46. The method of any one of 38 to 45, wherein the subject exhibits one or more symptoms of the skeletal growth retardation disorder selected from the group consisting of short limbs, short trunk, bowlegs, a waddling gait, skull malformations, cloverleaf skull, craniosynostosis, wormian bones, anomalies of the hands, anomalies of the feet, hitchhiker thumb, and chest anomalies.

47. The method of any one of 1 to 37, wherein the subject does not have a skeletal growth retardation disorder, such as a FGFR3-related skeletal disease.

48. The method of 47, wherein the subject does not have an FGFR3-related skeletal disease is selected from the group consisting of achondroplasia, thanatophoric dysplasia type I (TDI), thanatophoric dysplasia type II (TDII), severe achondroplasia with developmental delay and Acanthosis nigricans (SADDEN), hypochondroplasia, a craniosynostosis syndrome, and camptodactyly, tall stature, and hearing loss syndrome (CATSHL).

49. The method of any one of 21 to 25 and 27-32, wherein the naturally-occurring human FGFR3 polypeptide comprises the amino acid sequence of Genbank Accession No. NP_000133.

50. The method of any one of 1 to 49, wherein the sFGFR3 polypeptide binds to a fibroblast growth factor (FGF).

51. The method of 50, wherein the FGF is selected from the group consisting of fibroblast growth factor 1 (FGF1), fibroblast growth factor 2 (FGF2), fibroblast growth factor 9 (FGF9), fibroblast growth factor 10 (FGF10), fibroblast growth factor 18 (FGF18), fibroblast growth factor 19 (FGF19), fibroblast growth factor 21 (FGF21), and fibroblast growth factor 23 (FGF23).

52. The method of 50 or 51, wherein the binding is characterized by an equilibrium dissociation constant (K_{d}) of about 0.2 nM to about 20 nM.

53. The method of 52, wherein the binding is characterized by a K_{d} of about 1 nM to about 10 nM, wherein optionally the K_{d} is about 1 nm, about 2 nm, about 3 nm, about 4 nm, about 5 nm, about 6 nm, about 7 nm, about 8 nm, about 9 nm, or about 10 nm.

54. The method of any one of 1 to 53, wherein the amino acid sequence of the sFGFR3 polypeptide is set forth in SEQ ID NO: 5.

55. The method of any one of 1 to 54, wherein the sFGFR3 polypeptide comprises a signal peptide, such as a signal peptide of a naturally-occurring FGFR3 polypeptide.

56. The method of 55, wherein the signal peptide comprises the amino acid sequence of SEQ ID NO: 21.

57. The method of any one of 1 to 56, wherein the sFGFR3 polypeptide comprises a heterologous polypeptide.

58. The method of 57, wherein the heterologous polypeptide is a fragment crystallizable region of an immunoglobulin (Fc region) or human serum albumin (HSA).

59. The method of any one of 1 to 59, wherein the polynucleotide encoding the sFGFR3 polypeptide comprises a nucleic acid sequence having at least 85% and up to 100% sequence identity to the nucleic acid sequence of any one of SEQ ID NOs: 10-18.

60. The polypeptide of 59, wherein the polynucleotide consists of the nucleic acid sequence of any one of SEQ ID NOs: 10-18.

61. The method of 59 or 60, wherein the polynucleotide is an isolated polynucleotide.

62. The method of 59 or 60, wherein the polynucleotide is in a vector.

63. The method of 62, wherein the vector is selected from the group consisting of a plasmid, an artificial chromosome, a viral vector, and a phage vector.

64. The method of 62 or 63, wherein vector is in the host cell.

65. The method of 64, wherein the host cell is an isolated host cell.

66. The method of 65, wherein the host cell is from the subject.

67. The method of 66, wherein the host cell has been transformed with the polynucleotide.

68. The method of 64 or 65, wherein the host cell is a HEK 293 cell or CHO cell.

69. The method of any one of 1 to 68, wherein the sFGFR3 is administered as a composition comprising a pharmaceutically acceptable excipient, carrier, or diluent.

70. The method of 69, wherein the composition is administered to the subject at a dose of about 0.001 mg/kg to about 30 mg/kg of the sFGFR3 polypeptide.

71. The method of 70, wherein the composition is administered at a dose of about 0.01 mg/kg to about 10 mg/kg of the sFGFR3 polypeptide.

72. The method of any one of 69 to 71, wherein the composition is administered daily, weekly, or monthly.

73. The method of any one of 69 to 72, wherein the composition is administered seven times a week, six times a week, five times a week, four times a week, three times a week, twice a week, weekly, every two weeks, or once a month.

74. The method of 73, wherein the composition is administered at a dose of about 2.5 mg/kg to about 10 mg/kg of the sFGFR3 polypeptide once or twice a week.

75. The method of any one of 69 to 74, wherein the composition is administered by parenteral administration, enteral administration, or topical administration.

76. The method of 75, wherein the composition is administered by subcutaneous administration, intravenous administration, intramuscular administration, intra-arterial administration, intrathecal administration, or intraperitoneal administration.

77. The method of 76, wherein the composition is administered by subcutaneous administration.

78. The method of any one of 1 to 77, wherein the subject has not been previously administered the sFGFR3 polypeptide.

79. The method of any one of 1 to 78, wherein the subject is a human.

80. The method of any one of 1 to 79, wherein the sFGFR3 polypeptide has an in vivo half-life of between about 2 hours to about 25 hours.

81. A composition comprising a soluble fibroblast growth factor receptor 3 (sFGFR3) polypeptide, a polynucleotide encoding the sFGFR3 polypeptide, or a host cell comprising the polynucleotide for treating or reducing abnormal fat distribution in a subject in need thereof, such as by the method of any one of 1 to 80.

82. Use of a soluble fibroblast growth factor receptor 3 (sFGFR3) polypeptide, a polynucleotide encoding the sFGFR3 polypeptide, or a host cell comprising a polynucleotide encoding the sFGFR3 polypeptide in the manufacture of a medicament for treating or reducing abnormal fat distribution in a subject in need thereof, such as by the method of any one of 1 to 80.

1. A method of treating or reducing abnormal fat deposition in a subject in need thereof comprising administering a soluble fibroblast growth factor receptor 3 (sFGFR3) polypeptide, a polynucleotide encoding the sFGFR3 polypeptide, or a host cell comprising the polynucleotide to the subject.

2. The method of claim 1, wherein the abnormal fat deposition comprises visceral fat deposition.

3. The method of claim 2, wherein:
a) the abnormal visceral fat deposition is associated with or surrounding one or more of the following organs: the heart, liver, spleen, kidneys, pancreas, intestines, reproductive organs, and gall bladder;
b) the abnormal visceral fat deposition causes disease in one or more of the following organs: the heart, lungs, trachea, liver, pancreas, brain, reproductive organs, arteries, and gall bladder; or
c) the abnormal visceral fat deposition is caused by dysfunction in an endocrine organ, such as an adrenal gland, a pituitary gland, or a reproductive organ, such as an ovary.

4. The method of any one of claims 1 to 3, wherein the method reduces or eliminates one or more conditions associated with the abnormal fat distribution.

5. The method of claim 4, wherein the one or more conditions are selected from the group consisting of obstructive sleep apnea, pulmonary disease, cardiovascular disease, metabolic disease, neurological disease, dyslipidemia, hypertension, atherosclerosis, myocardial infarction, stroke, dementia, infertility, menstrual irregularities, insulin dysregulation, and glucose dysregulation.

6. The method of claim 5, wherein the dyslipidemia comprises an abnormal level of one or more of triglycerides, high-density lipoproteins (HDLs), low-density lipoproteins (LDLs), and cholesterol.

7. The method of claim 5, wherein the cardiovascular disease is heart disease or stroke.

8. The method of claim 5, wherein the pulmonary disease is asthma and restrictive lung disease.

9. The method of claim 5, wherein the neurological disease is dementia or Alzheimer's disease.

10. The method of claim 5, wherein the metabolic disease is type 2 diabetes, glucose intolerance, nonalcoholic fatty liver disease and liver toxicity.

11. The method of claim 5, wherein the insulin dysregulation is insulin resistance.

12. The method of any one of claims 1-11, wherein the subject is not overweight or lacks substantial subcutaneous fat deposition.

13. The method of any one of claims 1-12, wherein the abnormal fat deposition is determined using an anthropometric technique, or imaging.

14. The method of claim 13, wherein the anthropometric technique is body mass index (BMI) or android:gynoid fat ratio.

15. The method of claim 13, wherein the imaging comprises computed tomography (CT), magnetic resonance imaging (MRI), and dual energy x-ray absorptioometry (DXA).

16. The method of any one of claims 1-15, wherein the subject does not exhibit substantial abnormal fat deposition outside the abdomen.

17. The method of any one of claims 1-16, wherein the subject is a fetus, a neonate, an infant, a child, a juvenile, an adolescent, or an adult.

18. The method of any one of claims 1-17, wherein the method reduces visceral fat deposition.

19. The method of any one of claims 1-18, wherein the sFGFR3 polypeptide comprises at least 50 consecutive amino acids of an extracellular domain of a naturally occurring fibroblast growth factor receptor 3 (FGFR3) polypeptide.

20. The method of claim 19, wherein the sFGFR3 polypeptide comprises 100-370 consecutive amino acids of an extracellular domain of the naturally occurring FGFR3 polypeptide.

21. The method of claim 19 or 20, wherein the sFGFR3 polypeptide comprises fewer than 350 amino acids of the extracellular domain of the naturally occurring FGFR3 polypeptide.

22. The method of any one of claims 19-21, wherein the sFGFR3 polypeptide comprises an Ig-like C2-type domain 1, 2, and/or 3 of the naturally occurring FGFR3 polypeptide.

23. The method of any one of claims 1-22, wherein the sFGFR3 polypeptide lacks a signal peptide and/or a transmembrane domain, such as the signal peptide and/or transmembrane domain of a naturally occurring FGFR3 polypeptide.

24. The method of any one of claims 1-23, wherein the sFGFR3 polypeptide is a mature polypeptide.

25. The method of any one of claims 1-24, wherein the sFGFR3 polypeptide comprises 400 consecutive amino acids or fewer of an intracellular domain of a naturally-occurring FGFR3 polypeptide.

26. The method of claim 25, wherein the sFGFR3 polypeptide comprises between 5 and 399 consecutive amino acids of the intracellular domain of a naturally-occurring FGFR3 polypeptide, such as 175, 150, 125, 100, 75, 50, 40, 30, 20, 15, or fewer consecutive amino acids of the intracellular domain of a naturally-occurring FGFR3 polypeptide.

27. The method of claim 26, wherein the sFGFR3 polypeptide comprises an amino acid sequence having at least 90%, 92%, 95%, 97%, or 99% sequence identity to amino acids 401 to 413 of SEQ ID NO: 8.

28. The method of claim 27, wherein the sFGFR3 polypeptide comprises amino acids 401 to 413 of SEQ ID NO: 8.

29. The method of any one of claims 1-28, wherein the sFGFR3 polypeptide lacks a tyrosine kinase domain of a naturally-occurring FGFR3 polypeptide.

30. The method of any one of claims 1-29, wherein the sFGFR3 polypeptide lacks an intracellular domain of a naturally-occurring FGFR3 polypeptide.

31. The method of any one of claims 1-30, wherein the sFGFR3 polypeptide comprises fewer than 475, 450, 425, 400, 375, 350, 300, 250, 200, 150, or 100 amino acids in length.

32. The method of any one of claims 1-31, wherein the sFGFR3 polypeptide comprises an amino acid sequence having at least 85% sequence identity to amino acids residues 1 to 280 of SEQ ID NO: 8.

33. The method of claim 32, wherein the amino acid sequence of the sFGFR3 polypeptide has 86%-100% sequence identity to amino acids residues 1 to 280 of SEQ ID NO: 8.

34. The method of any one of claims 1-33, wherein the sFGFR3 polypeptide comprises an amino acid sequence having at least 85% sequence identity to the sequence of any one of SEQ ID NOs: 1-7.

35. The method of claim 34, wherein the amino acid sequence of the sFGFR3 polypeptide has 86%-100% sequence identity to the sequence of any one of SEQ ID NOs: 1-7.

36. The method of any one of claims 1-35, wherein the subject has a skeletal growth retardation disorder, obesity, polycystic ovary syndrome, or hypercortisolism, such as Cushing's disease.

37. The method of claim 36, wherein the skeletal growth retardation disorder is a FGFR3-related skeletal disease.

38. The method of claim 37, wherein the FGFR3-related skeletal disease is selected from the group consisting of achondroplasia, thanatophoric dysplasia type I (TDI), thanatophoric dysplasia type II (TDII), severe achondroplasia with developmental delay and Acanthosis nigricans (SADDEN), hypochondroplasia, a craniosynostosis syndrome, and camptodactyly, tall stature, and hearing loss syndrome (CATSHL).

39. The method of claim 38, wherein the FGFR3-related skeletal disease is achondroplasia.

40. The method of claim 38, wherein the craniosynostosis syndrome is selected from the group consisting of Muenke syndrome, Crouzon syndrome, and Crouzonoderrnoskeletal syndrome.

41. The method of any one of claims 37-40, wherein the FGFR3-related skeletal disease is caused by expression in the subject of a FGFR3 variant that exhibits ligand-dependent overactivation.

42. The method of claim 41, wherein the FGFR3 variant comprises an amino acid substitution of a glycine residue with an arginine residue at position 358 (G358R) as set forth in SEQ ID NO: 9.

43. The method of any one of claims 36-42, wherein the subject has been diagnosed with the skeletal growth retardation disorder, obesity, polycystic ovary syndrome, or hypercortisolism, such as Cushing's disease.

44. The method of any one of claims 36-43, wherein the subject exhibits one or more symptoms of the skeletal growth retardation disorder selected from the group consisting of short limbs, short trunk, bowlegs, a waddling gait, skull malformations, cloverleaf skull, craniosynostosis, wormian bones, anomalies of the hands, anomalies of the feet, hitchhiker thumb, and chest anomalies.

45. The method of any one of claims 1-35, wherein the subject does not have a skeletal growth retardation disorder, such as a FGFR3-related skeletal disease.

46. The method of claim 45, wherein the subject does not have an FGFR3-related skeletal disease is selected from the group consisting of achondroplasia, thanatophoric dysplasia type I (TDI), thanatophoric dysplasia type II (TDII), severe achondroplasia with developmental delay and Acanthosis nigricans (SADDEN), hypochondroplasia, a craniosynostosis syndrome, and camptodactyly, tall stature, and hearing loss syndrome (CATSHL).

47. The method of any one of claims 19-23 and 25-30, wherein the naturally-occurring human FGFR3 polypeptide comprises the amino acid sequence of Genbank Accession No. NP_000133.

48. The method of any one of claims 1-47, wherein the sFGFR3 polypeptide binds to a fibroblast growth factor (FGF).

49. The method of claim 48, wherein the FGF is selected from the group consisting of fibroblast growth factor 1 (FGF1), fibroblast growth factor 2 (FGF2), fibroblast growth factor 9 (FGF9), fibroblast growth factor 10 (FGF10), fibroblast growth factor 18 (FGF18), fibroblast growth factor 19 (FGF19), fibroblast growth factor 21 (FGF21), and fibroblast growth factor 23 (FGF23).

50. The method of claim 48 or 49, wherein the binding is characterized by an equilibrium dissociation constant (K_{d}) of about 0.2 nM to about 20 nM.

51. The method of claim 50, wherein the binding is characterized by a K_{d} of about 1 nM to about 10 nM, wherein optionally the K_{d} is about 1 nm, about 2 nm, about 3 nm, about 4 nm, about 5 nm, about 6 nm, about 7 nm, about 8 nm, about 9 nm, or about 10 nm.

52. The method of any one of claims 1-51, wherein the amino acid sequence of the sFGFR3 polypeptide is set forth in SEQ ID NO: 5.

53. The method of any one of claims 1-52, wherein the sFGFR3 polypeptide comprises a signal peptide, such as a signal peptide of a naturally-occurring FGFR3 polypeptide.

54. The method of claim 53, wherein the signal peptide comprises the amino acid sequence of SEQ ID NO: 21.

55. The method of any one of claims 1-54, wherein the sFGFR3 polypeptide comprises a heterologous polypeptide.

56. The method of claim 55, wherein the heterologous polypeptide is a fragment crystallizable region of an immunoglobulin (Fc region) or human serum albumin (HSA).

57. The method of any one of claims 1-56, wherein the polynucleotide encoding the sFGFR3 polypeptide comprises a nucleic acid sequence having at least 85% and up to 100% sequence identity to the nucleic acid sequence of any one of SEQ ID NOs: 10-18.

58. The polypeptide of claim 57, wherein the polynucleotide consists of the nucleic acid sequence of any one of SEQ ID NOs: 10-18.

59. The method of claim 57 or 58, wherein the polynucleotide is an isolated polynucleotide.

60. The method of claim 57 or 58, wherein the polynucleotide is in a vector.

61. The method of claim 60, wherein the vector is selected from the group consisting of a plasmid, an artificial chromosome, a viral vector, and a phage vector.

62. The method of claim 60 or 61, wherein vector is in the host cell.

63. The method of claim 62, wherein the host cell is an isolated host cell.

64. The method of claim 63, wherein the host cell is from the subject.

65. The method of claim 64, wherein the host cell has been transformed with the polynucleotide.

66. The method of claim 62 or 63, wherein the host cell is a HEK 293 cell or CHO cell.

67. The method of any one of claims 1-66, wherein the sFGFR3 is administered as a composition comprising a pharmaceutically acceptable excipient, carrier, or diluent.

68. The method of claim 67, wherein the composition is administered to the subject at a dose of about 0.001 mg/kg to about 30 mg/kg of the sFGFR3 polypeptide.

69. The method of claim 68, wherein the composition is administered at a dose of about 0.01 mg/kg to about 10 mg/kg of the sFGFR3 polypeptide.

70. The method of any one of claims 67-69, wherein the composition is administered daily, weekly, or monthly.

71. The method of any one of claims 67-70, wherein the composition is administered seven times a week, six times a week, five times a week, four times a week, three times a week, twice a week, weekly, every two weeks, or once a month.

72. The method of claim 71, wherein the composition is administered at a dose of about 2.5 mg/kg to about 10 mg/kg of the sFGFR3 polypeptide once or twice a week.

73. The method of any one of claims 67-72, wherein the composition is administered by parenteral administration, enteral administration, or topical administration.

74. The method of claim 73, wherein the composition is administered by subcutaneous administration, intravenous administration, intramuscular administration, intra-arterial administration, intrathecal administration, or intraperitoneal administration.

75. The method of claim 74, wherein the composition is administered by subcutaneous administration.

76. The method of any one of claims 1-75, wherein the subject has not been previously administered the sFGFR3 polypeptide.

77. The method of any one of claims 1-76, wherein the subject is a human.

78. The method of any one of claims 1-77, wherein the sFGFR3 polypeptide has an in vivo half-life of between about 2 hours to about 25 hours.

79. A composition comprising a soluble fibroblast growth factor receptor 3 (sFGFR3) polypeptide, a polynucleotide encoding the sFGFR3 polypeptide, or a host cell comprising the polynucleotide for treating or reducing abnormal fat distribution in a subject in need thereof.

80. The composition of claim 79, wherein the abnormal fat deposition comprises visceral fat deposition.

81. The composition of claim 80, wherein:
a) the visceral fat deposition is associated with or surrounding one or more of the following organs: the heart, liver, spleen, kidneys, pancreas, intestines, reproductive organs, and gall bladder;
b) the visceral fat deposition causes disease in one or more of the following organs: the heart, lungs, trachea, liver, pancreas, brain, reproductive organs, arteries, and gall bladder; or
c) the visceral fat deposition is caused by dysfunction in an endocrine organ, such as an adrenal gland, a pituitary gland, or a reproductive organ, such as an ovary.

82. The composition of any one of claims 79-81, wherein the composition reduces or eliminates one or more conditions associated with the abnormal fat distribution.

83. The composition of claim 82, wherein the one or more conditions are selected from the group consisting of obstructive sleep apnea, pulmonary disease, cardiovascular disease, metabolic disease, neurological disease, dyslipidemia, hypertension, atherosclerosis, myocardial infarction, stroke, dementia, infertility, menstrual irregularities, insulin dysregulation, and glucose dysregulation.

84. The composition of claim 83, wherein the dyslipidemia comprises an abnormal level of one or more of triglycerides, high-density lipoproteins (HDLs), low-density lipoproteins (LDLs), and cholesterol.

85. The composition of claim 83, wherein the cardiovascular disease is heart disease or stroke.

86. The composition of claim 83, wherein the pulmonary disease is asthma and restrictive lung disease.

87. The composition of claim 83, wherein the neurological disease is dementia or Alzheimer's disease.

88. The composition of claim 83, wherein the metabolic disease is type 2 diabetes, glucose intolerance, nonalcoholic fatty liver disease and liver toxicity.

89. The composition of claim 83, wherein the insulin dysregulation is insulin resistance.

90. The composition of any one of claims 79-89, wherein the subject is not overweight or lacks substantial subcutaneous fat deposition.

91. The composition of any one of claims 79-90, wherein the abnormal fat deposition is determined using an anthropometric technique, or imaging.

92. The composition of claim 91, wherein the anthropometric technique is body mass index (BMI) or android:gynoid fat ratio.

93. The composition of claim 91, wherein the imaging comprises computed tomography (CT), magnetic resonance imaging (MRI), and dual energy x-ray absorptioometry (DXA).

94. The composition of any one of claims 79-93, wherein the subject does not exhibit substantial abnormal fat deposition outside the abdomen.

95. The composition of any one of claims 79-94, wherein the subject is a fetus, a neonate, an infant, a child, a juvenile, an adolescent, or an adult.

96. The composition of any one of claims 79-95, wherein the composition reduces visceral fat deposition.

97. The composition of any one of claims 79-96, wherein the sFGFR3 polypeptide comprises at least 50 consecutive amino acids of an extracellular domain of a naturally occurring fibroblast growth factor receptor 3 (FGFR3) polypeptide.

98. The composition of claim 97, wherein the sFGFR3 polypeptide comprises 100-370 consecutive amino acids of an extracellular domain of the naturally occurring FGFR3 polypeptide.

99. The composition of claim 97 or 98, wherein the sFGFR3 polypeptide comprises fewer than 350 amino acids of the extracellular domain of the naturally occurring FGFR3 polypeptide.

100. The composition of any one of claims 97-99, wherein the sFGFR3 polypeptide comprises an Ig-like 02-type domain 1, 2, and/or 3 of the naturally occurring FGFR3 polypeptide.

101. The composition of any one of claims 79-100, wherein the sFGFR3 polypeptide lacks a signal peptide and/or a transmembrane domain, such as the signal peptide and/or transmembrane domain of a naturally occurring FGFR3 polypeptide.

102. The composition of any one of claims 79-101, wherein the sFGFR3 polypeptide is a mature polypeptide.

103. The composition of any one of claims 79-102, wherein the sFGFR3 polypeptide comprises 400 consecutive amino acids or fewer of an intracellular domain of a naturally-occurring FGFR3 polypeptide.

104. The composition of claim 103, wherein the sFGFR3 polypeptide comprises between 5 and 399 consecutive amino acids of the intracellular domain of a naturally-occurring FGFR3 polypeptide, such as 175, 150, 125, 100, 75, 50, 40, 30, 20, 15, or fewer consecutive amino acids of the intracellular domain of a naturally-occurring FGFR3 polypeptide.

105. The composition of claim 104, wherein the sFGFR3 polypeptide comprises an amino acid sequence having at least 90%, 92%, 95%, 97%, or 99% sequence identity to amino acids 401 to 413 of SEQ ID NO: 8.

106. The composition of claim 105, wherein the sFGFR3 polypeptide comprises amino acids 401 to 413 of SEQ ID NO: 8.

107. The composition of any one of claims 79-106, wherein the sFGFR3 polypeptide lacks a tyrosine kinase domain of a naturally-occurring FGFR3 polypeptide.

108. The composition of any one of claims 79-107, wherein the sFGFR3 polypeptide lacks an intracellular domain of a naturally-occurring FGFR3 polypeptide.

109. The composition of any one of claims 79-108, wherein the sFGFR3 polypeptide comprises fewer than 475, 450, 425, 400, 375, 350, 300, 250, 200, 150, or 100 amino acids in length.

110. The composition of any one of claims 79-109, wherein the sFGFR3 polypeptide comprises an amino acid sequence having at least 85% sequence identity to amino acids residues 1 to 280 of SEQ ID NO: 8.

111. The composition of claim 110, wherein the amino acid sequence of the sFGFR3 polypeptide has 86%-100% sequence identity to amino acids residues 1 to 280 of SEQ ID NO: 8.

112. The composition of any one of claims 79-111, wherein the sFGFR3 polypeptide comprises an amino acid sequence having at least 85% sequence identity to the sequence of any one of SEQ ID NOs: 1-7.

113. The composition of claim 112, wherein the amino acid sequence of the sFGFR3 polypeptide has 86%-100% sequence identity to the sequence of any one of SEQ ID NOs: 1-7.

114. The composition of any one of claims 79-113, wherein the subject has a skeletal growth retardation disorder, obesity, polycystic ovary syndrome, or hypercortisolism, such as Cushing's disease.

115. The composition of claim 114, wherein the skeletal growth retardation disorder is a FGFR3-related skeletal disease.

116. The composition of claim 115, wherein the FGFR3-related skeletal disease is selected from the group consisting of achondroplasia, thanatophoric dysplasia type I (TDI), thanatophoric dysplasia type II (TDI I), severe achondroplasia with developmental delay and Acanthosis nigricans (SADDEN), hypochondroplasia, a craniosynostosis syndrome, and camptodactyly, tall stature, and hearing loss syndrome (CATSHL).

117. The composition of claim 116, wherein the FGFR3-related skeletal disease is achondroplasia.

118. The composition of claim 116, wherein the craniosynostosis syndrome is selected from the group consisting of Muenke syndrome, Crouzon syndrome, and Crouzonodermoskeletal syndrome.

119. The composition of any one of claims 115-118, wherein the FGFR3-related skeletal disease is caused by expression in the subject of a FGFR3 variant that exhibits ligand-dependent overactivation.

120. The composition of claim 119, wherein the FGFR3 variant comprises an amino acid substitution of a glycine residue with an arginine residue at position 358 (G358R) as set forth in SEQ ID NO: 9.

121. The composition of any one of claims 114-120, wherein the subject has been diagnosed with the skeletal growth retardation disorder, obesity, polycystic ovary syndrome, or hypercortisolism, such as Cushing's disease.

122. The composition of any one of claims 114-121, wherein the subject exhibits one or more symptoms of the skeletal growth retardation disorder selected from the group consisting of short limbs, short trunk, bowlegs, a waddling gait, skull malformations, cloverleaf skull, craniosynostosis, wormian bones, anomalies of the hands, anomalies of the feet, hitchhiker thumb, and chest anomalies.

123. The composition of any one of claims 79-113, wherein the subject does not have a skeletal growth retardation disorder, such as a FGFR3-related skeletal disease.

124. The composition of claim 123, wherein the subject does not have an FGFR3-related skeletal disease selected from the group consisting of achondroplasia, thanatophoric dysplasia type I (TDI), thanatophoric dysplasia type II (TDII), severe achondroplasia with developmental delay and Acanthosis nigricans (SADDEN), hypochondroplasia, a craniosynostosis syndrome, and camptodactyly, tall stature, and hearing loss syndrome (CATSHL).

125. The composition of any one of claims 97-101 and 103-108, wherein the naturally-occurring human FGFR3 polypeptide comprises the amino acid sequence of Genbank Accession No. NP_000133.

126. The composition of any one of claims 79-125, wherein the sFGFR3 polypeptide binds to a fibroblast growth factor (FGF).

127. The composition of claim 126, wherein the FGF is selected from the group consisting of fibroblast growth factor 1 (FGF1), fibroblast growth factor 2 (FGF2), fibroblast growth factor 9 (FGF9), fibroblast growth factor 10 (FGF10), fibroblast growth factor 18 (FGF18), fibroblast growth factor 19 (FGF19), fibroblast growth factor 21 (FGF21), and fibroblast growth factor 23 (FGF23).

128. The composition of claim 126 or 127, wherein the binding is characterized by an equilibrium dissociation constant (K_{d}) of about 0.2 nM to about 20 nM.

129. The composition of claim 128, wherein the binding is characterized by a K_{d} of about 1 nM to about 10 nM, wherein optionally the K_{d} is about 1 nm, about 2 nm, about 3 nm, about 4 nm, about 5 nm, about 6 nm, about 7 nm, about 8 nm, about 9 nm, or about 10 nm.

130. The composition of any one of claims 79-129, wherein the amino acid sequence of the sFGFR3 polypeptide is set forth in SEQ ID NO: 5.

131. The composition of any one of claims 79-130, wherein the sFGFR3 polypeptide comprises a signal peptide, such as a signal peptide of a naturally-occurring FGFR3 polypeptide.

132. The composition of claim 131, wherein the signal peptide comprises the amino acid sequence of SEQ ID NO: 21.

133. The composition of any one of claims 79-132, wherein the sFGFR3 polypeptide comprises a heterologous polypeptide.

134. The composition of claim 133, wherein the heterologous polypeptide is a fragment crystallizable region of an immunoglobulin (Fc region) or human serum albumin (HSA).

135. The composition of any one of claims 79-134, wherein the polynucleotide encoding the sFGFR3 polypeptide comprises a nucleic acid sequence having at least 85% and up to 100% sequence identity to the nucleic acid sequence of any one of SEQ ID NOs: 10-18.

136. The composition of claim 135, wherein the polynucleotide consists of the nucleic acid sequence of any one of SEQ ID NOs: 10-18.

137. The composition of claim 135 or 136, wherein the polynucleotide is an isolated polynucleotide.

138. The composition of claim 135 or 136, wherein the polynucleotide is in a vector.

139. The composition of claim 138, wherein the vector is selected from the group consisting of a plasmid, an artificial chromosome, a viral vector, and a phage vector.

140. The composition of claim 138 or 139, wherein vector is in the host cell.

141. The composition of claim 140, wherein the host cell is an isolated host cell.

142. The composition of claim 141, wherein the host cell is from the subject.

143. The composition of claim 142, wherein the host cell has been transformed with the polynucleotide.

144. The composition of claim 140 or 141, wherein the host cell is a HEK 293 cell or CHO cell.

145. The composition of any one of claims 79-144, further comprising a pharmaceutically acceptable excipient, carrier, or diluent.

146. The composition of claim 145, wherein the composition is formulated for administration to the subject at a dose of about 0.001 mg/kg to about 30 mg/kg of the sFGFR3 polypeptide.

147. The composition of claim 146, wherein the composition is formulated for administration to the subject at a dose of about 0.01 mg/kg to about 10 mg/kg of the sFGFR3 polypeptide.

148. The composition of any one of claims 145-147, wherein the composition is formulated for administration to the subject daily, weekly, or monthly.

149. The composition of any one of claims 145-148, wherein the composition is formulated for administration to the subject seven times a week, six times a week, five times a week, four times a week, three times a week, twice a week, weekly, every two weeks, or once a month.

150. The composition of claim 149, wherein the composition is formulated for administration to the subject at a dose of about 2.5 mg/kg to about 10 mg/kg of the sFGFR3 polypeptide once or twice a week.

151. The composition of any one of claims 145-150, wherein the composition is formulated for administration to the subject by parenteral administration, enteral administration, or topical administration.

152. The composition of claim 151, wherein the composition is formulated for administration to the subject by subcutaneous administration, intravenous administration, intramuscular administration, intra-arterial administration, intrathecal administration, or intraperitoneal administration.

153. The composition of claim 152, wherein the composition is formulated for administration to the subject by subcutaneous administration.

154. The composition of any one of claims 79-153, wherein the subject has not been previously administered the sFGFR3 polypeptide.

155. The composition of any one of claims 79-154, wherein the subject is a human.

156. The composition of any one of claims 79-155, wherein the sFGFR3 polypeptide has an in vivo half-life of between about 2 hours to about 25 hours.

157. Use of a soluble fibroblast growth factor receptor 3 (sFGFR3) polypeptide, a polynucleotide encoding the sFGFR3 polypeptide, or a host cell comprising a polynucleotide encoding the sFGFR3 polypeptide in the manufacture of a medicament for treating or reducing abnormal fat distribution in a subject in need thereof, such as by the method of any one of claims 1-78.
